SEQUENCE LISTING (5 sequences)

SEQ ID NO: 1 - protein, 1379 aa, Artificial sequence
  Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 2 - DNA, 13890 bp, Artificial sequence
  Synthetic polynucleotide
gtgccgaattcggatccggagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactcgacttttacaacaattaccaacaacaacaaacaacaaacaacattacaattactatttacaattatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagccgctgcaagaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaattcgagctcggagtgatcaaaagtcccacatcgatcaggtgatatatagcagcttagtttatataatgatagagtcgacatagcgattgatatacaacaatggctgcagttttagagctagaaatagcaagttaaaataaggctagtccgttatcaacttgaaaaagtggcaccgagtcggtgctttttttctagacccagctttcttgtacaaagttggcattacgctttacgaattcccatggggagtgatcaaaagtcccacatcgatcaggtgatatatagcagcttagtttatataatgatagagtcgacatagcgattgaccttatcccctgcctttagttttagagctagaaatagcaagttaaaataaggctagtccgttatcaacttgaaaaagtggcaccgagtcggtgctttttttctagacccagctttcttgtacaaagttggcattacgctcagagaattcgcatgcggagtgatcaaaagtcccacatcgatcaggtgatatatagcagcttagtttatataatgatagagtcgacatagcgattgaaacaccaaattatgttgtgttttagagctagaaatagcaagttaaaataaggctagtccgttatcaacttgaaaaagtggcaccgagtcggtgctttttttctagacccagctttcttgtacaaagttggcattacgcttgtggaattcctcgagggagtgatcaaaagtcccacatcgatcaggtgatatatagcagcttagtttatataatgatagagtcgacatagcgattgagatccatagtacagtactgttttagagctagaaatagcaagttaaaataaggctagtccgttatcaacttgaaaaagtggcaccgagtcggtgctttttttctagacccagctttcttgtacaaagttggcattacgctgagcgaattccatatgggagtgatcaaaagtcccacatcgatcaggtgatatatagcagcttagtttatataatgatagagtcgacatagcgattgcagtaacaagacagagtgagttttagagctagaaatagcaagttaaaataaggctagtccgttatcaacttgaaaaagtggcaccgagtcggtgctttttttctagacccagctttcttgtacaaagttggcattacgcttgccgaattcggatccggagtgatcaaaagtcccacatcgatcaggtgatatatagcagcttagtttatataatgatagagtcgacatagcgattggtccaacaatatatgtttatgttttagagctagaaatagcaagttaaaataaggctagtccgttatcaacttgaaaaagtggcaccgagtcggtgctttttttctagacccagctttcttgtacaaagttggcattacgctgcaagaattcaagcttggagtgatcaaaagtcccacatcgatcaggtgatatatagcagcttagtttatataatgatagagtcgacatagcgattgacaccactcgatttaaattgttttagagctagaaatagcaagttaaaataaggctagtccgttatcaacttgaaaaagtggcaccgagtcggtgctttttttctagacccagctttcttgtacaaagttggcattacgctactagaattcgagctcggagtgatcaaaagtcccacatcgatcaggtgatatatagcagcttagtttatataatgatagagtcgacatagcgattgcaatgcaagtagctgcaaagttttagagctagaaatagcaagttaaaataaggctagtccgttatcaacttgaaaaagtggcaccgagtcggtgctttttttctagacccagctttcttgtacaaagttggcattacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 3 - DNA, 13890 bp, Artificial sequence
  Synthetic polynucleotide
gtgccgaattcggatccggagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactcgacttttacaacaattaccaacaacaacaaacaacaaacaacattacaattactatttacaattatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagccgctgcaagaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaattcgagctcggagtgatcaaaagtcccacatcgatcaggtgatatatagcagcttagtttatataatgatagagtcgacatagcgattaaagagttgtagttgtttttgttttagagctagaaatagcaagttaaaataaggctagtccgttatcaacttgaaaaagtggcaccgagtcggtgctttttttctagacccagctttcttgtacaaagttggcattacgctttacgaattcccatggggagtgatcaaaagtcccacatcgatcaggtgatatatagcagcttagtttatataatgatagagtcgacatagcgattagatcattagagagtcagatgttttagagctagaaatagcaagttaaaataaggctagtccgttatcaacttgaaaaagtggcaccgagtcggtgctttttttctagacccagctttcttgtacaaagttggcattacgctcagagaattcgcatgcggagtgatcaaaagtcccacatcgatcaggtgatatatagcagcttagtttatataatgatagagtcgacatagcgattgaaaggtgagagcttgttgtgttttagagctagaaatagcaagttaaaataaggctagtccgttatcaacttgaaaaagtggcaccgagtcggtgctttttttctagacccagctttcttgtacaaagttggcattacgcttgtggaattcctcgagggagtgatcaaaagtcccacatcgatcaggtgatatatagcagcttagtttatataatgatagagtcgacatagcgattaaaatagctcaaatcggagggttttagagctagaaatagcaagttaaaataaggctagtccgttatcaacttgaaaaagtggcaccgagtcggtgctttttttctagacccagctttcttgtacaaagttggcattacgctgagcgaattccatatgggagtgatcaaaagtcccacatcgatcaggtgatatatagcagcttagtttatataatgatagagtcgacatagcgattgaatgtggagctaaatgtaagttttagagctagaaatagcaagttaaaataaggctagtccgttatcaacttgaaaaagtggcaccgagtcggtgctttttttctagacccagctttcttgtacaaagttggcattacgcttgccgaattcggatccggagtgatcaaaagtcccacatcgatcaggtgatatatagcagcttagtttatataatgatagagtcgacatagcgattggtgtaggtactacctaaaaggttttagagctagaaatagcaagttaaaataaggctagtccgttatcaacttgaaaaagtggcaccgagtcggtgctttttttctagacccagctttcttgtacaaagttggcattacgctgcaagaattcaagcttggagtgatcaaaagtcccacatcgatcaggtgatatatagcagcttagtttatataatgatagagtcgacatagcgattgtagagattgtttgtaataagttttagagctagaaatagcaagttaaaataaggctagtccgttatcaacttgaaaaagtggcaccgagtcggtgctttttttctagacccagctttcttgtacaaagttggcattacgctactagaattcgagctcggagtgatcaaaagtcccacatcgatcaggtgatatatagcagcttagtttatataatgatagagtcgacatagcgattggtggtagtaattgtgagtagttttagagctagaaatagcaagttaaaataaggctagtccgttatcaacttgaaaaagtggcaccgagtcggtgctttttttctagacccagctttcttgtacaaagttggcattacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 4 - DNA, 2744 bp, Artificial sequence
  Synthetic polynucleotide
cacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttcacgacacggaacaattaaggttttagagctagaaatagcaagttaaaataaggctagtccgttatcaacttgaaaaagtggcaccgagtcggtgcttttttttacgtacaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccccgcccgtttggacatatatgttttagagctagaaatagcaagttaaaataaggctagtccgttatcaacttgaaaaagtggcaccgagtcggtgctttttttgttaactaagaacgaactaagccggacaaaaaaaggagcacatatacaaaccggttttattcatgaatggtcacgatggatgatggggctcagacttgagctacgaggccgcaggcgagagaagcctagtgtgctctctgcttgtttgggccgtaacggaggatacggccgacgagcgtgtactaccgcgcgggatgccgctgggcgctgcgggggccgttggatggggatcggtgggtcgcgggagcgttgaggggagacaggtttagtaccacctcgcctaccgaacaatgaagaacccaccttataaccccgcgcgctgccgcttgtgttgaacaaagcaccagtggtctagtggtagaatagtaccctgccacggtacagacccgggttcgattcccggctggtgcaggtagatcgcgtgcgtacagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagacacggacacagtggcaccgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagatacccgtatagacaagttgttttagagctagaaatagcaagttaaaataaggctagtccgttatcaacttgaaaaagtggcaccgagtcggtgctttttttttggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgcttgtactttactccgtaggttttagagctagaaatagcaagttaaaataaggctagtccgttatcaacttgaaaaagtggcaccgagtcggtgctttttttgttaaccacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgctttccaaactgatgcgtacgttttagagctagaaatagcaagttaaaataaggctagtccgttatcaacttgaaaaagtggcaccgagtcggtgcttttttttacgtacaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccggggccgcggcggtacttatgttttagagctagaaatagcaagttaaaataaggctagtccgttatcaacttgaaaaagtggcaccgagtcggtgctttttttgttaacggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgttatacacaccgcggttttgttttagagctagaaatagcaagttaaaataaggctagtccgttatcaacttgaaaaagtggcaccgagtcggtgctttttttgttaac

SEQ ID NO: 5 - DNA, 2744 bp, Artificial sequence
  Synthetic polynucleotide
cacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttggtcaagagcaaccaaacaagttttagagctagaaatagcaagttaaaataaggctagtccgttatcaacttgaaaaagtggcaccgagtcggtgcttttttttacgtacaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccgcaccagtagagattggctcgttttagagctagaaatagcaagttaaaataaggctagtccgttatcaacttgaaaaagtggcaccgagtcggtgctttttttgttaactaagaacgaactaagccggacaaaaaaaggagcacatatacaaaccggttttattcatgaatggtcacgatggatgatggggctcagacttgagctacgaggccgcaggcgagagaagcctagtgtgctctctgcttgtttgggccgtaacggaggatacggccgacgagcgtgtactaccgcgcgggatgccgctgggcgctgcgggggccgttggatggggatcggtgggtcgcgggagcgttgaggggagacaggtttagtaccacctcgcctaccgaacaatgaagaacccaccttataaccccgcgcgctgccgcttgtgttgaacaaagcaccagtggtctagtggtagaatagtaccctgccacggtacagacccgggttcgattcccggctggtgcaggctcgaccatgttcagactgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagcacttccactttggttttggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagcgaaaaggaatccatgctggttttagagctagaaatagcaagttaaaataaggctagtccgttatcaacttgaaaaagtggcaccgagtcggtgctttttttttggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgatcgcgggtcccacgcatagttttagagctagaaatagcaagttaaaataaggctagtccgttatcaacttgaaaaagtggcaccgagtcggtgctttttttgttaaccacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgtggtacggtcacgtgccgcgttttagagctagaaatagcaagttaaaataaggctagtccgttatcaacttgaaaaagtggcaccgagtcggtgcttttttttacgtacaaaaacatcctcacaggaaagacacgaagaaacatggtcaatggcccattatataaagcaccgccacaaagcccaaataccagttcgtcggtggagcaagtaacgcgctaggcaacaggcaaacagtttgtcccacctcgtccagtcacaaaggcaaagcgtgacttataagccagagcggaagaaccataccgagagttggtttcgcccgtcgttttagagctagaaatagcaagttaaaataaggctagtccgttatcaacttgaaaaagtggcaccgagtcggtgctttttttgttaacggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtggttttggagcaggcaagccgttttagagctagaaatagcaagttaaaataaggctagtccgttatcaacttgaaaaagtggcaccgagtcggtgctttttttgttaac

(19) United States
(12) Patent Application Publication
(10) Pub. No.: US 2020/0199604 A1
(43) Pub. Date: Jun. 25, 2020

(54) COMPOSITIONS AND METHODS FOR GENERATING WEAK ALLELES IN PLANTS

(71) Applicant: Cold Spring Harbor Laboratory, Cold Spring Harbor, NY (US)

(72) Inventors: Zachary Lippman, North Bellmore, NY (US); Daniel Rodriguez-Leal, Cold Spring Flarbor, NY (US); David Jackson, Cold Spring Harbor, NY (US)

(73) Assignee: Cold Spring Harbor Laboratory, Cold Spring Harbor, NY (US)

(21) Appl. No.: 16/613,652

(22) PCT Filed: May 17, 2018
(86) PCT No.: PCT/US2018/033143

§ 371 Date: Nov. 14, 2019

Related U.S. Application Data
(60) Provisional application No. 62/507,317, filed on May 17, 2017.

(51) Int. Cl. C12N 15/82; C12N 15/10

(52) U.S. Cl. CPC C12N 15/8213; C12N 2310/20; C12N 2800/80; C12N 15/1082

(57) ABSTRACT

Provided herein are compositions and methods for generating alleles of genes of interest in plants. In some aspects, libraries of plants or seeds are provided that comprise an expression construct comprising a RNA-guided endonuclease (e.g., a Cas9 endonuclease) and multiple different guide RNAs that target regions of the gene of interest, such as regulatory regions.

### CROSS-REFERENCE TO RELATED APPLICATIONS

This application claims the benefit of the filing date of U.S. Provisional Application No. 62/507,317, filed May 17, 2017. The entire contents of this referenced application are incorporated by reference herein.

### FEDERALLY SPONSORED RESEARCH OR DEVELOPMENT

This invention was made with government support under IOS-1546837 awarded by the National Science Foundation. The government has certain rights in the invention.

### BACKGROUND

There is an ongoing need to develop crop plants and other types of plants that have improved yield and quality, e.g., to provide more food per plant, better-tasting food, or both. There remains a need for improved methods to quickly and efficiently create and identify new alleles that improve such yield- and quality-related traits.

### SUMMARY

Provided herein is a new approach to generate useful mutations, in particular in gene regulatory regions, to generate beneficial quantitative variation in commercially relevant traits. Among other things, these traits can be incorporated into seed and plant libraries and used in plant breeding. This novel genetic approach uses RNA-guided endonuclease genome-editing to generate a variety of mutations in regulatory regions of a target gene to give rise to quantitative variations in the phenotypic effect of that gene. In particular, as described herein, a single CRISPR/RNA-guided endonuclease (e.g., CRISPR/Cas9) expression construct encoding multiple different guide RNAs can be used to generate multiple and different types of mutations within a regulatory region of a target gene. These different mutations to the target gene can produce a quantitative range of phenotypes from weak to strong. To optimize this approach, the CRISPR/RNA-guided-endonuclease-driven (e.g., CRISPR/Cas9-driven) mutagenesis is preferably performed in a heterozygous null mutant background, or alternatively, in a heterozygous hypomorphic (e.g., a moderate to strong loss-of-function) mutant background. This sensitized heterozygous mutant background allows for the identification of CRISPR/RNA-guided-endonuclease-generated (e.g., CRISPR/Cas9-generated) weak alleles that would otherwise be difficult or impossible to detect due to the subtle phenotypes generally associated with weakly penetrant mutations. The trans-generational heritability of RNA-guided endonuclease (e.g., Cas9) activity allows the CRISPR/RNA-guided endonuclease (e.g., CRISPR/Cas9) expression construct to be introduced into and then exploited in the heterozygous mutant background, allowing one to rapidly generate a wide variety of regulatory region mutants in genes that control commercially relevant traits. This approach allows for immediate selection and fixation of novel, useful alleles in transgene-free plants. For example, through rapid generation of plant and seed libraries carrying such novel alleles, this technology allows for practical expansion and enhancement of quantitative, phenotypic variation in a diverse range of traits in a wide variety of commercially relevant plants. Such alleles, and the plants and seeds carrying such alleles, enable fine-tuning of commercially relevant traits in such plants where such fine-tuning before was either impossible or impractical.

Accordingly, aspects of the disclosure relate to compositions, such as libraries of plants or seeds, and methods for generating new alleles in plants, such as alleles that weakly affect one or more plant traits, such as yield-related traits.

In some aspects, the disclosure provides a plant library or seed library. In some embodiments, the plant library comprises a plurality of F1 hybrid plants, each F1 hybrid plant in the plurality comprising: (a) a gene of interest comprising a coding sequence and having a first allele that is a hypomorphic allele or a null allele and a second allele that is different from the first allele, and (b) an expression cassette that encodes a RNA-guided endonuclease and at least four different guide RNAs (gRNAs), each gRNA containing a sequence that is complementary to a target sequence within a target region in the second allele of the gene of interest, wherein the target region is 0 to 5000 base pairs upstream of the 5′ end of the coding sequence of the gene of interest or wherein the target region is 0 to 5000 base pairs downstream of the 3′ end of the coding sequence of the gene of interest. In some embodiments, the seed library comprises a plurality of F1 hybrid seeds, each F1 hybrid seed in the plurality comprising: (a) a gene of interest comprising a coding sequence and having a first allele that is a hypomorphic allele or a null allele and a second allele that is different from the first allele, and (b) an expression cassette that encodes a RNA-guided endonuclease and at least four different guide RNAs (gRNAs), each gRNA containing a sequence that is complementary to a target sequence within a target region in the second allele of the gene of interest, wherein the target region is 0 to 5000 base pairs upstream of the 5′ end of the coding sequence of the gene of interest or wherein the target region is 0 to 5000 base pairs downstream of the 3′ end of the coding sequence of the gene of interest.

In some embodiments of the plant or seed library, the target region comprises a regulatory region of the gene of interest. In some embodiments, the regulatory region comprises a transcription factor binding site, an RNA polymerase binding site, a TATA box, or a combination of structural variations thereof. In some embodiments, the regulatory region is a promoter. In some embodiments of the plant or seed library, the expression cassette encodes at least five different gRNAs. In some embodiments, the expression cassette encodes at least six different gRNAs. In some embodiments, the expression cassette encodes at least seven different gRNAs. In some embodiments, the expression cassette encodes at least eight different gRNAs. In some embodiments, the expression cassette encodes four to nine different gRNAs. In some embodiments, the expression cassette encodes five to eight different gRNAs. In some embodiments, the expression cassette encodes six to eight different gRNAs. In some embodiments of the plant or seed library, the second allele is a naturally-occurring allele. In some embodiments, the second allele is not a hypomorphic allele. In some embodiments, the second allele is not a null allele. In some embodiments of the plant or seed library, the first allele contains a mutation in a regulatory region of the gene of interest. In some embodiments, the first allele contains a mutation in a coding sequence of the gene of interest. In some embodiments, the first allele is a hypomorphic allele that results in an mRNA expression level of the gene of interest that is at least 70% lower than an allele of the gene of interest that does not contain the mutation. In some embodiments of the plant or seed library, each target sequence is located 50 to 500 base pairs away from at least one other target sequence. In some embodiments of the plant or seed library, the library contains at least 50 members. In some embodiments, the plant or seed is a crop plant or crop seed. In some embodiments, the library is a plant library and at least one member of the library contains a gRNA/endonuclease-induced mutation in the second allele. In some embodiments, the gRNA/endonuclease-induced is a deletion, inversion, translocation or insertion, or a combination of structural variations thereof. In some embodiments of the plant or seed library, the RNA-guided endonuclease is a Cas9 or Cpf1 endonuclease.

In other aspects, the disclosure provides a method of generating a plant library or seed library. In some embodiments, the method is a method of generating a plant library comprising a plurality of F1 hybrid plants, the method comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, and (c) crossing the first plant to the second plant to produce a plurality of F1 hybrid plants, each F1 hybrid plant in the plurality comprising the first allele, the second allele and the expression cassette. In some embodiments, the method is a method of generating a seed library comprising a plurality of F1 hybrid seeds, the method comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, and (c) crossing the first plant to the second plant to produce a plurality of F1 hybrid seeds, each F1 hybrid seed in the plurality comprising the first allele, the second allele and the expression cassette.

In some embodiments of the method, the first plant is hemizygous for the expression cassette. In some embodiments of the method, the first plant is homozygous for the first allele and the second plant is homozygous for the second allele. In some embodiments of the method, the method further comprises maintaining the plurality of F1 hybrid plants or F1 hybrid seeds under conditions that permit the gRNA/endonuclease to induce mutations within the target region of the second allele. In some embodiments of the method, the RNA-guided endonuclease is a Cas9 or Cpf1 endonuclease.

In other aspects, the disclosure provides a method of selecting members of a library having a phenotype of interest, the method comprising: (a) providing a plant or seed library of any one of the above-mentioned embodiments or any other embodiment provided herein, (b) selecting at least one member of the library that exhibits a phenotype of interest, and (c) crossing the at least one member to at least one plant that does not contain the expression cassette. In some embodiments, the method further comprises propagating or multiplying the plant obtained in step (c). In some embodiments, the method further comprises producing a seed from the plant obtained in step (c).

In some aspects, the disclosure provides a plant or seed obtainable, or obtained by, the method of any one of the methods described above or otherwise herein.

In other aspects, the disclosure provides a plant library comprising a plurality of F1 hybrid plants obtainable, or obtained by, a process comprising (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, and (c) crossing the first plant to the second plant to produce a plurality of F1 hybrid plants, each F1 hybrid plant in the plurality comprising the first allele, the second allele and the expression cassette. In some embodiments, the first plant is hemizygous for the expression cassette. In some embodiments, the first plant is homozygous for the first allele and the second plant is homozygous for the second allele. In some embodiments, the method further comprises maintaining the plurality of F1 hybrid plants or F1 hybrid seeds under conditions that permit the gRNA/Cas9 to induce mutations within the target region of the second allele. In some embodiments, the RNA-guided endonuclease is a Cas9 or Cpf1 endonuclease.

In some aspects, the disclosure provides a seed library comprising a plurality of F1 hybrid seeds obtainable, or obtained by, a process comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, and (c) crossing the first plant to the second plant to produce a plurality of F1 hybrid seeds, each F1 hybrid seed in the plurality comprising the first allele, the second allele and the expression cassette. In some embodiments, the first plant is hemizygous for the expression cassette. In some embodiments, the first plant is homozygous for the first allele and the second plant is homozygous for the second allele. In some embodiments, the method further comprises maintaining the plurality of F1 hybrid plants or F1 hybrid seeds under conditions that permit the gRNA/Cas9 to induce mutations within the target region of the second allele. In some embodiments, the RNA-guided endonuclease is a Cas9 or Cpf1 endonuclease.

In other aspects, the disclosure provides a method of producing a plant or seed, the method comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, (c) crossing the first plant to the second plant to produce a plurality of F1 hybrid plants, each F1 hybrid plant in the plurality comprising the first allele, the second allele and the expression cassette, (d) maintaining the plurality of F1 hybrid plants under conditions that permit the gRNA/RNA-guided endonuclease to induce mutations within the target region of the second allele, (e) selecting an F1 hybrid plant of step (d) having a phenotype of interest, and (f) performing a cross with the F1 hybrid plant to produce a progeny plant or seed containing at least one gRNA/RNA-guided endonuclease-induced mutation. In some embodiments, the mutation is a deletion, inversion, translocation or insertion, or a combination of structural variations thereof. In some embodiments, the method further comprises propagating or multiplying the progeny plant or seed. In some embodiments, the method further comprises producing a seed from the progeny plant or seed. In some embodiments, the RNA-guided endonuclease is a Cas9 or Cpf1 endonuclease.

In some aspects, the disclosure provides a plant or seed that is homozygous for a second allele of a gene of interest containing at least one gRNA/RNA-guided endonuclease-induced mutation obtainable, or obtained by, a process comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, (c) crossing the first plant to the second plant to produce a plurality of F1 hybrid plants, each F1 hybrid plant in the plurality comprising the first allele, the second allele and the expression cassette, (d) maintaining the plurality of F1 hybrid plants under conditions that permit the gRNA/RNA-guided endonuclease to induce mutations within the target region of the second allele, (e) selecting an F1 hybrid plant of step (d) having a phenotype of interest, and (f) performing a cross with the F1 hybrid plant to produce a progeny plant or seed that is homozygous for the second allele containing at least one gRNA/RNA-guided endonuclease-induced mutation. In some embodiments, the mutation is a deletion, inversion, translocation or insertion, or a combination of structural variations thereof.

In other aspects, the disclosure provides a plant cell or seed cell obtainable, or obtained by, a process comprising isolating a cell from the plant or seed of any one of the embodiments described above or otherwise herein.

In some aspects, the disclosure provides an isolated DNA molecule comprising a second allele of a gene of interest containing at least one gRNA/Cas9-induced mutation or a fragment of the second allele containing the target region containing the at least one gRNA/Cas9-induced mutation, the DNA molecule obtainable, or obtained by, a process comprising isolating a DNA molecule comprising the second allele, or the fragment thereof, from the plant or seed of any one of the embodiments described above or otherwise herein or from the plant cell or seed cell of any one of the embodiments described above or otherwise herein.

In other aspects, the disclosure provides a method of producing a plant or seed, the method comprising: (a) providing a first plant comprising (i) a gene of interest comprising a coding sequence and having a first allele that is a hypomorphic allele or a null allele, and (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest, (b) providing a second plant comprising the second allele of the gene of interest, (c) crossing the first plant to the second plant to produce a plurality of F1 hybrid plants, each F1 hybrid plant in the plurality comprising the first allele, the second allele and the expression cassette, (d) maintaining the plurality of F1 hybrid plants under conditions that permit the gRNA/RNA-guided endonuclease to induce mutations within the target region of the second allele, (e) selecting an F1 hybrid plant of step (d) having a phenotype of interest, and (f) performing a cross with the F1 hybrid plant to produce a progeny plant or seed that is homozygous for the second allele containing at least one gRNA/RNA-guided endonuclease-induced mutation. In some embodiments, the method further comprises propagating or multiplying the progeny plant or seed. In some embodiments, the method further comprises producing a seed from the progeny plant or seed. In some embodiments, the method further comprises isolating a cell from the plant or seed. In some embodiments, the method further comprises isolating a DNA molecule from the cell, wherein the isolated DNA molecule comprises the second allele of the gene of interest containing the at least one gRNA/Cas9-induced mutation or a fragment of the second allele containing the target region containing the at least one gRNA/Cas9-induced mutation. In some embodiments, the RNA-guided endonuclease is a Cas9 or Cpf1 endonuclease.

In some aspects, the disclosure provides a nucleic acid comprising an expression construct encoding a RNA-guided endonuclease and at least four different guide RNAs (gRNAs), each gRNA containing a sequence that is complementary to a target sequence within a target region in an allele of a gene of interest in a plant, wherein the target region is 0 to 5000 base pairs upstream of the 5′ end of the coding sequence of the gene of interest or wherein the target region is 0 to 5000 base pairs downstream of the 3′ end of the coding sequence of the gene of interest. In some embodiments, the target region comprises a regulatory region of the gene of interest. In some embodiments, the regulatory region comprises a transcription factor binding site, an RNA polymerase binding site, a TATA box, or a combination thereof. In some embodiments, the regulatory region is a promoter. In some embodiments, the expression cassette encodes at least five different gRNAs. In some embodiments, the expression cassette encodes at least six different gRNAs. In some embodiments, the expression cassette encodes at least seven different gRNAs. In some embodiments, the expression cassette encodes at least eight different gRNAs. In some embodiments, the expression cassette encodes four to nine different gRNAs. In some embodiments, the expression cassette encodes five to eight different gRNAs. In some embodiments, the expression cassette encodes six to eight different gRNAs. In some embodiments, each target sequence is located 50 to 500 base pairs away from at least one other target sequence. In some embodiments, the expression cassette contains a constitutive promoter. In some embodiments, the nucleic acid is a vector. In some embodiments, the plant is a crop plant. In some embodiments, the nucleic acid is contained within a cell. In some embodiments, the cell is a plant cell. In some embodiments, the cell is a bacterial cell. In some embodiments, the RNA-guided endonuclease is a Cas9 or Cpf1 endonuclease.

In other aspects, the disclosure provides use of the library of any one of the embodiments described above or otherwise herein, the DNA molecule of any one of the embodiments described above or otherwise herein, the nucleic acid of any one of the embodiments described above or otherwise herein, or the F1 hybrid plant of any one of the embodiments described above or otherwise herein for the production of a crop plant or seed thereof. In some embodiments, the crop plant or seed thereof carries a mutation in the regulatory region of a gene that controls a commercially relevant trait. In some embodiments, the crop plant or seed thereof is transgene-free.

In some aspects, the disclosure provides a method for generating crop plants or a seed thereof with alleles that weakly affect one or more commercially relevant traits, comprising the use of the library of any one of the embodiments described above or otherwise herein, the DNA molecule of any one of the embodiments described above or otherwise herein, the nucleic acid of any one of the embodiments described above or otherwise herein, or the F1 hybrid plant of any one of the embodiments described above or otherwise herein. In some embodiments, the commercially relevant trait is a yield-related trait or a quality-related trait.

In other aspects, the disclosure provides a crop plant or seed thereof obtainable or obtained by the use or method of any one of the embodiments described above or otherwise herein.

In some aspects, the disclosure provides a method of generating a commercially relevant allele or trait that can be used in plant breeding, comprising (a) selecting an F1 hybrid plant, which is hemizygous for an expression cassette that encodes a RNA-guided endonuclease and at least two different gRNAs, each gRNA containing a sequence that is complementary to a target sequence within a target region of a gene of interest, and having a first allele of the gene of interest that is a null allele or a hypomorphic allele and a second allele of the gene of interest carrying a gRNA/endonuclease-induced mutation within the promotor region of the gene of interest; and (b) fixing the second allele in a plant to produce a progeny plant or seed that is homozygous for that second allele. In some embodiments, the expression cassette encodes a Cas9 or Cpf1 endonuclease. In some embodiments, the second allele is fixed in a progeny plant or seed by performing a self-cross of the F1 hybrid plant. In some embodiments, the progeny plant or seed does not carry the expression cassette. In some embodiments, the second allele is fixed in a progeny plant or seed by performing at least two outcrosses of the F1 hybrid plant with a plant that does not contain the expression cassette. In some embodiments, the F1 hybrid plant is a crop plant. In some embodiments, after step (b), the second allele is introduced into a different plant that does not contain the expression cassette to produce a different plant or seed containing the second allele, and optionally further propagating or multiplying the different plant or seed containing the second allele. In some embodiments, the second allele is fixed in the different plant or seed, for the production of a plant or seed that is homozygous for the second allele.

In other aspects, the disclosure provides a method for producing a crop plant or crop seed having a commercially relevant allele of a gene of interest, comprising using the method of any one of the embodiments described above or otherwise herein to produce a commercially relevant allele of a gene of interest, introducing the allele into a crop plant, to produce a crop plant or crop seed containing the allele, and optionally further propagating or multiplying that crop plant or crop seed.

In some aspects, the disclosure provides a method of generating a commercially relevant allele or trait that can be used in plant breeding, comprising (a) selecting an F1 hybrid plant, which is hemizygous for an expression cassette that encodes a RNA guided endonuclease and at least two different gRNAs, each gRNA containing a sequence that is complementary to a target sequence within a target region of a gene of interest, and having a first allele of the gene of interest that is a null allele or a hypomorphic allele and a second allele of that gene carrying a gRNA/endonuclease induced mutation within the promotor region of that gene; and (b) performing a cross of the F1 hybrid plant to produce a progeny plant or seed that is heterozygous for that second allele. In some embodiments, the expression cassette encodes a Cas9 or Cpf1 endonuclease. In some embodiments, the cross of the F1 hybrid plant is a self-cross. In some embodiments, the cross of the F1 hybrid plant is an outcross. In some embodiments, the progeny plant does not carry the expression cassette. In some embodiments, the F1 hybrid plant is a crop plant. In some embodiments, after producing the progeny plant or seed that is heterozygous for the second allele, the second allele is introduced into a different plant that does not contain the expression cassette for the production of a plant or seed, optionally further propagating or multiplying that plant or seed. In some embodiments, the second allele is fixed in the different plant, for the production of a plant or seed that is homozygous for the second allele.

In other aspects, the disclosure provides a method for producing a crop plant or crop seed having a commercially relevant allele of a gene of interest, comprising using the method of any one of the embodiments described above or otherwise herein to produce a commercially relevant allele of a gene of interest, introducing the allele into a crop plant, to produce a crop plant or crop seed containing the allele, and optionally further propagating or multiplying that crop plant or crop seed.

In some aspects, the disclosure provides a plant library comprising a plurality of F1 hybrid plants, each F1 hybrid plant in the plurality comprising: (a) a gene of interest comprising a coding sequence and having a first allele that is a hypomorphic allele or a null allele and a second allele that is different from the first allele, and (b) a CRISPR/Cas9 expression cassette that encodes a Cas9 endonuclease and at least four different guide RNAs (gRNAs), each gRNA containing a sequence that is complementary to a target sequence within a target region in the second allele of the gene of interest, wherein the target region is 0 to 5000 base pairs upstream of the 5′ end of the coding sequence of the gene of interest or wherein the target region is 0 to 2000 base pairs downstream of the 3′ end of the coding sequence of the gene of interest.

In some aspects, the disclosure provides a seed library comprising a plurality of F1 hybrid seeds, each F1 hybrid seed in the plurality comprising: (a) a gene of interest comprising a coding sequence and having a first allele that is a hypomorphic allele or a null allele and a second allele that is different from the first allele, and (b) a CRISPR/Cas9 expression cassette that encodes a Cas9 endonuclease and at least four different guide RNAs (gRNAs), each gRNA containing a sequence that is complementary to a target sequence within a target region in the second allele of the gene of interest, wherein the target region is 0 to 5000 base pairs upstream of the 5′ end of the coding sequence of the gene of interest or wherein the target region 0 to 2000 base pairs downstream of the 3′ end of the coding sequence of the gene of interest.

In some embodiments of the plant library or seed library, the target region comprises a regulatory region of the gene of interest. In some embodiments of the plant library or seed library, the regulatory region comprises a transcription factor binding site, an RNA polymerase binding site, a TATA box, or a combination thereof. In some embodiments of the plant library or seed library, the regulatory region is a promoter. In some embodiments of the plant library or seed library, the CRISPR/Cas9 expression cassette encodes at least five different gRNAs. In some embodiments of the plant library or seed library, the CRISPR/Cas9 expression cassette encodes at least six different gRNAs. In some embodiments of the plant library or seed library, the CRISPR/Cas9 expression cassette encodes at least seven different gRNAs. In some embodiments of the plant library or seed library, the CRISPR/Cas9 expression cassette encodes at least eight different gRNAs. In some embodiments of the plant library or seed library, the CRISPR/Cas9 expression cassette encodes four to nine different gRNAs. In some embodiments of the plant library or seed library, the CRISPR/Cas9 expression cassette encodes five to eight different gRNAs. In some embodiments of the plant library or seed library, the CRISPR/Cas9 expression cassette encodes six to eight different gRNAs. In some embodiments of the plant library or seed library, the second allele is a naturally-occurring allele. In some embodiments of the plant library or seed library, the second allele is not a hypomorphic allele. In some embodiments of the plant library or seed library, the second allele is not a null allele. In some embodiments of the plant library or seed library, the first allele contains a mutation in a regulatory region of the gene of interest. In some embodiments of the plant library or seed library, the first allele contains a mutation in a coding sequence of the gene of interest. In some embodiments of the plant library or seed library, the first allele is a hypomorphic allele that results in an mRNA expression level of the gene of interest that is at least 70% lower than an allele of the gene of interest that does not contain the mutation. In some embodiments of the plant library or seed library, each gRNA is a single-guide RNA (sgRNA). In some embodiments of the plant library or seed library, each target sequence is located 200 to 500 base pairs away from at least one other target sequence. In some embodiments of the plant library or seed library, the library contains at least 50 members. In some embodiments of the plant library or seed library, the plant or seed is a crop plant or crop seed. In some embodiments of the plant library or seed library, the library is a seed or plant library and at least one member of the library contains a gRNA/Cas9-induced mutation in the second allele. In some embodiments of the plant library or seed library, the gRNA/Cas9-induced mutation is a deletion, inversion, translocation or insertion, or a combination of structural variations thereof.

Other aspects of the disclosure relate to a method of generating a plant library comprising a plurality of F1 hybrid plants, the method comprising: (a) providing a first plant comprising (i) a gene of interest comprising a coding sequence and having a first allele that is a hypomorphic allele or a null allele, and (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, and (c) crossing the first plant to the second plant to produce a plurality of F1 hybrid plants, each F1 hybrid plant in the plurality comprising the first allele, the second allele and the CRISPR/Cas9 expression cassette.

Other aspects of the disclosure relate to a method of generating a seed library comprising a plurality of F1 hybrid seeds, the method comprising: (a) providing a first plant comprising (i) a gene of interest comprising a coding sequence and having a first allele that is a hypomorphic allele or a null allele, and (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, and (c) crossing the first plant to the second plant to produce a plurality of F1 hybrid seeds, each F1 hybrid seed in the plurality comprising the first allele, the second allele and the CRISPR/Cas9 expression cassette.

In some embodiments of the method of generating a plant library or a seed library, the first plant is hemizygous for the CRISPR/Cas9 expression cassette. In some embodiments of the method of generating a plant library or a seed library, the first plant is homozygous for the first allele and the second plant is homozygous for the second allele. In some embodiments of the method of generating a plant library or a seed library, the method further comprises maintaining the plurality of F1 hybrid plants or F1 hybrid seeds under conditions that permit the gRNA/Cas9 to induce mutations within the target region of the second allele. In some embodiments of the method of generating a plant library or a seed library, each gRNA is a single-guide RNA (sgRNA).

In other aspects, the disclosure provides a method of selecting members of a library having a phenotype of interest, the method comprising: (a) providing a plant or seed library of any one of the above-mentioned embodiments or any other embodiment described herein, (b) selecting at least one member of the library that exhibits a phenotype of interest, and (c) crossing the at least one member to at least one plant that does not contain the CRISPR/Cas9 expression cassette.

In yet other aspects, the disclosure provides a plant or seed obtainable, or obtained by, any one of the methods described above or otherwise herein.

In other aspects, the disclosure provides a plant library comprising a plurality of F1 hybrid plants obtainable, or obtained by, a process comprising: (a) providing a first plant comprising (i) a gene of interest comprising a coding sequence and having a first allele that is a hypomorphic allele or a null allele, and (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, and (c) crossing the first plant to the second plant to produce a plurality of F1 hybrid plants, each F1 hybrid plant in the plurality comprising the first allele, the second allele and the CRISPR/Cas9 expression cassette.

In other aspects, the disclosure provides a seed library comprising a plurality of F1 hybrid seeds obtainable, or obtained by, a process comprising: (a) providing a first plant comprising (i) a gene of interest comprising a coding sequence and having a first allele that is a hypomorphic allele or a null allele, and (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, and (c) crossing the first plant to the second plant to produce a plurality of F1 hybrid seeds, each F1 hybrid seed in the plurality comprising the first allele, the second allele and the CRISPR/Cas9 expression cassette.

In some embodiments of the plant library or seed library, the first plant is hemizygous for the CRISPR/Cas9 expression cassette. In some embodiments of the plant library or seed library, the first plant is homozygous for the first allele and the second plant is homozygous for the second allele. In some embodiments of the plant library or seed library, the process further comprises maintaining the plurality of F1 hybrid plants or F1 hybrid seeds under conditions that permit the gRNA/Cas9 to induce mutations within the target region of the second allele. In some embodiments of the plant library or seed library, each gRNA is a single-guide RNA (sgRNA).

In another aspect, the disclosure provides a plant or seed that is homozygous for a second allele of a gene of interest containing at least one gRNA/Cas9-induced mutation obtainable, or obtained by, a process comprising: (a) providing a first plant comprising (i) a gene of interest comprising a coding sequence and having a first allele that is a hypomorphic allele or a null allele, and (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest, (b) providing a second plant comprising the second allele of the gene of interest, (c) crossing the first plant to the second plant to produce a plurality of F1 hybrid plants, each F1 hybrid plant in the plurality comprising the first allele, the second allele and the CRISPR/Cas9 expression cassette, (d) maintaining the plurality of F1 hybrid plants under conditions that permit the gRNA/Cas9 to induce mutations within the target region of the second allele, (e) selecting an F1 hybrid plant of step (d) having a phenotype of interest, and (f) performing a cross with the selected F1 hybrid plant to produce a progeny plant or seed that is homozygous for the second allele containing at least one gRNA/Cas9-induced mutation.

In some embodiments of the plant or seed, the mutation is a deletion, inversion, translocation or insertion, or a combination of structural variations thereof.

Yet other aspects of the disclosure relate to a plant cell or seed cell obtainable, or obtained by, a process comprising isolating a cell from a plant or seed as described herein.

Yet other aspects of the disclosure relate to an isolated DNA molecule comprising a second allele of a gene of interest containing at least one gRNA/Cas9-induced mutation or a fragment of the second allele containing the target region containing the at least one gRNA/Cas9-induced mutation, the DNA molecule obtainable, or obtained by, a process comprising isolating a DNA molecule comprising the second allele, or the fragment thereof, from a plant or seed as described herein or from the plant cell or seed cell as described herein.

### BRIEF DESCRIPTION OF THE DRAWINGS

FIGS. 1A-1E show an example of the process of generating quantitative mutational and, as a result, phenotypic variation using CRISPR/Cas9 editing. FIG. 1A is a diagram that shows generation of F1 progeny by crossing a strong promoter mutant containing the Cas9 construct with a wild-type allele containing a wild-type promoter. FIGS. 1B and 1C are diagrams show that in the F1 progeny new, different alleles are generated by the gRNAs/Cas9 inducing mutations in the wild-type allele, which are expected to have a variety of phenotypes from weak to strong. FIGS. 1D and 1E are diagrams that show a Punnett square for the F2 progeny that would be generated by self-crossing a plant containing an allele of interest from the F1 generation. As shown in FIG. 1E, it is expected that approximately 1:16 of the F2 progeny will contain the new allele of interest without the Cas9 construct.

FIGS. 2A-2F show engineering of a Quantitative Trait Locus (QTL) by CRISPR-Cas9 in tomato. FIG. 2A is a diagram showing that selection for increasing fruit size has driven domestication and breeding in tomato. FIG. 2B is a diagram and photograph showing a genetic circuit controlling stem cell homeostasis is regulated by CLV3 and WUS. FIG. 2C is a diagram showing that CRISPR-Cas9 targeting the region downstream of WUS containing the lc motif in S.pim and S.lyc disrupted a putative AGAMOUS binding site (CArG). Black arrowheads, sgRNAs. FIG. 2D is a series of photographs showing that lc^{CR} lines showed increase locule number in fruits in both S.pim and S.lyc. FIG. 2E and FIG. 2F are bar graphs showing that a quantitative shift in locule number was observed in lc^{CR} lines, and was synergistic with fas in both S.pim and S.lyc. Data are shown as percentages within each category. N/n, number of plants and flowers/fruits counted per genotype. Two-tailed t-test was applied and P values are shown. Bars, 1 cm (FIG. 2A, D) and 100 μm (FIG. 2B).

FIGS. 3A-3K show robust and efficient promoter targeting in SlCLV3 by CRISPR-Cas9 produced quantitative effects on floral organ number and fruit size. FIG. 3A is a series of photographs and a diagram showing that fas and clv3^{CR} cover a limited spectrum of floral organ number and fruit size changes, and quantitative effects could be achieved by modulating CLV3 expression. FIG. 3B is a diagram showing that the promoter of SlCLV3 was targeted by CRISPR-Cas9 using 8 sgRNAs (arrowheads). Black arrows, primers used for PCR and genotyping. FIG. 3C is a photograph of PCR screening that showed deletions of different sizes in 4 out of 6 T0 plants. FIG. 3D is a series of photographs showing that floral morphology and fruit size differences were seen among T0 lines. FIG. 3E is a bar graph showing that quantitative effects, different from WT, fas and clv3^{CR} were observed in floral organ number among T0 plants. Data are shown as mean±s.d. from at least 10 flowers per line. FIG. 3F is a diagram showing results of Sanger sequencing. which was performed for all T0-derived PCR products. Insertions and deletions are indicated as numbers or letters. T0-5 and T0-6 only contained wild-type (WT) alleles. FIG. 3G is a series of photographs showing PCR-based genotyping in 24 plants from T0-1 and T0-2 progeny, with a quarter carrying a non-amplifiable allele. FIG. 3H is a diagram demonstrating that genome sequencing of T0-1 and T0-2 offspring homozygous for non-amplifiable alleles, showed duplication of the entire target region and translocation segments from different genomic sites and a 7.3 kb deletion, respectively. FIG. 3I is a bar graph showing floral organ number quantification of stable homozygous plants for 4 alleles from T0-1 and T0-2. Black arrowheads, WT values. Data are shown as means±s.d. for at least 3 individuals per line FIG. 3J is a bar graph showing that a 20% increased 2 locule category was observed in SlCLV3^{CR-pro1-2} compared to WT. FIG. 3K is a bar graph showing CLV3 and WUS expression in WT, clv3^{CR} and 4 alleles derived from T0-1 and T0-2 progeny determined by qRT-PCR, normalized to UBI expression in meristems at the transition stage. Data are shown as means±s.e. of two independent biological replicates per genotype and 3 technical replicates each. Bars, 100 μm and 1 cm (FIG. 3A), 1 cm (FIG. 3D).

FIGS. 4A-4I show production of a population containing new alleles for SlCLV3 with quantitative effects in locule number. FIG. 4A is a diagram showing that a sensitized F1 population was generated by crossing T0-2 as male to WT. Hemizygous Cas9 individuals highlighted in bold and by a dotted square. FIG. 4B is a diagram showing that F1 transgenic plants are expected to produce new alleles from CRISPR-Cas9-mediated targeting of the wild type allele. FIG. 4C is a bar graph showing that F1 plants were clustered into 3 categories, with ˜25% of the total population showing quantitative increase in locule number. Data are shown as percentages, including the number of plants per category. FIG. 4D is a series of photographs of a PCR-based screen for generated alleles in F1 categories strong and moderate. Black arrow, PCR product of allele SlCLV3^{CR-pro2-1}; lower panel, PCR genotyping for SlCLV3^{CR-pro2-2} FIG. 4E is a diagram of a Punnett square depicting expected segregation in F1 populations for both Cas9 and SlCLV3^{CR-pro} alleles. Black asterisk, new allele. FIG. 4F is a photograph showing segregation for SlCLV3^{CR-pro} and Cas9 in 32 SlCLV3^{CR-pro2-1/7} F2 individuals. Black arrowhead, non-transgenic SlCLV3^{CR-pro7/7} homozygous individuals. FIG. 4G is a diagram of results of Sanger sequencing that was performed in 14 F2 populations to characterize lesions present in each allele. Insertions and deletions indicated as numbers or letters. FIG. 4H Is a diagram of the quantification of locule number for each allele performed in F3 families. Line with arrows indicates similar phenotypic values for SlCLV3^{CR-pro-5} and fas. Data are shown as percentages within each category from at least 4 individuals, including mean±s.d. FIG. 4I is a diagram showing CLV3 and WUS expression in WT, fas and 14 alleles derived from moderate and strong categories determined by qRT-PCR, normalized to UBI expression in meristems at the transition stage. Data are shown as means±s.e. of two independent biological replicates per genotype and 3 technical replicates each.

FIGS. 5A-5D show that promoter targeting in SP led to quantitative effects in sympodial shoot flowering. FIG. 5A is a diagram and photograph showing that upstream regulatory regions of SP were targeted by CRISPR-Cas9 using 8 sgRNAs (arrowheads). Black arrows, primers used for PCR and genotyping. PCR-based screen showed deletions with different sizes in all T0 plants obtained. FIG. 5B is a diagram of the results of Sanger sequencing that was performed for all T0-derived PCR products. Indel sizes indicated as numbers or letters. FIG. 5C is a series of photographs of representative main shoots from WT, sp and 3 SP^{CR-pro} mutants. Gray arrowheads, inflorescences. FIG. 5D is a bar graph showing quantification of flowering time from five successive sympodial shoots in WT, sp and 3 SP^{CR-pro} mutants. Two-tailed t-test was applied and P values are shown. Bars, 5 cm (D).

FIG. 6 shows a diagram of CRISPR-Cas9-generated mutations in (A) the promoter of ZmCLE7 and (b) the promoter of ZmFCP1 in maize. The black line (pFCP1-Ref) shows the promoter region and the locations of each sgRNA target site (triangles).

FIG. 7 shows an annotated CRISPR/Cas9 construct encoding a Cas9 protein and 8 single-guide RNAs (sgRNAs) that target sites within a region of 2000 bp upstream of the transcriptional start site (TSS) of SlCLV3 (Solyc11g071380). The sequence is SEQ ID NO: 2.

### SEQUENCES

Below is a brief description of certain sequences described herein.

SEQ ID NO: 1 is an example Cas9 endonuclease amino acid sequence.

SEQ ID NO: 2 is an example CRISPR/Cas9 construct encoding a Cas9 protein and 8 single-guide RNAs (sgRNAs) that target sites within a region of 2000 bp upstream of the transcriptional start site (TSS) of SlCLV3 (Solyc11g071380).

SEQ ID NO: 3 is an example CRISPR/Cas9 construct encoding a Cas9 protein and 8 sgRNAs that target sites within a region upstream of the transcriptional start site (TSS) of SP.

SEQ ID NO: 4 is an example ZmCLE7 promoter CRISPR sgRNA array containing 9 sgRNAs.

SEQ ID NO: 5 is an example ZmFCP1 promoter CRISPR sgRNA array containing 9 sgRNAs.

### DETAILED DESCRIPTION

Improving traits such as yield and quality remains a top priority for plant growers, especially for growers who produce crop plants. Traditionally, plants having improved traits have been identified by chemical or physical introduction of mutations genome-wide and screening such genetically-altered plants for improved traits. More recently, technologies such as CRISPR (clustered regularly interspaced short palindromic repeats)/Cas9 through deletion of all or a portion of a coding sequence. However, such null alleles, can drastically affect the phenotype of a plant resulting in undesirable traits such as sterility.

In contrast, weak alleles that retain some level of functionality of the underlying wild-type gene can improve some traits in the plants but avoid introducing other unexpected or undesirable traits. The results disclosed herein demonstrate that targeting regulatory regions such as promoters for mutagenesis can generate a high frequency of such useful weak alleles. To date, generating weak alleles has been a time-consuming process that requires either precise identification of regulatory regions for mutagenesis or screening of genome-wide mutations for phenotypes that may be caused by a weak allele and sequencing of those plants. Identification of weak alleles is further complicated by the fact that weak alleles may have subtle phenotypes that are difficult or impossible to detect in certain backgrounds, such as when the plant is heterozygous and the other allele of the gene is wild-type or when the plant is homozygous for the weak allele but there is some functional redundancy with another gene or genes. Further complicating the generation of weak alleles is the fact that the precise location and causative variants for the many Quantitative Trait Loci (QTL) that map to regulatory regions are largely unknown. Moreover, the modular organization and inherent redundancy of cis-regulatory motifs in regulatory regions makes it challenging to predict useful targets within regulatory regions for a gene of interest.

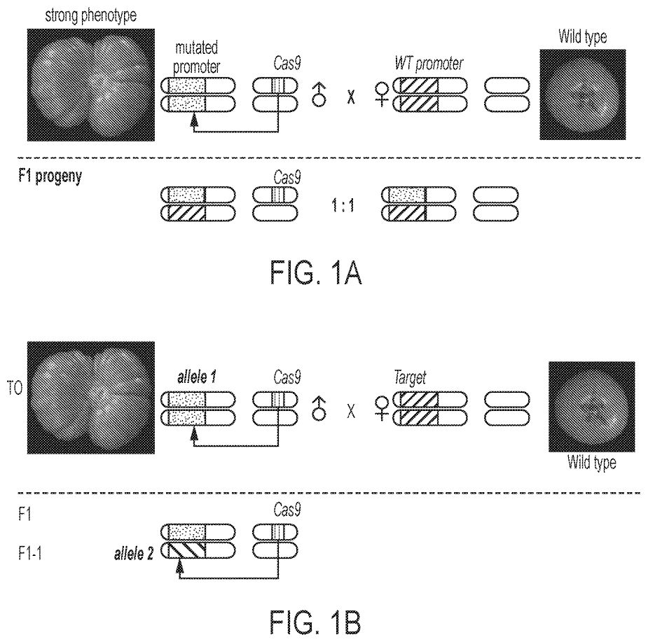

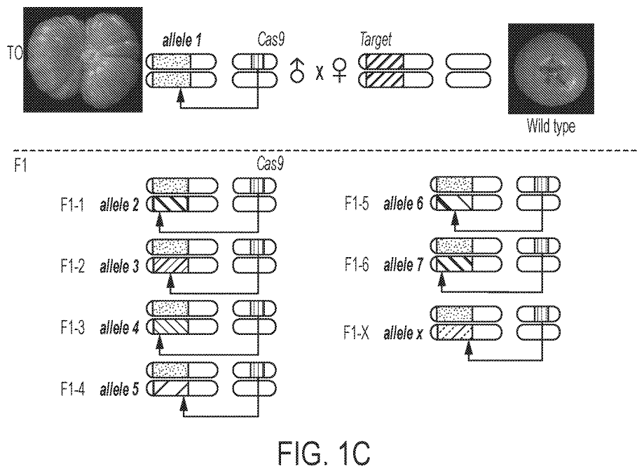

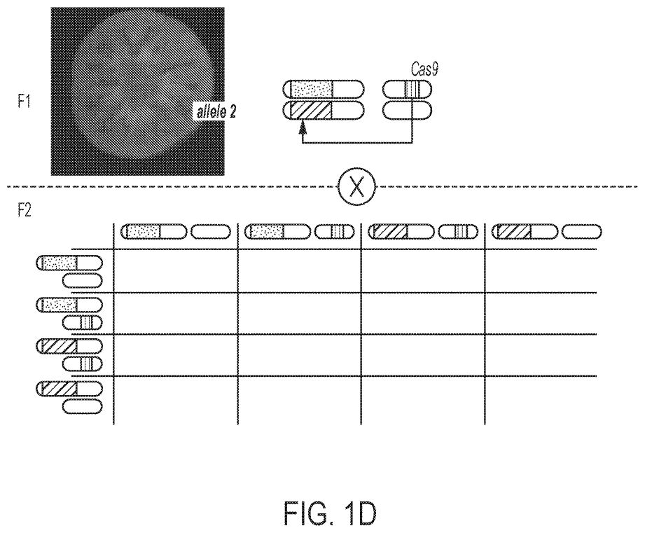

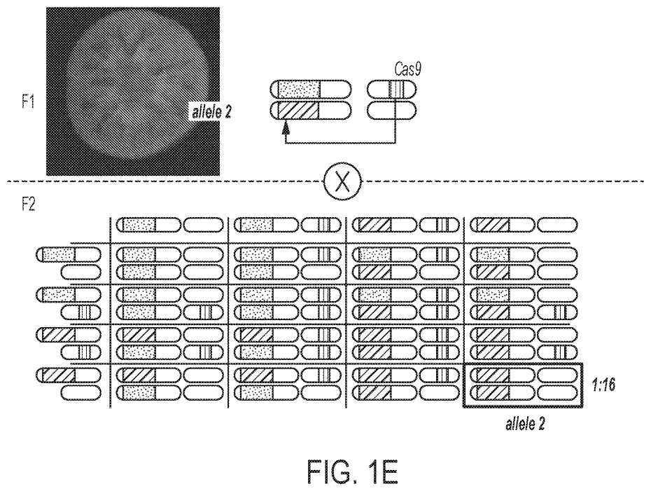

However, as described herein, these same properties of regulatory regions can be exploited to create alleles that provide quantitative variation. In one embodiment, such alleles can be generated by inducing random mutations in regulatory regions to create enough genetic variation to induce useful transcriptional changes that result in phenotypic variation. For example, as described herein, targeted mutagenesis of a putative regulatory region of a gene (e.g., within 5 kilobases upstream or downstream of the coding sequence) with a construct containing an RNA-guided endonuclease Cas9 and several sgRNAs that target different sequences within the regulatory region results in generation of a variety of mutations that confer a range of phenotypes. More specifically, as a non-limiting example, a CRISPR/Cas9 construct containing several different sgRNAs can be introduced into a first plant containing a strong phenotype caused by a null allele of a gene of interest (FIG. 1A). In some embodiments, the construct is integrated onto the same chromosome as the gene of interest. In other embodiments, integration of the construct onto a different chromosome than the gene of interest is preferable so that the construct can later be removed through crosses without having to undergo homologous recombination to separate the construct from the gene of interest. To that end, in some embodiments, it is also advantageous for the construct to be introduced into the first plant as a hemizygous copy so that removal of the construct can be accomplished through a single cross. This first plant may then be crossed to a second plant containing a wild-type allele of the same gene of interest to create a sensitized F1 population in which each plant will contain the null allele and approximately half will be hemizygous for the RNA-guided endonuclease (e.g., Cas9) construct (FIG. 1A). Within the F1 population, gRNA/RNA-guided-endonuclease-induced mutations occur in the wild-type allele of the gene of interest (FIG. 1B) and, due the random combinations of the activities of the different gRNAs within each plant, are expected to generate a variety of mutations creating a variety of new alleles of the gene of interest (FIG. 1C). F1 plants may then be screened for the phenotype of interest. Each F1 plant identified as having a phenotype of interest may then be self-crossed (FIG. 1D) to create an F2 population in which approximately 1 in 16 plants will contain the new allele in the absence of the CRISPR/RNA-guided endonuclease (e.g., CRISPR/Cas9) construct (FIG. 1E). Advantageously, because a variety of mutations are introduced in the F1 population, it is not necessary to precisely identify the location of active subsequences (e.g., transcription factor binding sites) of the regulatory region as the mutational diversity is likely to result in at least some percentage of plants having a mutation within such active subsequences. As a result, libraries of plants containing various regulatory region mutations can be created and screened for a variety of phenotypes, either alone or in combination, such as increased yield or quality.

In addition, these libraries can be created and used to identify new weak alleles, e.g., by (a) performing direct introduction of a construct containing an RNA-guided endonuclease (e.g., Cas9) and several sgRNAs into a heterozygous hypomorphic or null allele background or (b) outcrossing to wild type transgenic plants carrying a construct containing RNA-guided endonuclease (e.g., Cas9) and several sgRNAs that may also carry a hypomorphic or null allele, thereby expanding both the number of individuals that comprise a library and the number of alleles with weak effects that can be screened for a variety of phenotypes, such as increased yield, quality or both. As described above, this sensitized heterozygous mutant background allows for the identification of weak alleles that would otherwise be difficult or impossible to detect due to subtle phenotypes generally associated with weakly penetrant mutations.

This approach allows for immediate selection and fixation of novel, useful alleles in transgene-free plants. For example, through rapid generation of plant and seed libraries carrying such novel alleles, this technology allows for practical expansion and enhancement of quantitative, phenotypic variation in a diverse range of traits in a wide variety of commercially relevant plants. For example, in some embodiments, the weak alleles as described herein, the target region as described herein, or the gRNA/RNA-guided-endonuclease-mediated mutations in the target region may be introduced or transferred to another plant or seed by any method described herein or known to those of skill in the art. Accordingly, the disclosure provides in part libraries, methods of generating libraries, and constructs (e.g., CRISPR/RNA-guided endonuclease constructs (e.g., CRISPR/Cas9 constructs)) for generating weak alleles that, as exemplified herein, can enable fine-tuning of commercially relevant traits of interest in plants where such fine-tuning before was either impossible or impractical.

### Libraries

In some aspects, the disclosure provides libraries containing a plurality of plants or seeds. In some embodiments, each member of the plurality of plants or seeds contains a gene of interest comprising a coding sequence and has a first allele of the gene of interest and a second allele of the gene of interest that is different from the first allele.

In some embodiments, members of the plurality contain an expression cassette that encodes an RNA-guided endonuclease and at least two (e.g., four to eight or four to nine) guide RNAs. RNA-guided endonucleases include, e.g., Cas endonucleases such as Cas9, Cpf1 and Csm1, as well as variants thereof. In some embodiments, members of the plurality contain an expression cassette that encodes an RNA-guided endonuclease such as a Cas endonuclease (e.g., Cas9, Cpf1, or Csm1 or a functional variant thereof) and at least two (e.g., four to eight or four to nine) guide RNAs. CRISPR (clustered regularly interspaced short palindromic repeats)/Cas9 is a prokaryotic antiviral system that has been modified in order to allow for genomic engineering in many cell types (see, e.g., Sander et al. CRISPR-Cas systems from editing, regulating and targeting genomes. Nature Biotech (2014) 32: 347-355 and Hsu et al. Development and applications of CRISPR-Cas9 for genome engineering. Cell (2014) 157(6):1262-78), including in plants (see, e.g., Brooks et al. Efficient gene editing in tomato in the first generation using the clustered regularly interspaced short palindromic repeats/CRISPR-associated9 system. Plant Phys (2014) 166(3):1292-1297; Zhou et al. Large chromosomal deletions and heritable small genetic changes induced by CRISPR/Cas9 in rice. Nucleic Acids Res. (2014) 42(17):10903-10914; Feng et al. Multigeneration analysis reveals the inheritance, specificity, and patterns of CRISPR/Cas-induced gene modifications in Arabidopsis. PNAS (2014) 111(12):4632-4637 and Samanta et al. CRISPR/Cas9: an advanced tool for editing plant genomes. Transgenic Res (2016) 25:561). CRISPR/Cpf1 is another CRISPR/Cas system that may be used for genomic engineering (see, e.g., Zetsche et al. Cpf1 Is a Single RNA-Guided Endonuclease of a Class 2 CRISPR-Cas System. Cell. 2015. 163(3):759-71). CRISPR/Csm1 is yet another CRISPR system that may be used for genomic engineering (see, e.g., U.S. Pat. No. 9,896,696). Variants of RNA-guided endonucleases such as variants of Cas endonucleases may also be used, such as SpCas9-HF1 and eSpCas9 (see, e.g., Kleinstiver et al. High-fidelity CRISPR-Cas9 nucleases with no detectable genome-wide off-target effects. Nature. 2016. 529, 490-495 and Slaymaker et al. Rationally engineered Cas9 nucleases with improved specificity. Science. 2016. 351(6268):84-8). Other example variants of RNA-guided endonucleases that may be used include, but are not limited to, variants of Cpf1 endonucleases, including variants to reduce or inactivate nuclease activity, variants which further comprise at least one nuclear localization sequence, variants which further comprise at least one plastid targeting signal peptide or a signal peptide targeting Cpf1 to both plastids and mitochondria, and/or variants of Cpf1 which further comprise at least one marker domain (see, e.g., Zetsche et al. Cpf1 Is a Single RNA-Guided Endonuclease of a Class 2 CRISPR-Cas System. Cell. 2015. 163(3):759-71; U.S. Pat. No. 9,896,696). Other example variants of RNA-guided endonucleases that may be used include, but are not limited to, variants of Csm1 endonucleases, including variants to reduce or inactivate nuclease activity, variants which further comprise at least one nuclear localization sequence, variants which further comprise at least one plastid targeting signal peptide or a signal peptide targeting Cpf1 to both plastids and mitochondria, and/or variants of Cpf1 which further comprise at least one marker domain (see, e.g., U.S. Pat. No. 9,896,696). Further example RNA-guided endonucleases that may be used include, but are not limited to, LshC2c2, FnCas9, SaCas9, St1Cas9, Nmcas9, FnCpf1, AsCpf1, SpCas9-nickase, eSpcas9, Split-SpCas9, dSpCas9FokI, and SpCas9-cytidine deaminase (see, e.g., Murovec et al. New Variants of CRISPR RNA-guided genome editing enzymes. Plant Biotechnol J (2017) 15, pp. 917-926).

In some embodiments, members of the plurality of plants or seeds contain an expression cassette (e.g., a CRISPR/RNA-guided endonuclease expression cassette such as a CRISPR/Cas9 expression cassette, a CRISPR/Cpf1 expression cassette or a CRISPR/Csm1 expression cassette) that encodes a RNA-guided endonuclease (e.g., a Cas9, Cpf1 or Csm1 endonuclease) and at least two (e.g., at least 2, at least 3, at least 4, at least 5, at least 6, at least 7, at least 8 or at least 9) different guide RNAs (gRNAs), such as single-guide RNAs (sgRNAs), each gRNA (e.g., sgRNA) containing a sequence that is complementary to a target sequence within a target region. In some embodiments, the cassette contains between two and sixteen (e.g., 2, 3, 4, 5, 6, 7, 8, 9, 10, 11, 12, 13, 14, 15, or 16) different gRNAs (e.g., sgRNAs). In some embodiments, each target sequence in the target region is located 50 to 500 base pairs (e.g., 50 to 500, 50 to 400, 50 to 300, 50 to 200, 50 to 100, 100 to 500, 100 to 400, 100 to 300, 100 to 200, 200 to 500, 200 to 400, or 200 to 300 base pairs) away from at least one other different target sequence. In some embodiments, each target sequence is located next to a Protospacer Adjacent Motif (PAM) sequence, such as NGG, NAA, NNNNGATT, NNAGAA, or NAAAAC. In some embodiments, the PAM sequence is a Cpf1 or Csm1 PAM sequence, such as TTN, CTA, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, or CCGN. Guide RNA sequences, such as sgRNA sequences, can be designed using methods known in the art or described herein (see, e.g., the CRISPR tool available from crispr.mit.edu). In some embodiments, the gRNA is a single guide RNA (sgRNA) containing a trans-activating CRISPR RNA (tracrRNA) and a CRISPR RNA (crRNA) designed to cleave the target site of interest. In some embodiments, the gRNA is a sgRNA containing a crRNA. In some embodiments, the CRISPR/Cas expression cassette described herein encodes a Cas9 endonuclease, a Cpf1 endonuclease or Csm1 endonuclease or a functional variant thereof.

In some embodiments, the CRISPR/Cas expression cassette described herein encodes a Cas9 endonuclease. The Cas9 endonuclease may be any Cas9 endonuclease known in the art or described herein. In some embodiments, the Cas9 endonuclease is a rice optimized CAS9 (see, e.g., Jiang et al. Demonstration of CRISPR/Cas9/sgRNA-mediated targeted gene modification in Arabidopsis, tobacco, sorghum and rice, Nucleic Acids Res. 2013 November; 41(20):e188). In some embodiments, the Cas9 endonuclease has an amino acid sequence that is at least 90%, 95%, 98%, 99% or 100% identical to the following amino acid sequence:

(SEQ ID NO: 1)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNEVINFFKTEITLANGEIRKRPLIETNGETG
EIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKK
RKV.

In some embodiments, the CRISPR expression cassette described herein encodes a Cpf1 endonuclease. The Cpf1 endonuclease may be any Cpf1 endonuclease known in the art or described herein (e.g., FnCpf1, AsCpf1, Lb2Cpf1, CMtCpf1, MbCpf1, LbCpf1, PcCpf1, or PdCpf1, see, e.g., U.S. Pat. No. 9,896,696). In some embodiments, the CRISPR expression cassette described herein encodes a Csm1 endonuclease. The Csm1 endonuclease may be any Csm1 endonuclease known in the art or described herein (e.g., SsCsm1, SmCsm1, ObCsm1, Sm2Csm1, or MbCsm1, see, e.g., U.S. Pat. No. 9,896,696).

In some embodiments, the expression cassette (e.g., CRISPR/RNA-guided endonuclease expression cassette such as a CRISPR/Cas9 expression cassette or a CRISPR/Cpf1 expression cassette) contains a constitutive promoter, e.g., a CaMV 35s promoter, a maize U6 promoter, a rice U6 promoter, or a maize Ubiquitin promoter. In some embodiments, the expression cassette (e.g., CRISPR/RNA-guided endonuclease expression cassette such as a CRISPR/Cas9 expression cassette or a CRISPR/Cpf1 expression cassette) contains a tissue-specific promoter, e.g., an anther-specific promoter or a pollen-specific promoter (see, e.g., Unger et al. A Chimeric Ecdysone Receptor Facilitates Methoxyfenozide-Dependent Restoration of Male Fertility in Ms45 Maize. Transgenic Res 2002. 11(5), 455-465 and Twell et al. Pollen-specific gene expression in transgenic plants: coordinate regulation of two different tomato gene promoters during microsporogenesis. Development. 1990. 109(3):705-13). In some embodiments, the expression cassette (e.g., CRISPR/RNA-guided endonuclease expression cassette such as a CRISPR/Cas9 expression cassette or a CRISPR/Cpf1 expression cassette) contains an inducible promoter, e.g., an ethanol inducible promoter, a dexamethasone inducible promoter, a beta-estradiol inducible promoter, or a heat shock inducible promoter (see, e.g., Borghi. Inducible Gene Expression Systems for Plants. Methods Mol Biol. 2010. 655:65-75 and Caddick et al. An ethanol inducible gene switch for plants used to manipulate carbon metabolism. Nature Biotech. 1998. 16, 177-180). In some embodiments, the same promoter is used to drive expression of both the RNA-guided endonuclease (e.g., Cas9, Cpf1, or Csm1) sequence and the gRNA sequences. In some embodiments, different promoters are used to drive the expression of the RNA-guided endonuclease (e.g., Cas9, Cpf1, or Csm1) sequence and the gRNA sequences. In some embodiments, expression of the gRNAs is driven a using a polycistronic tRNA system (see, e.g., Xie, K, Minkenberg, B, Yang, Y. (2015). Boosting CRISPR/Cas9 multiplex editing capability with the endogenous tRNA-processing system. Proc Natl Acad Sci USA. 2015; 112: 3570-5)/

The expression cassette (e.g., CRISPR/RNA-guided endonuclease expression cassette such as a CRISPR/Cas9 expression cassette or a CRISPR/Cpf1 expression cassette) may be introduced into a plant using any method known in the art or described herein, e.g., by such as Agrobacterium-mediated recombination, viral-vector mediated recombination, microinjection, gene gun bombardment/biolistic particle delivery, or electroporation of plant protoplasts. The expression cassette (e.g., CRISPR/RNA-guided endonuclease expression cassette such as a CRISPR/Cas9 expression cassette or a CRISPR/Cpf1 expression cassette) may be integrated onto the same chromosome or a different chromosome than the gene of interest. In some embodiments, integration of the expression cassette (CRISPR/RNA-guided endonuclease expression cassette such as a CRISPR/Cas9 expression cassette or a CRISPR/Cpf1 expression cassette) onto a different chromosome than the gene of interest is preferable so that the expression cassette can later be removed through a self-cross or a cross with another plant without having to undergo homologous recombination to separate the expression cassette from the gene of interest.

In some embodiments, the second allele of the gene of interest contains the target region against which the multiple different gRNAs (e.g., sgRNAs) are designed such that mutations can be introduced into the target region of the second allele using the RNA-guided endonuclease (e.g., Cas9, Cpf1, or Csm1 endonuclease). In some embodiments, the target region or a portion thereof, is absent from the first allele. In some embodiments, the target region or a portion thereof, is present in the first allele and the second allele. In some embodiments, the first allele is a null allele in which most or the entire coding sequence is deleted such that further mutations induced by the RNA-guided endonuclease (e.g., Cas9, Cpf1, or Csm1 endonuclease) generally have no further effect on the first allele.

In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest (e.g., the second allele of the gene of interest). In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest (e.g., the second allele of the gene of interest).

In some embodiments, the target region comprises a regulatory region of the gene of interest. As used herein, a “regulatory region” of a gene of interest contains one or more nucleotide sequences that, alone or in combination, are capable of modulating expression of the gene of interest. Regulatory regions include, for example, promoters, enhancers, and introns. In some embodiments, the regulatory region comprises a transcription factor binding site, an RNA polymerase binding site, a TATA box, or a combination thereof. In some embodiments, the regulatory region is within a certain distance of the gene of interest, e.g., 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest. In some embodiments, a regulatory region may be identified using databases or other information available in the art (see, e.g. Sandelin et al 2004, Turco et al 2013, O'Connor et al 2005, Baxter et al 2012, Haudry et al 2013, Matys et al 2003, Bailey et al 2011, Korkuc et al 2014, Chia et al 2012, Sim et al 2012, Higo et al. Plant cis-acting regulatory DNA elements (PLACE) database: 1999. Nucleic Acids Res. 1999 Jan. 1; 27(1):297-300 and www.hsls.pittedu/obrc/index.php?page=URL1100876009; Plant Promoter db 3.0: ppdb.agr.gifu-u.ac.jp/ppdb/cgi-bin/index.cgi; Yilmaz et al. AGRIS: Arabidopsis Gene Regulatory Information Server, an update. Nucleic Acids Res. 2011 January, 39 (Database issue):D1118-D1122; and Lescost et al. PlantCARE, a database of plant cis-acting regulatory elements and a portal to tools for in silico analysis of promoter sequences. Nucleic Acids Res. 2002 Jan. 1; 30(1): 325-327 and bioinformatics.psb.ugent.be/webtools/plantcare/html/). In some embodiments, a regulatory region can be identified, e.g., by analyzing the sequences within a certain distance of the gene of interest (e.g., within 5 kilobases) for one or more of transcription factor binding sites, RNA polymerase binding sites, TATA boxes, reduced SNP density or conserved non-coding sequences.

Cereal crops, such as maize, in some instances have enhancer regions that are more distal than in other crops (see, e.g., Weber et al. 2016. Plant Enhancers: A Call for Discovery. Cell. Trends in Plant Science, Volume 21, Issue 11, 974-987). Accordingly, in some embodiments, if the crop is a cereal crop (such as maize), the target region may be larger, e.g., 0 to 100 kilobases (e.g., 0 to 100, 0 to 90, 0 to 80, 0 to 70, 0 to 60, 0 to 50, 0 to 40, 0 to 30, 0 to 20 or 0 to 10 kilobases) upstream of the 5′ end of the coding sequence of the gene of interest (e.g., the second allele of the gene of interest) or 0 to 60 kilobases (e.g., 0 to 60, 0 to 50, 0 to 40, 0 to 30, 0 to 20 or 0 to 10 kilobases) base pairs downstream of the 3′ end of the coding sequence of the gene of interest (e.g., the second allele of the gene of interest). Such larger regions may include both proximal promoter regions (e.g., within 1 to 3 Kb of the 5′ end of the coding sequence) and distal enhancer regions.

In some embodiments, the gene of interest is a gene that modulates a trait of interest in a plant. Traits of interest include, for example, yield-related traits and quality-related traits. Yield-related traits include, for example, product size (e.g., fruit or vegetable size), product number (e.g., number of fruits or vegetables produced per plant at a given time), frequency of production (e.g., the number of flowering cycles per plant in a given season that result in products), and ease of harvest of product (e.g., fruits or vegetables that detach easily from the plant). Examples of quality-related traits include taste, color, shape, firmness, odor, and mouthfeel. Table 1 provides non-limiting list of genes of interest and traits of interest modulated by the gene. More information related to the gene names below may be found, e.g., in the Maize Genetics and Genomics database (maizegdb.org), the Sol Genomics Network database (solgenomics.net), the Arabidoposis database (arabidopsis.org), and the Rice Genome Annotation Project database (rice.plantbiology.msu.edu) database.

TABLE 1
Example Genes of Interest and Traits

| Gene Name | Trait(s) Modulated by Gene | Cited References |
|---|---|---|
| SlCLAVATA1 | Floral organ number, fruit | Xu et al., 2015. Nat. Genet. |
| | size | 47, 784-792. |
| SlCLAVATA2 | Floral organ number, fruit | Xu et al., 2015. Nat. Genet. |
| | size | 47, 784-792. |
| SlCLAVATA3 | Floral organ number, fruit | Xu et al., 2015. Nat. Genet. |
| | size | 47, 784-792. |
| SlWUSCHEL | Floral organ number, fruit | Muños et al., 2011. Plant |
| | size | Physiol. 156, 2244-2254; Li |
| | | et al., 2017. Front. Plant Sci. |
| | | 8, 457. |
| FRUIT WEIGHT 2.2 | Fruit size | Frary et al., 2000. Science. |
| | | 289, 85-88. |
| OVATE | Fruit shape | Liu et al., 2002. PNAS 99, |
| | | 13302-13306. |
| SUN | Fruit shape | Xiao et al., 2008. Science |
| | | 319, 1527-1530. |
| LONG INFLORESCENCE | Fruit number per | Soyk, Lemmon et al., 2017. |
| | inflorescence | Cell, in press. |
| TERMINATING FLOWER | Number of flowers per | MacAlister et al. (2012). Nat. |
| | inflorescence, flowering time | Genet. 44, 1393-8 (2012). |
| SELF PRUNING | Sympodial growth; flowering | Pnueli, L. et al., 1998. |
| | time, plant architecture | Development 125, 1979- |
| | | 1989. |
| SINGLE FLOWER TRUSS | Flowering time, plant | Shalit et al., (2009). PNAS |
| | architecture | 106, 8392-8397. |
| SELF PRUNING 5G | Flowering time, plant | Soyk et al., 2016. Nat. Genet. |
| | architecture | 49, 162-168. |
| COMPOUND | Inflorescence branching | Lippman et al., 2008. PLoS |
| INFLORESCENCE | | Biol. 6, e288. |
| JOINTLESS2 | Fruit abscission, | Soyk, Lemmon et al., 2017. |
| | inflorescence branching | Cell, in press. |
| LIN5 | Sugar levels on fruit | Fridman et al., 2004. Science |
| | | 305: 1786-1789. |
| ENHANCER OF | Calyx size, inflorescence | Soyk, Lemmon et al., 2017. |
| JOINTLESS2 | branching | Cell, in press. |
| SUPPRESSOR OF | Inflorescence architecture | Doebley et al., 1995. Am. J. |
| SESSILE SPIKELETS1 | | Bot. 82, 571-577. |
| BARREN STALK1 | Axillary meristem | Gallavotti et al., 2004. Nature |
| | development | 432, 630-635 |
| ZmCO, CO-LIKE, and | Flowering time | Yang et al., 2013. PNAS 110, |
| TIMING OF CAB1 | | 16969-16974; Ducrocq et al., |
| | | 2009, 183(4):1555-1563. |
| ZmSUGARY1 | Starch biosynthesis, sugary | James et al., 1995. Plant Cell |
| | sweet taste. | 7(4):417-429. |
| BETAINE ALDEHYDE | Fragrant grains | Bradbury et al., 2005. Plant |
| DEHYDROGENASE2 | | Biotech Journal 3:363-370. |
| GRAIN WIDTH5 | Seed size | Weng et al., 2008. Cell Res |
| | | 18:1199-1209. |
| HEADINGDATE1 and 2 | Flowering time | Matsubara et al., 2008. Plant |
| | | Cell 3:1425-1435. |
| FASCIATED EAR2 | Kernel row number, kernel | Bommert, P., Nagasawa, |
| | yield | N.S., Jackson, D. (2013). |
| | | Quantitative Variation in |
| | | Maize Kernel Row Number |
| | | is Controlled by the |
| | | FASCIATED EAR2 Locus. |
| | | Nature Genetics, 45(3): 334- |
| | | 7. |
| FASCIATED EAR3 | Kernel row number, kernel | Je BI, Gruel J, Lee YK, |
| | yield | Bommert P, Arevalo ED, |
| | | Eveland AL, Wu Q, |
| | | Goldshmidt A, Meeley R, |
| | | Bartlett M, Komatsu M, |
| | | Sakai H, Jönsson H, Jackson |
| | | D. (2016). Signaling from |
| | | maize organ primordia via |
| | | FASCIATED EAR3 |
| | | regulates stem cell |
| | | proliferation and yield traits. |
| | | Nat Genet. 2016 May 16. |
| | | doi: 10.1038/ng.3567. |
| FASCIATED EAR4 | Kernel row number, kernel | Pautler, M., Eveland, A., |
| | yield | LaRue, T., Yang, F., Weeks, |
| | | R., Lunde, C., Je, B.I., |
| | | Meeley, R., Komatsu, M., |
| | | Vollbrecht, E., Sakai, H., |
| | | Jackson, D. (2015). |
| | | FASCIATED EAR4 Encodes |
| | | a bZIP Transcription Factor |
| | | that Regulates Shoot |
| | | Meristem Size in Maize. The |
| | | Plant Cell, 27(1): 104-120. |
| ABPHYL1 | phyllotaxy | Giulini, A., Wang, J., |
| | | Jackson, D. (2004). Control |
| | | of Phyllotaxy by the |
| | | Cytokinin Inducible |
| | | Response Regulator |
| | | Homologue ABPHYL1. |
| | | Nature, 430(7003):1031-1034. |
| ABPHYL2 | phyllotaxy | Yang, F., Bui, H.T., Pautler, |
| | | M., Llaca, V., Johnston, R., |
| | | Lee, B.H., Kolbe, A., Sakai, |
| | | H., Jackson, D. (2015). A |
| | | Maize Glutaredoxin Gene, |
| | | Abphyl2, Regulates Shoot |
| | | Meristem Size and |
| | | Phyllotaxy. The Plant Cell, |
| | | 27(1): 121-131. |
| RAMOSA3 | Kernel row number, kernel | Satoh-Nagasawa, N. |
| | yield, branching | Nagasawa, N., Malcomber, |
| | | S., Sakai, H., Jackson, D. |
| | | (2006). A Trehalose |
| | | Metabolic Enzyme Controls |
| | | Inflorescence Architecture in |
| | | Maize. Nature, 441(7090): |
| | | 227-230. |
| COMPACT PLANT2 | Kernel row number, kernel | Bommert, P., Je, B., |
| | yield | Goldshmidt, A., Jackson, D. |
| | | (2013). The Maize Gα Gene |
| | | COMPACT PLANT2 |
| | | Functions in CLAVATA |
| | | Signalling to Control Shoot |
| | | Meristem Size. Nature, |
| | | 502(7472): 555-558. |
| ZmCLE7 | Kernel row number, kernel | Je BI, Gruel J, Lee YK, |
| | yield | Bommert P, Arevalo ED, |
| | | Eveland AL, Wu Q, |
| | | Goldshmidt A, Meeley R, |
| | | Bartlett M, Komatsu M, |
| | | Sakai H, Jönsson H, Jackson |
| | | D. (2016). Signaling from |
| | | maize organ primordia via |
| | | FASCIATED EAR3 |
| | | regulates stem cell |
| | | proliferation and yield traits. |
| | | Nat Genet. 2016 May 16. |
| | | doi: 10.1038/ng.3567. |
| ZmFCP1 | Kernel row number, kernel | Je BI, Gruel J, Lee YK, |
| | yield | Bommert P, Arevalo ED, |
| | | Eveland AL, Wu Q, |
| | | Goldshmidt A, Meeley R, |
| | | Bartlett M, Komatsu M, |
| | | Sakai H, Jönsson H, Jackson |
| | | D. (2016). Signaling from |
| | | maize organ primordia via |
| | | FASCIATED EAR3 |
| | | regulates stem cell |
| | | proliferation and yield traits. |
| | | Nat Genet. 2016 May 16. |
| | | doi: 10.1038/ng.3567. |

Other example genes of interest and traits of interest are described, e.g., in Meyer et al. Evolution of crop species: genetics of domestication and diversification. Nat. Rev. Genet. 14, 840-52 (2013); Olsen et al. A bountiful harvest: genomic insights into crop domestication phenotypes. Annu. Rev. Plant Biol. 64, 47-70 (2013); Zhang et al. Molecular Control of Grass Inflorescence Development. Annu. Rev. Plant Biol. 65, 553-578 (2014); Park et al. Meristem maturation and inflorescence architecture—lessons from the Solanaceae. Curr. Opin. Plant Biol. 17, 70-77 (2014); and Kyozuka et al. Control of grass inflores-cence form by the fine-tuning of meristem phase change. Curr. Opin. Plant Biol. 17, 110-115 (2014).

In some embodiments, the library contains a plurality of crop plants or a plurality of seeds of crop plants. Crop plants include any plant that produces grain, nuts, legumes, seeds, roots, tubers, leaves, vegetables or fruit that are edible or otherwise usable (such as in medicine or recreationally) by mammals, such as humans or livestock, or that produces fibers useful for manufacturing textiles. Crop plants include, for example, Solanaceae plants (e.g., tomato, potato, eggplant, tobacco, and pepper), cotton, cassava, rapeseed, canola, barley, oats, maize, sorghum, soybeans, legumes, wheat and rice. In some embodiments, each member of the library is of the same type of plant (e.g., the same type of crop plant, such as each member is a tomato plant or maize plant).

In some embodiments, each plant or seed in the plurality is an F1 hybrid plant or seed. As used herein, an “F1 hybrid” means that the plant or seed was generated by crossing together two different parent plants that have different genotypes for at least one location in the genome. For example, one parent plant may contain an expression cassette as described herein (e.g., a CRISPR/RNA-guided endonuclease expression cassette such as a CRISPR/Cas9 expression cassette or a CRISPR/Cpf1 expression cassette as described herein) and the other parent plant may contain a first allele as described herein such that the F1 hybrid plant or seed generated by crossing the parent plants may contain both the expression cassette and the first allele.

In some embodiments, the library contains at least 50 (e.g., at least 50, at least 100, at least 500, or at least 5000) members. In some embodiments, the library contains between 10 and 10000 members (e.g., between 10 and 10000, 10 and 5000, 10 and 1000, 10 and 500, 10 and 100, 10 and 50, 50 and 10000, 50 and 5000, 50 and 1000, 50 and 500, 50 and 100, 100 and 10000, 100 and 5000, 100 and 1000, 100 and 500, 500 and 10000, 500 and 5000, or 500 and 1000 members). In some embodiments, the plurality of plants or seeds that each contain an expression cassette as described herein (e.g., a CRISPR/RNA-guided endonuclease expression cassette such as a CRISPR/Cas9 expression cassette or a CRISPR/Cpf1 expression cassette as described herein) makes up at least 10%, at least 20%, at least 30%, at least 40%, or at least 50% of the library. In some embodiments, the other members of the library that are not in the plurality are plants or seeds that do not contain the expression cassette (e.g., if the parent plant(s) that create the library are hemizygous for the CRISPR/RNA-guided endonuclease expression cassette such as a CRISPR/Cas9 expression cassette or a CRISPR/Cpf1 expression cassette, then not every member of the library will receive a copy of the CRISPR/RNA-guided endonuclease expression cassette). In some embodiments, the plurality contains at least 50 (e.g., at least 50, at least 100, at least 500, or at least 5000) members. In some embodiments, the plurality contains between 10 and 10000 members (e.g., between 10 and 10000, 10 and 5000, 10 and 1000, 10 and 500, 10 and 100, 10 and 50, 50 and 10000, 50 and 5000, 50 and 1000, 50 and 500, 50 and 100, 100 and 10000, 100 and 5000, 100 and 1000, 100 and 500, 500 and 10000, 500 and 5000, or 500 and 1000 members).

In some embodiments, each plant or seed in the plurality contains a first allele and a second allele of a gene of interest. In some embodiments, the first allele contains a mutation in a regulatory region of the gene of interest, a coding region of the gene of interest or both (e.g., a missense mutation, a nonsense mutation, an insertion, a deletion, a duplication, an inversion, or a translocation, or a combination of structural variations thereof such as an indel, e.g., containing both an insertion of nucleotides and a deletion of nucleotides which may result in a net change in the total number of nucleotides). In some embodiments, the regulatory region is a promoter. In some embodiments, the mutation in the coding region is in an exon. In some embodiments, the first allele is a hypomorphic allele or a null allele. In some embodiments, a hypomorphic allele is an allele that results in an mRNA or protein expression level of the gene of interest that is at least 20% lower (e.g., at least 20%, at least 30%, at least 40%, at least 50%, at least 60%, at least 70%, at least 80% or at least 90%) than an allele of the gene of interest that does not contain the mutation (e.g., a wild-type allele). As used herein, a “null allele” refers to an allele of a gene of interest in which transcription into RNA does not occur, translation into a functional protein does not occur or neither occurs due to a mutation which may be located within the coding sequence, in a regulatory region of the gene, or both (e.g., a missense mutation, a nonsense mutation, an insertion, a deletion, a duplication, an inversion, or a translocation, or a combination of structural variations thereof such as an indel). In some embodiments, the null allele is a knock-out allele. As used herein, a “knock-out allele” refers to an allele of a gene in which transcription into RNA does not occur, translation into a functional protein does not occur or neither occurs as a result of a deletion of some portion or all of the coding sequence of the gene, e.g., using homologous recombination. One non-limiting approach to creating knock-out mutations is to use CRISPR/RNA-guided endonuclease mutagenesis (e.g., CRISPR/Cas9 mutagenesis or CRISPR/Cpf1 mutagenesis) to target exons that encode functional protein domains or to target a large portion (e.g., at least 80%) or the entirety of the coding sequence (see, e.g., Shi et al. Nature Biotechnology. (2015) 33(6): 661-667 and Online Methods). Other mutagenesis techniques may also be used to produce a hypomorphic or null first allele, for example, by introducing mutations in the first allele through transposon insertions, EMS mutagenesis, fast neutron mutagenesis, or other applicable mutagenesis methods. In some embodiments, a hypomorphic or null first allele may be produced using a method as described herein for producing gRNA/endonuclease-induced mutations (e.g., using a CRISPR/RNA-guided endonuclease expression construct (e.g., a CRISPR/Cas9 expression construct or a CRISPR/Cpf1 expression construct) as described herein to induce gRNA/RNA-guided endonuclease mutations (such as Cas9 mutations or Cpf1 mutations) and selecting a mutated first allele that is a hypomorphic or null allele).

In some embodiments, the second allele that contains the target region against which the multiple guide RNAS (gRNAs), such as single-guide RNAs (sgRNAs), are designed is a naturally-occurring allele (e.g., an allele naturally present in a plant, such as a crop plant). In some embodiments, the second allele is not a hypomorphic allele or a null allele. In some embodiments, the expression cassette (e.g., the CRISPR/RNA-guided endonuclease expression cassette such as a CRISPR/Cas9 expression cassette or a CRISPR/Cpf1 expression cassette) is active in at least one member of the plurality such that at least one gRNA/endonuclease-induced mutation (e.g., at least one gRNA/Cas9-induced mutation or at least one gRNA/Cpf1-induced mutation) occurs in the second allele. In some embodiments, at least 10%, at least 20%, at least 30%, at least 40%, at least 50% or more of the members of the plurality contain at least one gRNA/endonuclease-induced mutation (e.g., at least one gRNA/Cas9-induced mutation or at least one gRNA/Cpf1-induced mutation) in the second allele. In some embodiments, the gRNA/RNA-guided endonuclease-induced mutation (e.g., a Cas9-induced mutation or a Cp1-inducted mutation) is a deletion, insertion, inversion, or translocation, or a combination of structural variations thereof, such as an indel. It is to be understood that the gRNA/endonuclease-induced mutation (e.g., gRNA/Cas9-induced mutation or gRNA/Cpf1-induced mutation) does not have to be the same in each member and generally will not be the same in each member, especially if 4 or more gRNAs (e.g., sgRNAs) are present in the expression cassette (e.g., CRISPR/RNA-guided endonuclease expression cassette such as a CRISPR/Cas9 expression cassette or a CRISPR/Cpf1 expression cassette). In some embodiments, the expression cassette (e.g., CRISPR/RNA-guided endonuclease expression cassette such as a CRISPR/Cas9 expression cassette or a CRISPR/Cpf1 expression cassette) is not active in the members of the plurality, e.g., if the library members are dormant seeds that have not undergone germination such that the expression cassette (e.g., CRISPR/RNA-guided endonuclease expression cassette such as a CRISPR/Cas9 expression cassette or a CRISPR/Cpf1 expression cassette) is not actively transcribed. In some embodiments, the expression cassette (e.g., CRISPR/RNA-guided endonuclease expression cassette such as a CRISPR/Cas9 expression cassette or a CRISPR/Cpf1 expression cassette) is active or has been active in at least some of the members of the plurality, e.g., if the library members are seeds undergoing development (e.g., embryogenesis) or germination or if the library members are plants, such that the expression cassette (e.g., CRISPR/RNA-guided endonuclease expression cassette such as a CRISPR/Cas9 expression cassette or a CRISPR/Cpf1 expression cassette) is or has been actively transcribed.

### Methods

In other aspects, the disclosure provides methods of generating libraries. In some embodiments, the libraries generated contain a plurality of plants or seeds as described herein.

In some embodiments, the method comprises (a) providing a first plant comprising a gene of interest comprising a coding sequence and (i) having a first allele of the gene of interest (e.g., that is a hypomorphic allele or a null allele as described herein) and (ii) an expression cassette (e.g., CRISPR/RNA-guided endonuclease expression cassette such as a CRISPR/Cas9 expression cassette or a CRISPR/Cpf1 expression cassette) as described herein (e.g., that encodes a Cas9, a Cpf1, or a Csm1 endonuclease as described herein and at least 2 (e.g., at least 2, at least 3, at least 4, at least 5, at least 6, at least 7, at least 8 or at least 9, such as 4 to 8 or 4 to 9) different gRNAs, e.g., sgRNAs, as described herein); (b) providing a second plant comprising (i) a second allele of the gene of interest that is different from the first allele (e.g., that is a naturally-occurring allele as described herein or is not a hypomorphic allele or a null allele as described herein); and (c) crossing the first plant to the second plant to produce a plurality of plants or seeds (e.g., F1 hybrid plants or seeds), each plant or seed in the plurality comprising the first allele, the second allele and the expression cassette (e.g., CRISPR/RNA-guided endonuclease expression cassette such as a CRISPR/Cas9 expression cassette or a CRISPR/Cpf1 expression cassette). In some embodiments, the first plant is hemizygous for the expression cassette (e.g., CRISPR/RNA-guided endonuclease expression cassette). In some embodiments, the first plant is homozygous for the expression cassette (e.g., CRISPR/RNA-guided endonuclease expression cassette such as a CRISPR/Cas9 expression cassette or a CRISPR/Cpf1 expression cassette). In some embodiments, the first plant is homozygous for the first allele and the second plant is homozygous for the second allele. In some embodiments, the first plant is heterozygous for the first allele and the second plant is homozygous for the second allele. In some embodiments, the first plant is homozygous for the first allele and the second plant is heterozygous for the second allele. In some embodiments, the first plant is heterozygous for the first allele and the second plant is heterozygous for the second allele. In some embodiments, the first plant is hemizygous for the expression cassette (e.g., CRISPR/RNA-guided endonuclease expression cassette such as a CRISPR/Cas9 expression cassette or a CRISPR/Cpf1 expression cassette) and homozygous for the first allele.

In some embodiments, the method comprises (a) providing a first plant comprising a gene of interest comprising a coding sequence and having a first allele of the gene of interest (e.g., that is a hypomorphic allele or a null allele as described herein), (b) providing a second plant comprising (i) a second allele of the gene of interest that is different from the first allele (e.g., that is a naturally-occurring allele as described herein or is not a hypomorphic allele or a null allele as described herein), and (ii) an expression cassette (e.g., CRISPR/RNA-guided endonuclease expression cassette such as a CRISPR/Cas9 expression cassette or a CRISPR/Cpf1 expression cassette) as described herein (e.g., that encodes a Cas9, a Cpf1, or a Csm1 endonuclease as described herein and at least 2 (e.g., at least 2, at least 3, at least 4, at least 5, at least 6, at least 7, at least 8 or at least 9, such as 4 to 8 or 4 to 9) different gRNAs, e.g., sgRNAs, as described herein) and (c) crossing the first plant to the second plant to produce a plurality of plants or seeds (e.g., F1 hybrid plants or seeds), each plant or seed in the plurality comprising the first allele, the second allele and the expression cassette (e.g., CRISPR/RNA-guided endonuclease expression cassette such as a CRISPR/Cas9 expression cassette or a CRISPR/Cpf1 expression cassette). In some embodiments, the second plant is hemizygous for the expression cassette (e.g., CRISPR/RNA-guided endonuclease expression cassette such as a CRISPR/Cas9 expression cassette or a CRISPR/Cpf1 expression cassette). In some embodiments, the second plant is homozygous for the expression cassette (e.g., CRISPR/RNA-guided endonuclease expression cassette such as a CRISPR/Cas9 expression cassette or a CRISPR/Cpf1 expression cassette). In some embodiments, the first plant is homozygous for the first allele and the second plant is homozygous for the second allele. In some embodiments, the first plant is heterozygous for the first allele and the second plant is homozygous for the second allele. In some embodiments, the first plant is homozygous for the first allele and the second plant is heterozygous for the second allele. In some embodiments, the first plant is heterozygous for the first allele and the second plant is heterozygous for the second allele. In some embodiments, the second plant is hemizygous for the expression cassette (e.g., CRISPR/RNA-guided endonuclease expression cassette such as a CRISPR/Cas9 expression cassette or a CRISPR/Cpf1 expression cassette) and homozygous for the second allele.

In some embodiments of any of the methods provided herein, the method further comprises maintaining the plurality of plants or seeds (e.g., F1 hybrid plants or F1 hybrid seeds) under conditions in which the gRNA/endonuclease (e.g., gRNA/Cas9) induces mutations within the target region of the second allele. In some embodiments, a constitutive promoter (e.g., a CaMV 35s promoter, a maize U6 promoter, a rice U6 promoter, or a maize Ubiquitin promoter) is used to drive expression of the expression cassette (e.g., CRISPR/RNA-guided endonuclease expression cassette such as a CRISPR/Cas9 expression cassette or a CRISPR/Cpf1 expression cassette) such that the conditions in which the mutations are induced are conditions that permit growth of the plants or germination of the seeds. Conditions for permitting growth and germination of seeds of various plants, such as crop plants, are known in the art and are described herein with respect to tomatoes as an example crop plant. In some embodiments, an inducible promoter is used to drive expression of the expression cassette (e.g., CRISPR/RNA-guided endonuclease expression cassette such as a CRISPR/Cas9 expression cassette or a CRISPR/Cpf1 expression cassette) and the conditions in which the mutations are induced are conditions under which the inducible promoter is active, e.g., upon addition of ethanol, dexamethasone, or beta-estradiol or upon exposure to a change in temperature (e.g., heat shock).

Other aspects of the disclosure relate to methods of selecting members of a library having a phenotype of interest. In some embodiments, the phenotype of interest is a yield-related trait or quality-related trait as described herein, e.g., a trait in Table 1. In some embodiments, the method comprises (a) providing a plant library or seed library as described herein (e.g., comprising a plurality of plants or seeds such as F1 hybrid plants or F1 hybrid seeds as described herein); (b) selecting at least one member of the library that exhibits a phenotype of interest; and (c) crossing the at least one member to at least one other plant (a plant that does not contain the expression cassette, e.g., CRISPR/RNA-guided endonuclease expression cassette such as a CRISPR/Cas9 expression cassette or a CRISPR/Cpf1 expression cassette as described herein). In some embodiments, the other plant comprises a null allele of a gene of interest (e.g., a null allele). In some embodiments, the other plant comprises a mutation in a second gene, such as a gene that affects the same phenotype as the phenotype affected by the gene of interest (e.g., is part of the same pathway or has some level of redundancy with the gene of interest). Yet other aspects of the disclosure relate to plant libraries, seed libraries, plants, seeds, plant cells, and isolated DNA obtainable by any of the methods described herein.

### Nucleic Acids

In yet other aspects, the disclosure provides nucleic acids comprising an expression cassette (e.g., CRISPR/RNA-guided endonuclease expression cassette such as a CRISPR/Cas9 expression cassette or a CRISPR/Cpf1 expression cassette) as described herein. In some embodiments, the expression cassette (e.g., CRISPR/RNA-guided endonuclease expression cassette such as a CRISPR/Cas9 expression cassette or a CRISPR/Cpf1 expression cassette) encodes a RNA-guided endonuclease (e.g., a Cas9, a Cpf1, or a Csm1 endonuclease) and at least two (e.g., at least 2, at least 3, at least 4, at least 5, at least 6, at least 7, at least 8 or at least 9, such as 4 to 8 or 4 to 9) different gRNAs (e.g., sgRNAs), each gRNA containing a sequence that is complementary to a target sequence within a target region in a gene of interest. In some embodiments, the cassette contains between two and sixteen (e.g., 2, 3, 4, 5, 6, 7, 8, 9, 10, 11, 12, 13, 14, 15, or 16) different gRNAs (e.g., sgRNAs). In some embodiments, each target sequence in the target region is located 50 to 500 base pairs (e.g., 50 to 500, 50 to 400, 50 to 300, 50 to 200, 50 to 100, 100 to 500, 100 to 400, 100 to 300, 100 to 200, 200 to 500, 200 to 400, or 200 to 300) away from at least one other different target sequence. In some embodiments, each target sequence is located next to a Protospacer Adjacent Motif (PAM) sequence, such as NGG, NAA, NNNNGATT, NNAGAA, or NAAAAC. In some embodiments, the PAM sequence is a Cpf1 or Csm1 PAM sequence, such as TTN, CTA, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, or CCGN. In some embodiments, each gRNA is a single-guide RNA (sgRNA) containing a trans-activating CRISPR RNA (tracrRNA) and a CRISPR RNA (crRNA) designed to cleave the target site of interest. In some embodiments, the gRNA is a sgRNA containing a crRNA. In some embodiments, the RNA-guided endonuclease is a Cas9 endonuclease or a Cpf1 endonuclease or a Csm1 endonuclease, or a functional variant thereof.

In some embodiments, the RNA-guided endonuclease is a Cas9 endonuclease. The Cas9 endonuclease may be any Cas9 endonuclease known in the art or described herein. In some embodiments, the Cas9 endonuclease is a rice optimized CAS9 (see, e.g., Jiang et al. Demonstration of CRISPR/Cas9/sgRNA-mediated targeted gene modification in Arabidopsis, tobacco, sorghum and rice, Nucleic Acids Res. 2013 November; 41(20):e188). In some embodiments, the Cas9 endonuclease has an amino acid sequence that is at least 90%, 95%, 98%, 99% or 100% identical to the following amino acid sequence:

(SEQ ID NO: 1)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGDSRAD
PKKKRKV.

In some embodiments, the RNA-guided endonuclease is a Cpf1 endonuclease. The Cpf1 endonuclease may be any Cpf1 endonuclease known in the art or described herein (e.g., FnCpf1, AsCpf1, Lb2Cpf1, CMtCpf1, MbCpf1, LbCpf1, PcCpf1, or PdCpf1, see, e.g., U.S. Pat. No. 9,896,696). In some embodiments, the RNA-guided endonuclease is a Csm1 endonuclease. The Csm1 endonuclease may be any Csm1 endonuclease known in the art or described herein (e.g., SsCsm1, SmCsm1, ObCsm1, Sm2Csm1, or MbCsm1, see, e.g., U.S. Pat. No. 9,896,696

In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest (e.g., the second allele of the gene of interest). In some embodiments, the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest (e.g., the second allele of the gene of interest). In some embodiments, if the crop is a cereal crop (such as maize), the target region may be 0 to 100 kilobases (e.g., 0 to 100, 0 to 90, 0 to 80, 0 to 70, 0 to 60, 0 to 50, 0 to 40, 0 to 30, 0 to 20 or 0 to 10 kilobases) upstream of the 5′ end of the coding sequence of the gene of interest (e.g., the second allele of the gene of interest). In some embodiments, the target region is 0 to 60 kilobases (e.g., 0 to 60, 0 to 50, 0 to 40, 0 to 30, 0 to 20 or 0 to 10 kilobases) base pairs downstream of the 3′ end of the coding sequence of the gene of interest (e.g., the second allele of the gene of interest).

In some embodiments, the target region comprises a regulatory region of the gene of interest. In some embodiments, the regulatory region comprises a transcription factor binding site, an RNA polymerase binding site, a TATA box, or a combination thereof. In some embodiments, the regulatory region is within a certain distance of the gene of interest, e.g., 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest.

In some embodiments, the expression cassette (e.g., CRISPR/RNA-guided endonuclease expression cassette such as a CRISPR/Cas9 expression cassette or a CRISPR/Cpf1 expression cassette) contains a constitutive promoter, e.g., a CaMV 35s promoter. a maize U6 promoter, a rice U6 promoter, or a maize Ubiquitin promoter. In some embodiments, the expression cassette (e.g., CRISPR/RNA-guided endonuclease expression cassette such as a CRISPR/Cas9 expression cassette or a CRISPR/Cpf1 expression cassette) contains a tissue-specific promoter, such as an anther-specific promoter or a pollen-specific promoter. In some embodiments, the expression cassette (e.g., CRISPR/RNA-guided endonuclease expression cassette such as a CRISPR/Cas9 expression cassette or a CRISPR/Cpf1 expression cassette) contains an inducible promoter, such as an ethanol inducible promoter, a dexamethasone inducible promoter, a beta-estradioal inducible promoter, or a heat shock inducible promoter. In some embodiments, the same promoter is used to drive expression of both the RNA-guided endonuclease (e.g., Cas9, Cpf1, or Csm1) sequence and the gRNA sequences. In some embodiments, different promoters are used to drive the expression of the RNA-guided endonuclease (e.g., Cas9, Cpf1, or Csm1) sequence and the gRNA sequences. In some embodiments, expression of the gRNAs is driven a using a polycistronic tRNA system.

In some embodiments, the nucleic acid is a vector, such as a plasmid. In some embodiments, a suitable vector, such as a plasmid, contains an origin of replication functional in at least one organism, convenient restriction endonuclease or other cloning sites, and one or more selectable markers. In some embodiments, the nucleic acid is contained within a cell. In some embodiments, the cell is plant cell (e.g., a crop plant cell). In some embodiments, the plant cell is isolated. In some embodiments, the plant cell is a non-replicating plant cell. In some embodiments, the cell is a bacterial cell (e.g., E. coli or Agrobacterium tumefaciens).

### Further Embodiments

The following are further non-limiting embodiments of the disclosure.

Clause 1. A plant library comprising a plurality of F1 hybrid plants, each F1 hybrid plant in the plurality comprising:

(a) a gene of interest comprising a coding sequence and having a first allele that is a hypomorphic allele or a null allele and a second allele that is different from the first allele, and

(b) an expression cassette that encodes a RNA-guided endonuclease and at least four different guide RNAs (gRNAs), each gRNA containing a sequence that is complementary to a target sequence within a target region in the second allele of the gene of interest,

wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest.

Clause 2. A seed library comprising a plurality of F1 hybrid seeds, each F1 hybrid seed in the plurality comprising:

(a) a gene of interest comprising a coding sequence and having a first allele that is a hypomorphic allele or a null allele and a second allele that is different from the first allele, and

(b) an expression cassette that encodes a RNA-guided endonuclease and at least four different guide RNAs (gRNAs), each gRNA containing a sequence that is complementary to a target sequence within a target region in the second allele of the gene of interest,

wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest.

Clause 3. The library of clause 1 or 2, wherein the target region comprises a regulatory region of the gene of interest.
Clause 4. The library of clause 3, wherein the regulatory region comprises a transcription factor binding site, an RNA polymerase binding site, a TATA box, or a combination of structural variations thereof.
Clause 5. The library of clause 3 or 4, wherein the regulatory region is a promoter.
Clause 6. The library of any one of clauses 1 to 5, wherein the expression cassette encodes at least five different gRNAs.
Clause 7. The library of clause 6, wherein the expression cassette encodes at least six different gRNAs.
Clause 8. The library of clause 6, wherein the expression cassette encodes at least seven different gRNAs.
Clause 9. The library of clause 6, wherein the expression cassette encodes at least eight different gRNAs.
Clause 10. The library of clause 6, wherein the expression cassette encodes four to nine (e.g., 4, 5, 6, 7, 8 or 9) different gRNAs.
Clause 11. The library of clause 6, wherein the expression cassette encodes five to eight different gRNAs.
Clause 12. The library of any one of clauses 1 to 5, wherein the expression cassette encodes six to eight different gRNAs.
Clause 13. The library of any one of clauses 1 to 12, wherein the second allele is a naturally-occurring allele.
Clause 14. The library of any one of clauses 1 to 13, wherein the second allele is not a hypomorphic allele.
Clause 15. The library of any one of clauses 1 to 13, wherein the second allele is not a null allele.
Clause 16. The library of any one of clauses 1 to 15, wherein the first allele contains a mutation in a regulatory region of the gene of interest.
Clause 17. The library of any one of clauses 1 to 15, wherein the first allele contains a mutation in a coding sequence of the gene of interest.
Clause 18. The library of clause 16 or 17, wherein the first allele is a hypomorphic allele that results in an mRNA expression level of the gene of interest that is at least 70% lower than an allele of the gene of interest that does not contain the mutation.
Clause 19. The library of any one of clauses 1 to 18, wherein the RNA-guided endonuclease is a Cas9 endonuclease (e.g., having an amino acid sequence that is at least 90%, 95%, 98%, 99% or 100% identical to SEQ ID NO: 1), optionally wherein each gRNA is a single-guide RNA (sgRNA).
Clause 19A. The library of any one of clauses 1 to 18, wherein the RNA-guided endonuclease is a Cpf1 endonuclease, optionally wherein each gRNA is a single-guide RNA (sgRNA).
Clause 19B. The library of any one of clauses 1 to 18, wherein the RNA-guided endonuclease is a Csm1 endonuclease, optionally wherein each gRNA is a single-guide RNA (sgRNA).
Clause 20. The library of any one of clauses 1 to 19B, wherein each target sequence is located 50 to 500 base pairs (e.g., 50 to 500, 50 to 400, 50 to 300, 50 to 200, 50 to 100, 100 to 500, 100 to 400, 100 to 300, 100 to 200, 200 to 500, 200 to 400, or 200 to 300 base pairs) away from at least one other target sequence.
Clause 21. The library of any one of clauses 1 to 20, wherein the library contains at least 50 members (e.g., at least 50, at least 100, at least 500, or at least 5000 members) or contains between 10 and 10000 members (e.g., between 10 and 10000, 10 and 5000, 10 and 1000, 10 and 500, 10 and 100, 10 and 50, 50 and 10000, 50 and 5000, 50 and 1000, 50 and 500, 50 and 100, 100 and 10000, 100 and 5000, 100 and 1000, 100 and 500, 500 and 10000, 500 and 5000, or 500 and 1000 members).
Clause 22. The library of any one of clauses 1 to 21, wherein the plant or seed is a crop plant or crop seed (e.g., a tomato or maize plant or a tomato or maize seed).
Clause 23. The library of any one of clauses 1 to 22, wherein the library is a plant library and at least one member (e.g., at least 10%, at least 20%, at least 30%, at least 40%, at least 50% or more) of the library contains a gRNA/endonuclease-induced (e.g., gRNA/Cas9-induced) mutation in the second allele.
Clause 24. The library of clause 23, wherein the gRNA/endonuclease-induced (e.g., gRNA/Cas9-induced mutation) is a deletion, inversion, translocation or insertion, or a combination of structural variations thereof, such as an indel.
Clause 25. A method of generating a plant library comprising a plurality of F1 hybrid plants, the method comprising:
(a) providing a first plant comprising
   (i) a gene of interest comprising a coding sequence and having a first allele that is a hypomorphic allele or a null allele, and
   (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the expression cassette.
   Clause 26. A method of generating a seed library comprising a plurality of F1 hybrid seeds, the method comprising:
(a) providing a first plant comprising
   (i) a gene of interest comprising a coding sequence and having a first allele that is a hypomorphic allele or a null allele, and
   (ii) an expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the expression cassette.
   Clause 27. The method of clause 25 or 26, wherein the first plant is hemizygous for the expression cassette.
   Clause 28. The method of any one of clauses 25 to 27, wherein the first plant is homozygous for the first allele and the second plant is homozygous for the second allele.
   Clause 29. The method of any one of clauses 25 to 28, wherein the method further comprises maintaining the plurality of F1 hybrid plants or F1 hybrid seeds under conditions that permit the gRNA/endonuclease to induce mutations within the target region of the second allele.
   Clause 30. The method of any one of clauses 25 to 29, wherein the RNA-guided endonuclease is a Cas9 endonuclease (e.g., having an amino acid sequence that is at least 90%, 95%, 98%, 99% or 100% identical to SEQ ID NO: 1), optionally wherein each gRNA is a single-guide RNA (sgRNA).
   Clause 30A. The method of any one of clauses 25 to 29, wherein the RNA-guided endonuclease is a Cpf1 endonuclease, optionally wherein each gRNA is a single-guide RNA (sgRNA).
   Clause 30B. The method of any one of clauses 25 to 29, wherein the RNA-guided endonuclease is a Csm1 endonuclease, optionally wherein each gRNA is a single-guide RNA (sgRNA).
   Clause 31. A method of selecting members of a library having a phenotype of interest, the method comprising:
(a) providing a plant or seed library of any one of clauses 1 to 24,
(b) selecting at least one member of the library that exhibits a phenotype of interest, and
(c) crossing the at least one member to at least one plant that does not contain the expression cassette.
   Clause 31A. A plant obtainable or obtained by the method of clause 31.
   Clause 32. A plant library comprising a plurality of F1 hybrid plants obtainable, or obtained by, a process comprising:
(a) providing a first plant comprising
   (i) a gene of interest comprising a coding sequence and having a first allele that is a hypomorphic allele or a null allele, and
   (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the expression cassette.
   Clause 33. A seed library comprising a plurality of F1 hybrid seeds obtainable, or obtained by, a process comprising:
(a) providing a first plant comprising
   (i) a gene of interest comprising a coding sequence and having a first allele that is a hypomorphic allele or a null allele, and
   (ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the expression cassette.
   Clause 34. The plant or seed library of clauses 32 or 33, wherein the first plant is hemizygous for the expression cassette.
   Clause 35. The plant or seed library of any one of clauses 32 to 34, wherein the first plant is homozygous for the first allele and the second plant is homozygous for the second allele.
   Clause 36. The plant or seed library of any one of clauses 32 to 35, wherein the method further comprises maintaining the plurality of F1 hybrid plants or F1 hybrid seeds under conditions that permit the gRNA/Cas9 to induce mutations within the target region of the second allele.
   Clause 37. The plant or seed library of any one of clauses 32 to 36, wherein the RNA-guided endonuclease is a Cas9 endonuclease (e.g., having an amino acid sequence that is at least 90%, 95%, 98%, 99% or 100% identical to SEQ ID NO: 1), optionally wherein each gRNA is a single-guide RNA (sgRNA).
   Clause 37-1. The plant or seed library of any one of clauses 32 to 36, wherein the RNA-guided endonuclease is a Cpf1 endonuclease, optionally wherein each gRNA is a single-guide RNA (sgRNA).
   Clause 37-2. The plant or seed library of any one of clauses 32 to 36, wherein the RNA-guided endonuclease is a Csm1 endonuclease, optionally wherein each gRNA is a single-guide RNA (sgRNA).
   Clause 37A. A plant or seed (e.g., a crop plant or crop seed, such as a tomato plant or seed or a maize plant or seed) that is homozygous for a second allele of a gene of interest containing at least one gRNA/Cas9-induced mutation obtainable, or obtained by, a process comprising:
(a) providing a first plant comprising
   (i) a gene of interest comprising a coding sequence and having a first allele that is a hypomorphic allele or a null allele, and
   (ii) an expression cassette that encodes a RNA-guided endonuclease and at least four different guide RNAs (gRNAs, e.g., 4, 5, 6, 7, 8 or 9 different gRNAs), each gRNA containing a sequence that is complementary to a target sequence within a target region in a second allele of the gene of interest that is different from the first allele, wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest,
(b) providing a second plant comprising the second allele of the gene of interest,
(c) crossing the first plant to the second plant to produce a plurality of F1 hybrid plants, each F1 hybrid plant in the plurality comprising the first allele, the second allele and the CRISPR/Cas9 expression cassette,
(d) maintaining the plurality of F1 hybrid plants under conditions that permit the gRNA/Cas9 to induce mutations within the target region of the second allele,
(e) selecting an F1 hybrid plant of step (d) having a phenotype of interest, and
(f) performing a cross (e.g., a self-cross or an outcross such as at least two outcrosses) with the F1 hybrid plant to produce a progeny plant or seed that is homozygous for the second allele containing at least one gRNA/Cas9-induced mutation.
   Clause 37B. The plant or seed of clause 37A, wherein the mutation is a deletion, inversion, translocation or insertion, or a combination of structural variations thereof, such as an indel.
   Clause 37C. A plant cell or seed cell obtainable, or obtained by, a process comprising isolating a cell from the plant or seed of clause 37A or 37B.
   Clause 37D. An isolated DNA molecule comprising a second allele of a gene of interest containing at least one gRNA/Cas9-induced mutation or a fragment of the second allele containing the target region containing the at least one gRNA/Cas9-induced mutation, the DNA molecule obtainable, or obtained by, a process comprising isolating a DNA molecule comprising the second allele, or the fragment thereof, from the plant or seed of clause 37A or 37B or from the plant cell or seed cell of clause 37C.
   Clause 38. A nucleic acid comprising an expression construct encoding a RNA-guided endonuclease and at least four different guide RNAs (gRNAs), each gRNA containing a sequence that is complementary to a target sequence within a target region in an allele of a gene of interest in a plant, wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) upstream of the 5′ end of the coding sequence of the gene of interest or wherein the target region is 0 to 5000 base pairs (e.g., 0 to 5000, 0 to 4000, 0 to 3000, 0 to 2000, 0 to 1000, 100 to 5000, 100 to 4000, 100 to 3000, 100 to 2000, 100 to 1000, 500 to 5000, 500 to 4000, 500 to 3000, 500 to 2000, 500 to 1000, 1000 to 5000, 1000 to 4000, 1000 to 3000, or 1000 to 2000 base pairs) downstream of the 3′ end of the coding sequence of the gene of interest.
   Clause 39. The nucleic acid of clause 38, wherein the target region comprises a regulatory region of the gene of interest.
   Clause 40. The nucleic acid of clause 40, wherein the regulatory region comprises a transcription factor binding site, an RNA polymerase binding site, a TATA box, or a combination thereof
   Clause 41. The nucleic acid of clause 39 or 40, wherein the regulatory region is a promoter.
   Clause 42. The nucleic acid of any one of clauses 38 to 41, wherein the expression cassette encodes at least five different gRNAs.
   Clause 43. The nucleic acid of clause 42, wherein the expression cassette encodes at least six different gRNAs.
   Clause 44. The nucleic acid of clause 42, wherein the expression cassette encodes at least seven different gRNAs.
   Clause 45. The nucleic acid of clause 42, wherein the expression cassette encodes at least eight different gRNAs.
   Clause 46. The nucleic acid of any one of clauses 38 to 41, wherein the expression cassette encodes four to nine (e.g., 4, 5, 6, 7, 8 or 9) different gRNAs.
   Clause 47. The nucleic acid of clause 46, wherein the expression cassette encodes five to eight different gRNAs.
   Clause 48. The nucleic acid of clause 46, wherein the expression cassette encodes six to eight different gRNAs.
   Clause 49. The nucleic acid of any one of clauses 38 to 48, wherein the RNA-guided endonuclease is a Cas9 endonuclease (e.g., having an amino acid sequence that is at least 90%, 95%, 98%, 99% or 100% identical to SEQ ID NO: 1), optionally wherein each gRNA is a single-guide RNA (sgRNA).
   Clause 49A. The nucleic acid of any one of clauses 38 to 48, wherein the RNA-guided endonuclease is a Cpf1 endonuclease, optionally wherein each gRNA is a single-guide RNA (sgRNA).
   Clause 49B. The nucleic acid of any one of clauses 38 to 48, wherein the RNA-guided endonuclease is a Csm1 endonuclease, optionally wherein each gRNA is a single-guide RNA (sgRNA).
   Clause 50. The nucleic acid of any one of clauses 38 to 49, wherein each target sequence is located 50 to 500 base pairs (e.g., 50 to 500, 50 to 400, 50 to 300, 50 to 200, 50 to 100, 100 to 500, 100 to 400, 100 to 300, 100 to 200, 200 to 500, 200 to 400, or 200 to 300 base pairs) away from at least one other target sequence.
   Clause 51. The nucleic acid of any one of clauses 38 to 50, wherein the expression cassette contains a constitutive promoter (e.g., a CaMV 35s promoter. a maize U6 promoter, a rice U6 promoter, or a maize Ubiquitin promoter).
   Clause 52. The nucleic acid of any one of clauses 38 to 51, wherein the nucleic acid is a vector (e.g., a plasmid).
   Clause 53. The nucleic acid of any one of clauses 38 to 52, wherein the plant is a crop plant (e.g., a tomato or maize plant).
   Clause 54. The nucleic acid of any one of clauses 38 to 53, wherein the nucleic acid is contained within a cell.
   Clause 55. The nucleic acid of clause 54, wherein the cell is a plant cell (e.g., a crop plant cell), optionally wherein the cell is a non-dividing plant cell.
   Clause 56. The nucleic acid of clause 54, wherein the cell is a bacterial cell.
   Clause 57. A plant library comprising a plurality of F1 hybrid plants, each F1 hybrid plant in the plurality comprising:
(a) a gene of interest comprising a coding sequence and having a first allele that is a hypomorphic allele or a null allele and a second allele that is different from the first allele, and
(b) a CRISPR/Cas9 expression cassette that encodes a Cas9 endonuclease and at least four different guide RNAs (gRNAs), each gRNA containing a sequence that is complementary to a target sequence within a target region in the second allele of the gene of interest,
wherein the target region is 0 to 5000 base pairs upstream of the 5′ end of the coding sequence of the gene of interest or wherein the target region is 0 to 2000 base pairs downstream of the 3′ end of the coding sequence of the gene of interest.
   Clause 58. A seed library comprising a plurality of F1 hybrid seeds, each F1 hybrid seed in the plurality comprising:
(a) a gene of interest comprising a coding sequence and having a first allele that is a hypomorphic allele or a null allele and a second allele that is different from the first allele, and
(b) a CRISPR/Cas9 expression cassette that encodes a Cas9 endonuclease and at least four different guide RNAs (gRNAs), each gRNA containing a sequence that is complementary to a target sequence within a target region in the second allele of the gene of interest,
wherein the target region is 0 to 5000 base pairs upstream of the 5′ end of the coding sequence of the gene of interest or wherein the target region 0 to 2000 base pairs downstream of the 3′ end of the coding sequence of the gene of interest.
   Clause 59. The library of clause 57 or 582, wherein the target region comprises a regulatory region of the gene of interest.
   Clause 60. The library of clause 59, wherein the regulatory region comprises a transcription factor binding site, an RNA polymerase binding site, a TATA box, or a combination thereof.
   Clause 61. The library of clause 59 or 60, wherein the regulatory region is a promoter.
   Clause 62. The library of any one of clauses 57 to 61, wherein the CRISPR/Cas9 expression cassette encodes at least five different gRNAs.
   Clause 63. The library of clause 62, wherein the CRISPR/Cas9 expression cassette encodes at least six different gRNAs.
   Clause 64. The library of clause 62, wherein the CRISPR/Cas9 expression cassette encodes at least seven different gRNAs.
   Clause 65. The library of clause 62, wherein the CRISPR/Cas9 expression cassette encodes at least eight different gRNAs.
   Clause 66. The library of any one of clauses 57 to 61, wherein the CRISPR/Cas9 expression cassette encodes four to nine different gRNAs.
   Clause 67. The library of clause 66, wherein the CRISPR/Cas9 expression cassette encodes five to eight different gRNAs.
   Clause 68. The library of clause 67, wherein the CRISPR/Cas9 expression cassette encodes six to eight different gRNAs.
   Clause 69. The library of any one of clauses 57 to 68, wherein the second allele is a naturally-occurring allele.
   Clause 70. The library of any one of clauses 57 to 69, wherein the second allele is not a hypomorphic allele.
   Clause 71. The library of any one of clauses 57 to 69, wherein the second allele is not a null allele.
   Clause 72. The library of any one of clauses 57 to 71, wherein the first allele contains a mutation in a regulatory region of the gene of interest.
   Clause 73. The library of any one of clauses 57 to 71, wherein the first allele contains a mutation in a coding sequence of the gene of interest.
   Clause 74. The library of clause 72 or 73, wherein the first allele is a hypomorphic allele that results in an mRNA expression level of the gene of interest that is at least 70% lower than an allele of the gene of interest that does not contain the mutation.
   Clause 75. The library of any one of clauses 57 to 74, wherein each gRNA is a single-guide RNA (sgRNA).

Clause 76. The library of any one of clauses 57 to 75, wherein each target sequence is located 200 to 500 base pairs away from at least one other target sequence.

Clause 77. The library of any one of clauses 57 to 76, wherein the library contains at least 50 members.
Clause 78. The library of any one of clauses 57 to 77, wherein the plant or seed is a crop plant or crop seed.
Clause 79. The library of any one of clauses 57 to 78, wherein the library is a seed or plant library and at least one member of the library contains a gRNA/Cas9-induced mutation in the second allele.
Clause 80. The library of clause 79, wherein the gRNA/Cas9-induced mutation is a deletion, inversion, translocation or insertion, or a combination of structural variations thereof.
Clause 81. A method of generating a plant library comprising a plurality of F1 hybrid plants, the method comprising:
(a) providing a first plant comprising
   (i) a gene of interest comprising a coding sequence and having a first allele that is a hypomorphic allele or a null allele, and
   (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the CRISPR/Cas9 expression cassette.
   Clause 82. A method of generating a seed library comprising a plurality of F1 hybrid seeds, the method comprising:
(a) providing a first plant comprising
   (i) a gene of interest comprising a coding sequence and having a first allele that is a hypomorphic allele or a null allele, and
   (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the CRISPR/Cas9 expression cassette.
   Clause 83. The method of clauses 81 or 82, wherein the first plant is hemizygous for the CRISPR/Cas9 expression cassette.
   Clause 84. The method of any one of clauses 81 to 83, wherein the first plant is homozygous for the first allele and the second plant is homozygous for the second allele.
   Clause 85. The method of any one of clauses 81 to 84, wherein the method further comprises maintaining the plurality of F1 hybrid plants or F1 hybrid seeds under conditions that permit the gRNA/Cas9 to induce mutations within the target region of the second allele.
   Clause 86. The method of any one of clauses 81 to 85, wherein each gRNA is a single-guide RNA (sgRNA).
   Clause 87. A method of selecting members of a library having a phenotype of interest, the method comprising:
(a) providing a plant or seed library of any one of clauses 57 to 80,
(b) selecting at least one member of the library that exhibits a phenotype of interest, and
(c) crossing the at least one member to at least one plant that does not contain the CRISPR/Cas9 expression cassette.
   Clause 88. A plant or seed obtainable, or obtained by, the method of clause 87.
   Clause 89. A plant library comprising a plurality of F1 hybrid plants obtainable, or obtained by, a process comprising:
(a) providing a first plant comprising
   (i) a gene of interest comprising a coding sequence and having a first allele that is a hypomorphic allele or a null allele, and
   (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the CRISPR/Cas9 expression cassette.
   Clause 90. A seed library comprising a plurality of F1 hybrid seeds obtainable, or obtained by, a process comprising:
(a) providing a first plant comprising
   (i) a gene of interest comprising a coding sequence and having a first allele that is a hypomorphic allele or a null allele, and
   (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the CRISPR/Cas9 expression cassette.
   Clause 91. The plant or seed library of clauses 89 or 90, wherein the first plant is hemizygous for the CRISPR/Cas9 expression cassette.
   Clause 92. The plant or seed library of any one of clauses 89 to 91, wherein the first plant is homozygous for the first allele and the second plant is homozygous for the second allele.
   Clause 93. The plant or seed library of any one of clauses 89 to 92, wherein the method further comprises maintaining the plurality of F1 hybrid plants or F1 hybrid seeds under conditions that permit the gRNA/Cas9 to induce mutations within the target region of the second allele.
   Clause 94. The plant or seed library of any one of clauses 89 to 93, wherein each gRNA is a single-guide RNA (sgRNA).
   Clause 95. A plant or seed that is homozygous for a second allele of a gene of interest containing at least one gRNA/Cas9-induced mutation obtainable, or obtained by, a process comprising:
(a) providing a first plant comprising
   (i) a gene of interest comprising a coding sequence and having a first allele that is a hypomorphic allele or a null allele, and
   (ii) a CRISPR/Cas9 expression cassette that encodes a Cas9 endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest,
(c) crossing the first plant to the second plant to produce a plurality of F1 hybrid plants, each F1 hybrid plant in the plurality comprising the first allele, the second allele and the CRISPR/Cas9 expression cassette,
(d) maintaining the plurality of F1 hybrid plants under conditions that permit the gRNA/Cas9 to induce mutations within the target region of the second allele,
(e) selecting an F1 hybrid plant of step (d) having a phenotype of interest, and
(f) performing a cross with the selected F1 hybrid plant to produce a progeny plant or seed that is homozygous for the second allele containing at least one gRNA/Cas9-induced mutation.
   Clause 96. The plant or seed of clause 95, wherein the mutation is a deletion, inversion, translocation or insertion, or a combination of structural variations thereof.
   Clause 97. A plant cell or seed cell obtainable, or obtained by, a process comprising isolating a cell from the plant or seed of clause 94 or 95.
   Clause 98. An isolated DNA molecule comprising a second allele of a gene of interest containing at least one gRNA/Cas9-induced mutation or a fragment of the second allele containing the target region containing the at least one gRNA/Cas9-induced mutation, the DNA molecule obtainable, or obtained by, a process comprising isolating a DNA molecule comprising the second allele, or the fragment thereof, from the plant or seed of clause 95 or 96 or from the plant cell or seed cell of clause 97.

### EXAMPLES

### Example 1: Mutagenesis Strategy for Creating Weak Alleles

Changes to gene regulation have been major drivers in crop domestication, and in evolution more broadly (King et al 1975 and Olsen et al 2013). QTL and GWAS studies on crop plants over the last two decades have revealed that nearly half of all changes identified as important in domestication genes are cis-regulatory. Many of these likely alter expression, and have weak but beneficial phenotypic effects, as opposed to the often deleterious effects of null alleles (Doebley 2006 and Meyer et al 2013). An example of a cis-regulatory mutant is tomato fas, which displays reduced SlCLV3 expression (Xu et al 2015). It is thought that changes to cis-regulatory elements have less potential for negative pleiotropy than changes to protein structure (Carroll et al 2000, Carroll et al 2008 and Stern 2000). A major reason for this is the modular organization and inherent redundancy that exists in promoters. Evidence from animals suggests that such redundancy provides robustness for gene expression, particularly under perturbation (Frankel et al 2010).

cis-regulatory elements in gene promoters present an exciting target for creating new, weak alleles, with the ultimate goal of modulating crop yield traits. As described in Example 2 below, a construct containing Cas9 and a series of guide RNAs that target regulatory regions can be used to induce CRISPR/Cas9-mediated mutations in regulatory regions that create collections of novel expression alleles and networks directly linked to crop productivity that can provide a powerful new source of genetic diversity for breeding. The CLV signaling network (Bommert et al and Xu et al 2015) was used to test this hypothesis in tomatoes as described in Example 2 below. Similar tests are performed in maize. Arabidopsis has the ability to quickly generate T1 transgenic lines at little cost and the power to rapidly establish T2 populations and screen thousands of plants in minimal space. Thus, Arabidopsis provides a fast, in-depth path to optimize identification and characterization of CRISPR/Cas9-generated promoter alleles, which, in some embodiments, can be used to further guide experiments in maize and tomato.

Promoter Analysis.

In some embodiments, it may be useful to predict which sequence changes, outside of protein coding space, might yield phenotypes. Three markers that might signify a useful promoter region are: (1) transcription factor binding sites, (2) conserved non-coding sequences (CNSs), and (3) reduced SNP density. These markers are not mutually exclusive, for example, a CNS, and/or reduced SNP density may signify an as-yet uncategorized transcription factor binding site. In other embodiments, a defined region upstream of the transcription start site of a coding sequence (e.g., within 5 kb), which is likely to contain such regulatory sequences, can be targeted without first assessing those regions for the presence of transcription factor binding sites, CNSs, or reduced SNP density.

First, promoter regions of CLV network genes, including WUS homologs, are analyzed from Arabidopsis, maize, and tomato. The promoter sequences 3-4 kb upstream of transcription start sites are analyzed using existing databases of transcription factor binding sites and plant CNSs (see, e.g., Sandelin et al 2004, Turco et al 2013, O'Connor et al 2005, Baxter et al 2012, Haudry et al 2013 and Matys et al 2003). Novel CNSs may also be identified in available Solanaceae genomes (S. lycopersicum, S. pimpinellifolium, S. pennellii, S. tuberosum, C. annuum, N. benthamiana) using a CNS discovery pipeline for recently diverged genomes (Turco et al 2013). For wider searches between families, the DREME discriminatory motif search tool (Bailey 2011) may be used to identify motifs present in one orthology group, but not another, and to find motifs present in promoter regions, but not in distal, unrelated DNA sequence.

SNP datasets from all three species are used to identify regions of reduced SNP density in promoters, using established methods (Korkuc et al 2014, Chia et al 2012, and Sim et al 2012). Novel motifs identified from the above-described strategies are searched for in all promoters of interest. It is expected that gene copies involved in responsive backup circuits will share some, but not all, motifs and TF binding sites (Kafri et al 2005). Evidence of CNSs or TF binding regions shared between gene clades and/or species may become high-priority regions to inform promoter-targeting experiments.

Cas9 Targeting of Promoters.

As demonstrated with feat and fea3 in maize (Bommert et al 2013 and Je at al 2016), TILLING for coding region mutations can provide beneficial weak alleles for breeding; however, this approach is time-consuming and inefficient (Till et al 2004). CRISPR/Cas9 opens up the opportunity to design a novel approach to specifically target promoter regions. The promoters of CLV network genes are targeted, such as described in Example 2 below. The promoters for CLV1, 2 and 3, and potential homologs with redundant functions are targeted. WUS regulatory elements are also targeted in all three species, but focused on the 3′ region, where there is evidence from tomato that the lc mutation is caused by CNS polymorphisms 1.9 kb downstream of S/WUS (Munos et al 2011 and van der Knaap et al 2014).

In order to target these regions, two CRISPR/Cas9 constructs are generated, each containing 8 sgRNAs that target proximal and distal promoter regions of each gene in arabidopsis, maize, and tomato. The target site selection may be guided by promoter analyses as described above. This will reveal motifs with potential cis-regulatory function, but may also or alternatively include even spacing to cover the entire region. Selected target sites are cross-referenced with the CRISPR-P web portal to select sgRNAs that have few or no matches elsewhere (Lei et al 2014). Importantly, the high frequency of PAM sites (NGG) genome-wide will provide for multiple targets within each promoter.

As described herein, such as in Example 2 below, use of two sgRNAs in a single Cas9 construct can result in a range of mutation events. For example, in one set of forty-five T2 Arabidopsis plants containing Cas9 and dual gRNA target sites spaced 30 bp apart, fourteen different allele types were found, ranging from single nucleotide indel events, to complete deletions, to hybrid indel events, and even inversions between two gRNA target sites that leave the flanking genomic DNA intact. As such, it is expected that using Cas9 with two or more gRNA will generate alleles with large portions of promoters deleted, as well as alleles that are peppered with multiple small and/or large indels throughout the target region. The power of this approach therefore lies in the wide range of alleles that should be generated by targeting promoter regulatory element redundancy (Wray et al 2003, Rombauts et al 2003, and Paixao et al 2010). It is anticipated that the collection of alleles produced using this approach will result in a quantitative range of modifications on meristem homeostasis and yield traits, akin to QTL such as lc and fas in tomato (Xu et al 2015), without the offsets associated with strong null alleles (Bommert et al 2013).

Even more, to augment the collection of alleles, T1 transgenics targeting proximal and distal promoter regions for each gene are crossed together, to bring together transgenes to express 16 sgRNAs simultaneously. The resulting mutational promiscuity and diversity is expected to generate an allelic series that can provide weak, moderate, and strong phenotypic effects. Such diversity is shown, for example, in Example 2 below.

Phenotyping and Molecular Analysis.

Using the near-random nature of CRISPR/Cas9 mutagenesis as an advantage, an unbiased approach is used to identify plants carrying desirable promoter mutations. Specifically, multiple independent T1 plants are generated for each species and T2 progeny are screened for individuals with enhanced meristem size, as determined by changes in phenotype resembling weak fasciation. Because most T1 plants will be chimeric, it is anticipated that a large array of allelic forms will be transmitted. Therefore, at least 200 T2 progeny are screened each from a minimum of ten T1 plants.

In some embodiments, weak effects on phenotype are desired; however, all levels of phenotype are assessed, including strong fasciation, in order to characterize functional cis-regulatory elements that can be validated through molecular analyses of promoter alleles. In Arabidopsis, this involves identifying plants showing typical clv mutant fasciation, which includes thickened and fused stems and more flowers with extra organs. To isolate weak alleles, plants are identified that produce shorter siliques with additional carpels, but are otherwise normal. Likewise, in tomato, it may be desirable to screen for increased inflorescence branching and fasciated flowers, but the focus may be on identifying milder individuals with extra floral organs and larger fruits. In maize, it may be desirable to screen for ear and/or tassel fasciation, and plants showing subtle increases in kernel row number or spikelet density. Large populations of T2 progeny are screened in growth chambers and greenhouses for Arabidopsis, and in fields for maize and tomato. Plants displaying fasciation or enhanced yield traits are grouped according to phenotypic strength, and the promoters from each individual are sequenced. Leaves from different regions of the plants are pooled, allowing for identification of homozygous stable promoter mutants. Select individuals are outcrossed to non-transgenic plants to segregate away the transgene and recover stable promoter variants.

One potential complication of this approach is that fasciated T2 progeny could be biallelic, for example carrying one weak and one strong allele, or even chimeric, if Cas9 is maintained. Thus, the phenotypic effect from a homozygous allele should be evaluated in T3 plants. If simply selfed, ¾ of the T2 plants will retain the Cas9 transgene, potentially leading to new mutation events that could further disrupt putative weak alleles, converting them into strong alleles. These potential issues are less of a concern for Arabidopsis, where size and generation time allow large-scale screening in T3 and later generations. To address these potential issues in tomato and maize, a parallel screen is performed in which T1 transgenics are outcrossed to corresponding null mutant tester lines. For example, T1 plants targeting the tomato SlCLV3 promoter are outcrossed to stable homozygous null CR-Slclv3 mutants, which are recessive. The sensitized background allows for rapid selection of mutated promoter alleles that cause a change in expression, and should facilitate identification of the most desirable weak alleles, since a weak allele in the presence of a null allele may provide a more obvious phenotype. The SlCLV3 promoter from selected F1 plants is then sequenced as above to determine allele type, and F2 progeny from these same plants are screened to isolate lines that are homozygous for weak alleles. An added benefit of this approach is that half of the outcrossed F1 progeny will no longer carry Cas9, assuming a single insertion event. Advantageously, the above approach requires little effort in order to obtain sufficient F1 seed for tomato and maize, and at least 200 F1 seed are generated from each of five T1 plants that are also self pollinated for screening as outlined above. Null alleles of CLV1, 2, and 3 are already available for tomato as well as for maize td1 (clv1) and feat (c1v2). A null allele of maize CLV3 is produced using Cas9-targeting of the coding sequence.

Once stable promoter mutants are obtained, vegetative and inflorescence meristem size alterations are precisely quantified (e.g. by SEM (Taguchi-Shiobara et al 2001, Bommert et al 2013, Xu et al 2015, Nimchuk et al 2015 and Park et al 2012)) for each promoter variant in each species to create a comparative dataset of the different promoter requirements. This promoter analysis is mapped onto regulatory motif predictions and functional cis-regulatory elements are identified that are conserved or species-specific. The expression changes in the gene controlled by the promoter are then analyzed in selected lines by qRT-PCR or in situ hybridization. Functional elements may provide a dataset that may inform future studies aimed at identifying trans-acting factors. In this respect, Arabidopsis is useful, as it allows for rapid confirmation of the function of predicted cis-regulatory elements. It is also anticipated that weak promoter alleles will create sensitized backgrounds for genetic analysis of plant development in all three systems. As such, this study may provide a large-scale functional test of identified CNS elements in plant genomes, generate datasets and resources for functional analyses, and create valuable novel crop plant alleles that affect meristem homeostasis to improve agronomic traits for breeding.

### Example 2: Generation of Quantitative Trait Variation for Crop Improvement Using CRISPR/Cas9 Gene-Editing

### Abstract

Crop improvement refers to the systematic process of selection for desirable traits, both qualitative and quantitative, relying on rather limited sources of naturally occurring genetic variation affecting both coding sequence and regulatory regions. As described herein, the power of gene editing via CRISPR/Cas9 technology was harnessed, through the implementation of a reverse/forward genetic screen, to generate new sources of quantitative phenotypic variation for fruit size and shoot architecture in tomato, by engineering transcriptional alleles carrying induced mutations in regulatory regions. This approach reveals the power of gene editing to create new sources of genetic variation in a controlled and directed manner, providing a useful and potentially revolutionary tool for boosting crop improvement.

### Methods

### Generation of the CRISPR/Cas9 Expression Cassette for SlCLV3 Promoter Targeting

A binary vector containing a CRISPR cassette with a functional Cas9 under a constitutive promoter and eight single-guide RNAs (sgRNAs) was made using a standard protocol of Golden Gate assembly (Werner et al., 2012; Brooks et al., 2014). First, eight potential 20 base pair (bp) sites were selected for sgRNA design within a region of 2000 bp upstream of the transcriptional start site (TSS) of SlCLV3 (Solyc11g071380) using the CRISPR-P tool (Lei et al., 2014). These sgRNA sequences were cloned downstream of the Arabidopsis thaliana U6 promoter to produce individual sgRNA expression cassettes. Each sgRNA was cloned individually into the level 1 vectors pICH47732 (sgRNA1 or sgRNA8), pICH47742 (sgRNA2), pICH47751 (sgRNA3), pICH47761 (sgRNA4), pICH47772 (sgRNA5), pICH47781 (sgRNA6), pICH47791 (sgRNA7). Subsequently, sgRNAs were assembled into two groups in an intermediate cloning step, using level M vectors pAGM8055 and pAGM8093. Level 1 constructs pICH47732-NOSpro::NPTII (selection maker), pICH47742-35S:Cas9 constructs and level M vectors containing the cloned sgRNAs were then assembled in the binary Level 2 vector pAGM4723. All restriction-ligation Golden Gate reactions were carried out in a volume of 15 μL in a thermal cycler (3 min at 37° C. and 4 min at 16° for 20 cycles; 5 min at 50° C., 5 min at 80° C., and final storage at 4° C.). The same methodology described above was used to generate a CRISPR/Cas9 expression cassette for targeting a region upstream of the transcriptional start site (TSS) of the SELF PRUNING (SP) gene.

### Annotated PAGM4723 Sequence:

CaMV 2x35s promoter: 1904-2656 bp
Cas9: 2743-6883 bp
sgRNA1 guide sequence: 7250-7269 bp
sgRNA1 scaffold sequence: 7270-7345 bp
sgRNA2 guide sequence: 7486-7505 bp
sgRNA2 scaffold sequence: 7506-7581 bp
sgRNA3 guide sequence: 7722-7741 bp
sgRNA3 scaffold sequence: 7742-7817 bp
sgRNA4 guide sequence: 7958-7977 bp
sgRNA4 scaffold sequence: 7978-8053 bp
sgRNA5 guide sequence: 8194-8213 bp
sgRNA5 scaffold sequence: 8214-8289 bp
sgRNA6 guide sequence: 8431-8450 bp
sgRNA6 scaffold sequence: 8451-8526 bp
sgRNA7 guide sequence: 8667-8686 bp
sgRNA7 scaffold sequence: 8687-8762 bp
sgRNA8 guide sequence: 8903-8922 bp
sgRNA8 scaffold sequence: 8923-8998 bp
(SEQ ID NO: 2)
GTGCCGAATTCGGATCCGGAGCGGAGAATTAAGGGAGTCACGTTATGACCCCCG
CCGATGACGCGGGACAAGCCGTTTTACGTTTGGAACTGACAGAACCGCAACGAT
TGAAGGAGCCACTCAGCCGCGGGTTTCTGGAGTTTAATGAGCTAAGCACATACGT
CAGAAACCATTATTGCGCGTTCAAAAGTCGCCTAAGGTCACTATCAGCTAGCAAA
TATTTCTTGTCAAAAATGCTCCACTGACGTTCCATAAATTCCCCTCGGTATCCAAT
TAGAGTCTCATATTCACTCGACTTTTACAACAATTACCAACAACAACAAACAACA
AACAACATTACAATTACTATTTACAATTATCCATGGTTGAACAAGATGGATTGCA
CGCAGGTTCTCCGGCCGCTTGGGTGGAGAGGCTATTCGGCTATGACTGGGCACAA
CAGACAATCGGCTGCTCTGATGCCGCCGTGTTCCGGCTGTCAGCGCAGGGGCGCC
CGGTTCTTTTTGTCAAGACCGACCTGTCCGGTGCCCTGAATGAACTGCAGGACGA
GGCAGCGCGGCTATCGTGGCTGGCCACGACGGGCGTTCCTTGCGCAGCTGTGCTC
GACGTTGTCACTGAAGCGGGAAGGGACTGGCTGCTATTGGGCGAAGTGCCGGGG
CAGGATCTCCTGTCATCTCACCTTGCTCCTGCCGAGAAAGTATCCATCATGGCTG
ATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGC
CAGGCTCAAGGCGCGCATGCCCGACGGCGAGGATCTCGTCGTGACTCATGGCGA
TGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTG
ATATTGCTGAAGAGCTTGGCGGCGAATGGGCTGACCGCTTCCTCGTGCTTTACGG
TATCGCCGCTCCCGATTCGCAGCGCATCGCCTTCTATCGCCTTCTTGACGAGTTCT
TCTGAGCGGGACTCTGGGGTTCGCTGCTTTAATGAGATATGCGAGACGCCTATGA
TCGCATGATATTTGCTTTCAATTCTGTTGTGCACGTTGTAAAAAACCTGAGCATGT
GTAGCTCAGATCCTTACCGCCGGTTTCGGTTCATTCTAATGAATATATCACCCGTT
ACTATCGTATTTTTATGAATAATATTCTCCGTTCAATTTACTGATTGTACCCTACT
ACTTATATGTACAATATTAAAATGAAAACAATATATTGTGCTGAATAGGTTTATA
GCGACATCTATGATAGAGCGCCACAATAACAAACAATTGCGTTTTATTATTACAA
ATCCAATTTTAAAAAAAGCGGCAGAACCGGTCAAACCTAAAAGACTGATTACAT
AAATCTTATTCAAATTTCAAAAGGCCCCAGGGGCTAGTATCTACGACACACCGAG
CGGCGAACTAATAACGTTCACTGAAGGGAACTCCGGTTCCCCGCCGGCGCGCAT
GGGTGAGATTCCTTGAAGTTGAGTATTGGCCGTCCGCTCTACCGAAAGTTACGGG
CACCATTCAACCCGGTCCAGCACGGCGGCCGGGTAACCGACTTGCTGCCCCGAG
AATTATGCAGCATTTTTTTGGTGTATGTGGGCCCCAAATGAAGTGCAGGTCAAAC
CTTGACAGTGACGACAAATCGTTGGGCGGGTCCAGGGCGAATTTTGCGACAACA
TGTCGAGGCTCAGCCGCTGCAAGAATTCAAGCTTGGAGGTCAACATGGTGGAGC
ACGACACTCTGGTCTACTCCAAAAATGTCAAAGATACAGTCTCAGAAGATCAAA
GGGCTATTGAGACTTTTCAACAAAGGATAATTTCGGGAAACCTCCTCGGATTCCA
TTGCCCAGCTATCTGTCACTTCATCGAAAGGACAGTAGAAAAGGAAGGTGGCTC
CTACAAATGCCATCATTGCGATAAAGGAAAGGCTATCATTCAAGATCTCTCTGCC
GACAGTGGTCCCAAAGATGGACCCCCACCCACGAGGAGCATCGTGGAAAAAGAA
GAGGTTCCAACCACGTCTACAAAGCAAGTGGATTGATGTGATAACATGGTGGAG
CACGACACTCTGGTCTACTCCAAAAATGTCAAAGATACAGTCTCAGAAGATCAA
AGGGCTATTGAGACTTTTCAACAAAGGATAATTTCGGGAAACCTCCTCGGATTCC
ATTGCCCAGCTATCTGTCACTTCATCGAAAGGACAGTAGAAAAGGAAGGTGGCT
CCTACAAATGCCATCATTGCGATAAAGGAAAGGCTATCATTCAAGATCTCTCTGC
CGACAGTGGTCCCAAAGATGGACCCCCACCCACGAGGAGCATCGTGGAAAAAGA
AGAGGTTCCAACCACGTCTACAAAGCAAGTGGATTGATGTGACATCTCCACTGAC
GTAAGGGATGACGCACAATCCCACTATCCTTCGCAAGACCCTTCCTCTATATAAG
GAAGTTCATTTCATTTGGAGAGGACACGCTCGAGTATAAGAGCTCATTTTTACAA
CAATTACCAACAACAACAAACAACAAACAACATTACAATTACATTTACAATTATC
GATACAATGGACAAGAAGTACTCCATTGGGCTCGATATCGGCACAAACAGCGTC
GGCTGGGCCGTCATTACGGACGAGTACAAGGTGCCGAGCAAAAAATTCAAAGTT
CTGGGCAATACCGATCGCCACAGCATAAAGAAGAACCTCATTGGCGCCCTCCTGT
TCGACTCCGGGGAGACGGCCGAAGCCACGCGGCTCAAAAGAACAGCACGGCGC
AGATATACCCGCAGAAAGAATCGGATCTGCTACCTGCAGGAGATCTTTAGTAAT
GAGATGGCTAAGGTGGATGACTCTTTCTTCCATAGGCTGGAGGAGTCCTTTTTGG
TGGAGGAGGATAAAAAGCACGAGCGCCACCCAATCTTTGGCAATATCGTGGACG
AGGTGGCGTACCATGAAAAGTACCCAACCATATATCATCTGAGGAAGAAGCTTG
TAGACAGTACTGATAAGGCTGACTTGCGGTTGATCTATCTCGCGCTGGCGCATAT
GATCAAATTTCGGGGACACTTCCTCATCGAGGGGGACCTGAACCCAGACAACAG
CGATGTCGACAAACTCTTTATCCAACTGGTTCAGACTTACAATCAGCTTTTCGAA
GAGAACCCGATCAACGCATCCGGAGTTGACGCCAAAGCAATCCTGAGCGCTAGG
CTGTCCAAATCCCGGCGGCTCGAAAACCTCATCGCACAGCTCCCTGGGGAGAAG
AAGAACGGCCTGTTTGGTAATCTTATCGCCCTGTCACTCGGGCTGACCCCCAACT
TTAAATCTAACTTCGACCTGGCCGAAGATGCCAAGCTTCAACTGAGCAAAGACA
CCTACGATGATGATCTCGACAATCTGCTGGCCCAGATCGGCGACCAGTACGCAG
ACCTTTTTTTGGCGGCAAAGAACCTGTCAGACGCCATTCTGCTGAGTGATATTCT
GCGAGTGAACACGGAGATCACCAAAGCTCCGCTGAGCGCTAGTATGATCAAGCG
CTATGATGAGCACCACCAAGACTTGACTTTGCTGAAGGCCCTTGTCAGACAGCAA
CTGCCTGAGAAGTACAAGGAAATTTTCTTCGATCAGTCTAAAAATGGCTACGCCG
GATACATTGACGGCGGAGCAAGCCAGGAGGAATTTTACAAATTTATTAAGCCCA
TCTTGGAAAAAATGGACGGCACCGAGGAGCTGCTGGTAAAGCTTAACAGAGAAG
ATCTGTTGCGCAAACAGCGCACTTTCGACAATGGAAGCATCCCCCACCAGATTCA
CCTGGGCGAACTGCACGCTATCCTCAGGCGGCAAGAGGATTTCTACCCCTTTTTG
AAAGATAACAGGGAAAAGATTGAGAAAATCCTCACATTTCGGATACCCTACTAT
GTAGGCCCCCTCGCCCGGGGAAATTCCAGATTCGCGTGGATGACTCGCAAATCA
GAAGAGACTATCACTCCCTGGAACTTCGAGGAAGTCGTGGATAAGGGGGCCTCT
GCCCAGTCCTTCATCGAAAGGATGACTAACTTTGATAAAAATCTGCCTAACGAAA
AGGTGCTTCCTAAACACTCTCTGCTGTACGAGTACTTCACAGTTTATAACGAGCT
CACCAAGGTCAAATACGTCACAGAAGGGATGAGAAAGCCAGCATTCCTGTCTGG
AGAGCAGAAGAAAGCTATCGTGGACCTCCTCTTCAAGACGAACCGGAAAGTTAC
CGTGAAACAGCTCAAAGAAGATTATTTCAAAAAGATTGAATGTTTCGACTCTGTT
GAAATCAGCGGAGTGGAGGATCGCTTCAACGCATCCCTGGGAACGTATCACGAT
CTCCTGAAAATCATTAAAGACAAGGACTTCCTGGACAATGAGGAGAACGAGGAC
ATTCTTGAGGACATTGTCCTCACCCTTACGTTGTTTGAAGATAGGGAGATGATTG
AAGAACGCTTGAAAACTTACGCTCATCTCTTCGACGACAAAGTCATGAAACAGCT
CAAGAGGCGCCGATATACAGGATGGGGGCGGCTGTCAAGAAAACTGATCAATGG
GATCCGAGACAAGCAGAGTGGAAAGACAATCCTGGATTTTCTTAAGTCCGATGG
ATTTGCCAACCGGAACTTCATGCAGTTGATCCATGATGACTCTCTCACCTTTAAG
GAGGACATCCAGAAAGCACAAGTTTCTGGCCAGGGGGACAGTCTCCACGAGCAC
ATCGCTAATCTTGCAGGTAGCCCAGCTATCAAAAAGGGAATACTGCAGACCGTT
AAGGTCGTGGATGAACTCGTCAAAGTAATGGGAAGGCATAAGCCCGAGAATATC
GTTATCGAGATGGCCCGAGAGAACCAAACTACCCAGAAGGGACAGAAGAACAG
TAGGGAAAGGATGAAGAGGATTGAAGAGGGTATAAAAGAACTGGGGTCCCAAA
TCCTTAAGGAACACCCAGTTGAAAACACCCAGCTTCAGAATGAGAAGCTCTACCT
GTACTACCTGCAGAACGGCAGGGACATGTACGTGGATCAGGAACTGGACATCAA
TCGGCTCTCCGACTACGACGTGGATCATATCGTGCCCCAGTCTTTTCTCAAAGAT
GATTCTATTGATAATAAAGTGTTGACAAGATCCGATAAAAATAGAGGGAAGAGT
GATAACGTCCCCTCAGAAGAAGTTGTCAAGAAAATGAAAAATTATTGGCGGCAG
CTGCTGAACGCCAAACTGATCACACAACGGAAGTTCGATAATCTGACTAAGGCT
GAACGAGGTGGCCTGTCTGAGTTGGATAAAGCCGGCTTCATCAAAAGGCAGCTT
GTTGAGACACGCCAGATCACCAAGCACGTGGCCCAAATTCTCGATTCACGCATG
AACACCAAGTACGATGAAAATGACAAACTGATTCGAGAGGTGAAAGTTATTACT
CTGAAGTCTAAGCTGGTTTCAGATTTCAGAAAGGACTTTCAGTTTTATAAGGTGA
GAGAGATCAACAATTACCACCATGCGCATGATGCCTACCTGAATGCAGTGGTAG
GCACTGCACTTATCAAAAAATATCCCAAGCTTGAATCTGAATTTGTTTACGGAGA
CTATAAAGTGTACGATGTTAGGAAAATGATCGCAAAGTCTGAGCAGGAAATAGG
CAAGGCCACCGCTAAGTACTTCTTTTACAGCAATATTATGAATTTTTTCAAGACC
GAGATTACACTGGCCAATGGAGAGATTCGGAAGCGACCACTTATCGAAACAAAC
GGAGAAACAGGAGAAATCGTGTGGGACAAGGGTAGGGATTTCGCGACAGTCCG
GAAGGTCCTGTCCATGCCGCAGGTGAACATCGTTAAAAAGACCGAAGTACAGAC
CGGAGGCTTCTCCAAGGAAAGTATCCTCCCGAAAAGGAACAGCGACAAGCTGAT
CGCACGCAAAAAAGATTGGGACCCCAAGAAATACGGCGGATTCGATTCTCCTAC
AGTCGCTTACAGTGTACTGGTTGTGGCCAAAGTGGAGAAAGGGAAGTCTAAAAA
ACTCAAAAGCGTCAAGGAACTGCTGGGCATCACAATCATGGAGCGATCAAGCTT
CGAAAAAAACCCCATCGACTTTCTCGAGGCGAAAGGATATAAAGAGGTCAAAAA
AGACCTCATCATTAAGCTTCCCAAGTACTCTCTCTTTGAGCTTGAAAACGGCCGG
AAACGAATGCTCGCTAGTGCGGGCGAGCTGCAGAAAGGTAACGAGCTGGCACTG
CCCTCTAAATACGTTAATTTCTTGTATCTGGCCAGCCACTATGAAAAGCTCAAAG
GATCTCCCGAAGATAATGAGCAGAAGCAGCTGTTCGTGGAACAACACAAACACT
ACCTTGATGAGATCATCGAGCAAATAAGCGAATTCTCCAAAAGAGTGATCCTCG
CCGACGCTAACCTCGATAAGGTGCTTTCTGCTTACAATAAGCACAGGGATAAGCC
CATCAGGGAGCAGGCAGAAAACATTATCCACTTGTTTACTCTGACCAACTTGGGC
GCGCCTGCAGCCTTCAAGTACTTCGACACCACCATAGACAGAAAGCGGTACACC
TCTACAAAGGAGGTCCTGGACGCCACACTGATTCATCAGTCAATTACGGGGCTCT
ATGAAACAAGAATCGACCTCTCTCAGCTCGGTGGAGACAGCAGGGCTGACCCCA
AGAAGAAGAGGAAGGTGTGAGCTTGTCAAGCAGATCGTTCAAACATTTGGCAAT
AAAGTTTCTTAAGATTGAATCCTGTTGCCGGTCTTGCGATGATTATCATATAATTT
CTGTTGAATTACGTTAAGCATGTAATAATTAACATGTAATGCATGACGTTATTTA
TGAGATGGGTTTTTATGATTAGAGTCCCGCAATTATACATTTAATACGCGATAGA
AAACAAAATATAGCGCGCAAACTAGGATAAATTATCGCGCGCGGTGTCATCTAT
GTTACTAGATCGACGCTACTAGAATTCGAGCTCGGAGTGATCAAAAGTCCCACAT
CGATCAGGTGATATATAGCAGCTTAGTTTATATAATGATAGAGTCGACATAGCGA
TTGATATACAACAATGGCTGCAGTTTTAGAGCTAGAAATAGCAAGTTAAAATAA
GGCTAGTCCGTTATCAACTTGAAAAAGTGGCACCGAGTCGGTGCTTTTTTTCTAG
ACCCAGCTTTCTTGTACAAAGTTGGCATTACGCTTTACGAATTCCCATGGGGAGT
GATCAAAAGTCCCACATCGATCAGGTGATATATAGCAGCTTAGTTTATATAATGA
TAGAGTCGACATAGCGATTGACCTTATCCCCTGCCTTTAGTTTTAGAGCTAGAAA
TAGCAAGTTAAAATAAGGCTAGTCCGTTATCAACTTGAAAAAGTGGCACCGAGT
CGGTGCTTTTTTTCTAGACCCAGCTTTCTTGTACAAAGTTGGCATTACGCTCAGAG
AATTCGCATGCGGAGTGATCAAAAGTCCCACATCGATCAGGTGATATATAGCAG
CTTAGTTTATATAATGATAGAGTCGACATAGCGATTGAAACACCAAATTATGTTG
TGTTTTAGAGCTAGAAATAGCAAGTTAAAATAAGGCTAGTCCGTTATCAACTTGA
AAAAGTGGCACCGAGTCGGTGCTTTTTTTCTAGACCCAGCTTTCTTGTACAAAGT
TGGCATTACGCTTGTGGAATTCCTCGAGGGAGTGATCAAAAGTCCCACATCGATC
AGGTGATATATAGCAGCTTAGTTTATATAATGATAGAGTCGACATAGCGATTGAG
ATCCATAGTACAGTACTGTTTTAGAGCTAGAAATAGCAAGTTAAAATAAGGCTA
GTCCGTTATCAACTTGAAAAAGTGGCACCGAGTCGGTGCTTTTTTTCTAGACCCA
GCTTTCTTGTACAAAGTTGGCATTACGCTGAGCGAATTCCATATGGGAGTGATCA
AAAGTCCCACATCGATCAGGTGATATATAGCAGCTTAGTTTATATAATGATAGAG
TCGACATAGCGATTGCAGTAACAAGACAGAGTGAGTTTTAGAGCTAGAAATAGC
AAGTTAAAATAAGGCTAGTCCGTTATCAACTTGAAAAAGTGGCACCGAGTCGGT
GCTTTTTTTCTAGACCCAGCTTTCTTGTACAAAGTTGGCATTACGCTTGCCGAATT
CGGATCCGGAGTGATCAAAAGTCCCACATCGATCAGGTGATATATAGCAGCTTA
GTTTATATAATGATAGAGTCGACATAGCGATTGGTCCAACAATATATGTTTATGT
TTTAGAGCTAGAAATAGCAAGTTAAAATAAGGCTAGTCCGTTATCAACTTGAAA
AAGTGGCACCGAGTCGGTGCTTTTTTTCTAGACCCAGCTTTCTTGTACAAAGTTG
GCATTACGCTGCAAGAATTCAAGCTTGGAGTGATCAAAAGTCCCACATCGATCA
GGTGATATATAGCAGCTTAGTTTATATAATGATAGAGTCGACATAGCGATTGACA
CCACTCGATTTAAATTGTTTTAGAGCTAGAAATAGCAAGTTAAAATAAGGCTAGT
CCGTTATCAACTTGAAAAAGTGGCACCGAGTCGGTGCTTTTTTTCTAGACCCAGC
TTTCTTGTACAAAGTTGGCATTACGCTACTAGAATTCGAGCTCGGAGTGATCAAA
AGTCCCACATCGATCAGGTGATATATAGCAGCTTAGTTTATATAATGATAGAGTC
GACATAGCGATTGCAATGCAAGTAGCTGCAAAGTTTTAGAGCTAGAAATAGCAA
GTTAAAATAAGGCTAGTCCGTTATCAACTTGAAAAAGTGGCACCGAGTCGGTGCT
TTTTTTCTAGACCCAGCTTTCTTGTACAAAGTTGGCATTACGCTTTACGAGGATGC
ACATGTGACCGAGGGACACGAAGTGATCCGTTTAAACTATCAGTGTTTGACAGG
ATATATTGGCGGGTAAACCTAAGAGAAAAGAGCGTTTATTAGAATAATCGGATA
TTTAAAAGGGCGTGAAAAGGTTTATCCGTTCGTCCATTTGTATGTGCCAGCCGTG
CGGCTGCATGAAATCCTGGCCGGTTTGTCTGATGCCAAGCTGGCGGCCTGGCCGG
CCAGCTTGGCCGCTGAAGAAACCGAGCGCCGCCGTCTAAAAAGGTGATGTGTAT
TTGAGTAAAACAGCTTGCGTCATGCGGTCGCTGCGTATATGATGCGATGAGTAAA
TAAACAAATACGCAAGGGGAACGCATGAAGGTTATCGCTGTACTTAACCAGAAA
GGCGGGTCAGGCAAGACGACCATCGCAACCCATCTAGCCCGCGCCCTGCAACTC
GCCGGGGCCGATGTTCTGTTAGTCGATTCCGATCCCCAGGGCAGTGCCCGCGATT
GGGCGGCCGTGCGGGAAGATCAACCGCTAACCGTTGTCGGCATCGACCGCCCGA
CGATTGACCGCGACGTGAAGGCCATCGGCCGGCGCGACTTCGTAGTGATCGACG
GAGCGCCCCAGGCGGCGGACTTGGCTGTGTCCGCGATCAAGGCAGCCGACTTCG
TGCTGATTCCGGTGCAGCCAAGCCCTTACGACATATGGGCCACCGCCGACCTGGT
GGAGCTGGTTAAGCAGCGCATTGAGGTCACGGATGGAAGGCTACAAGCGGCCTT
TGTCGTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCGTGAGC
TACCCAGGCACTGCCGCCGCCGGCACAACCGTTCTTGAATCAGAACCCGAGGGC
GACGCTGCCCGCGAGGTCCAGGCGCTGGCCGCTGAAATTAAATCAAAACTCATTT
GAGTTAATGAGGTAAAGAGAAAATGAGCAAAAGCACAAACACGCTAAGTGCCG
GCCGTCCGAGCGCACGCAGCAGCAAGGCTGCAACGTTGGCCAGCCTGGCAGACA
CGCCAGCCATGAAGCGGGTCAACTTTCAGTTGCCGGCGGAGGATCACACCAAGC
TGAAGATGTACGCGGTACGCCAAGGCAAGACCATTACCGAGCTGCTATCTGAAT
ACATCGCGCAGCTACCAGAGTAAATGAGCAAATGAATAAATGAGTAGATGAATT
TTAGCGGCTAAAGGAGGCGGCATGGAAAATCAAGAACAACCAGGCACCGACGC
CGTGGAATGCCCCATGTGTGGAGGAACGGGCGGTTGGCCAGGCGTAAGCGGCTG
GGTTGTCTGCCGGCCCTGCAATGGCACTGGAACCCCCAAGCCCGAGGAATCGGC
GTGACGGTCGCAAACCATCCGGCCCGGTACAAATCGGCGCGGCGCTGGGTGATG
ACCTGGTGGAGAAGTTGAAGGCCGCGCAGGCCGCCCAGCGGCAACGCATCGAGG
CAGAAGCACGCCCCGGTGAATCGTGGCAAGCGGCCGCTGATCGAATCCGCAAAG
AATCCCGGCAACCGCCGGCAGCCGGTGCGCCGTCGATTAGGAAGCCGCCCAAGG
GCGACGAGCAACCAGATTTTTTCGTTCCGATGCTCTATGACGTGGGCACCCGCGA
TAGTCGCAGCATCATGGACGTGGCCGTTTTCCGTCTGTCGAAGCGTGACCGACGA
GCTGGCGAGGTGATCCGCTACGAGCTTCCAGACGGGCACGTAGAGGTTTCCGCA
GGGCCGGCCGGCATGGCCAGTGTGTGGGATTACGACCTGGTACTGATGGCGGTTT
CCCATCTAACCGAATCCATGAACCGATACCGGGAAGGGAAGGGAGACAAGCCCG
GCCGCGTGTTCCGTCCACACGTTGCGGACGTACTCAAGTTCTGCCGGCGAGCCGA
TGGCGGAAAGCAGAAAGACGACCTGGTAGAAACCTGCATTCGGTTAAACACCAC
GCACGTTGCCATGCAGCGTACGAAGAAGGCCAAGAACGGCCGCCTGGTGACGGT
ATCCGAGGGTGAAGCCTTGATTAGCCGCTACAAGATCGTAAAGAGCGAAACCGG
GCGGCCGGAGTACATCGAGATCGAGCTAGCTGATTGGATGTACCGCGAGATCAC
AGAAGGCAAGAACCCGGACGTGCTGACGGTTCACCCCGATTACTTTTTGATCGAT
CCCGGCATCGGCCGTTTTCTCTACCGCCTGGCACGCCGCGCCGCAGGCAAGGCAG
AAGCCAGATGGTTGTTCAAGACGATCTACGAACGCAGTGGCAGCGCCGGAGAGT
TCAAGAAGTTCTGTTTCACCGTGCGCAAGCTGATCGGGTCAAATGACCTGCCGGA
GTACGATTTGAAGGAGGAGGCGGGGCAGGCTGGCCCGATCCTAGTCATGCGCTA
CCGCAACCTGATCGAGGGCGAAGCATCCGCCGGTTCCTAATGTACGGAGCAGAT
GCTAGGGCAAATTGCCCTAGCAGGGGAAAAAGGTCGAAAAAGCTTCTTTCCTGT
GGATAGCACGTACATTGGGAACCCAAAGCCGTACATTGGGAACCGGAACCCGTA
CATTGGGAACCCAAAGCCGTACATTGGGAACCGGTCACACATGTAAGTGACTGA
TATAAAAGAGAAAAAAGGCGATTTTTCCGCCTAAAACTCTTTAAAACTTATTAAA
ACTCTTAAAACCCGCCTGGCCTGTGCATAACTGTCTGGCCAGCGCACAGCCGAAC
AGCTGCAAAAAGCGCCTACCCTTCGGTCGCTGCGCTCCCTACGCCCCGCCGCTTC
GCGTCGGCCTATCGCGGCCGCTGGCCGCTCAAAAATGGCTGGCCTACGGCCAGG
CAATCTACCAGGGCGCGGACAAGCCGCGCCGTCGCCACTCGACCGCCGGCGCCC
ACATCAAGGCTCCGAGTGCGCGGAACCCCTATTTGTTTATTTTTCTAAATACATTC
AAATATGTATCCGCTCATGAGACAATAACCCTGATAAATGCTTCAATAATATTGA
AAAAGGAAGAGTATGGCTAAAATGAGAATATCACCGGAATTGAAAAAACTGATC
GAAAAATACCGCTGCGTAAAAGATACGGAAGGAATGTCTCCTGCTAAGGTATAT
AAGCTGGTGGGAGAAAATGAAAACCTATATTTAAAAATGACGGACAGCCGGTAT
AAAGGGACCACCTATGATGTGGAACGGGAAAAGGACATGATGCTATGGCTGGAA
GGAAAGCTGCCTGTTCCAAAGGTCCTGCACTTTGAACGGCATGATGGCTGGAGC
AATCTGCTCATGAGTGAGGCCGATGGCGTCCTTTGCTCGGAAGAGTATGAAGATG
AACAAAGCCCTGAAAAGATTATCGAGCTGTATGCGGAGTGCATCAGGCTCTTTCA
CTCCATCGACATATCGGATTGTCCCTATACGAATAGCTTAGACAGCCGCTTAGCC
GAATTGGATTACTTACTGAATAACGATCTGGCCGATGTGGATTGCGAAAACTGGG
AAGAGGACACTCCATTTAAAGATCCGCGCGAGCTGTATGATTTTTTAAAGACGGA
AAAGCCCGAAGAGGAACTTGTCTTTTCCCACGGCGACCTGGGAGACAGCAACAT
CTTTGTGAAAGATGGCAAAGTAAGTGGCTTTATTGATCTTGGGAGAAGCGGCAG
GGCGGACAAGTGGTATGACATTGCCTTCTGCGTCCGGTCGCTCAGGGAGGATATC
GGGGAAGAACAGTATGTCGAGCTATTTTTTGACTTACTGGGGATCAAGCCTGATT
GGGAGAAAATAAAATATTATATTTTACTGGATGAATTGTTTTAGCTGTCAGACCA
AGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGC
TTCAGCAGAGCGCAGATACCAAATACTGTTCTTCTAGTGTAGCCGTAGTTAGGCC
ACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCGGCCT
TTTTACGGTTCCTGCTCGGATCTGTTGGACCGGACAGTAGTCATGGTTGATGGGC
TGCCTGTATCGAGTGGTGATTTTGTGCCGAGCTGCCGGTCGGGGAGCTGTTGGCT
GGCTGGTGGCAGGATATATTGTGGTGTAAACAAATTGACGCTTAGACAACTTAAT
AACACATTGCGGACGTTTTTAATGTACTGGGGTTGAACACTCT

The final binary vectors were introduced into the tomato cultivar M82 by Agrobacterium tumefaciens-mediated transformation (Gupta and Van Eck, 2016). Recovered transgenic plants were transplanted on soil and allowed to grow on long days (16 hours light/8 hours dark) in a greenhouse supplemented with an artificial light source from high-pressure sodium bulbs ˜250 μmol m⁻² s⁻¹). These first-generation (T0) transgenic plants were then genotyped for CRISPR/Cas9-mediated lesions by extracting DNA from main and axillary shoots and carrying out PCR to amplify the target region upstream of the TSS of SlCLV3, using primers binding between 250 and 400 bp away from each of the outermost sgRNAs. PCR products were analyzed by gel electrophoresis, and products were cloned into pSC-A-amp/kan vector (Agilent) following manufacturer's instructions. At least 3 clones per sample were sequenced using 6 primers spanning the target region.

### A Reverse/Forward Genetic Screen to Generate New Transcriptional Alleles in SlCLV3

A sensitized first generation outcross (F1) was produced, comprising a population of seeds being heterozygous for a knockout allele of SlCLV3 and hemizygous for the CRISPR/Cas9 transgene targeting the 2000 bp upstream region of SlCLV3, by emasculating and hand-pollinating several dozens of flowers from the reference cultivar M82 with the transgenic line T0-2. Ripened fruits were harvested and seeds were extracted manually, treated for 1 hour with rapidase (Centerchem), washed using a 1:3 (v:v) dilution of bleach for 10 minutes, soaked in water and then left for overnight drying in paper towels. F1 seeds were subsequently sown in 96-cell flats filled with soil mix and kept under greenhouse conditions. After germination, DNA was extracted from cotyledons and genotyping was performed to detect the CRISPR/Cas9 transgene in each individual using primers spanning the last 100 bp of the 35S promoter and the first 300 bp of the Cas9 coding sequence. Every individual carrying a copy of the transgene was transplanted in the field at the Uplands Farm of Cold Spring Harbor Laboratory. Plants were grown under drip irrigation and following standard fertilizer application regimes. Every single plant was labeled accordingly to its phenotype by a visual inspection on changes in sepal/petal number in the first inflorescences and clustered into three main categories named “weak”, “moderate”, and “strong.” Plants that did not show any visible phenotype or those with multiple phenotypic sectors were taken out to allow better growth of the other, stable phenotypic classes. Subsequently, fruit locule number was quantified from several fruits for each plant from the different phenotypic classes. DNA was extracted from moderate and strong classes and genotyped to confirm the presence of new alleles for the target region by PCR, using the same primer pairs as for the original T0 individuals.

Segregation and Characterization of New Alleles Derived from the Reverse/Forward Genetic Screen

New alleles derived from the genetic screen were segregated from progeny derived from the F1 plants and the phenotypic effect of each allele was assessed in non-transgenic (i.e., not containing the CRISPR/Cas9 construct), individuals that are homozygous for the new mutated promoter allele. Seeds were collected for every single plant of each category but only F2 moderate and strong populations were sown under greenhouse conditions. Genotyping for the presence of the transgene in the F2 populations was performed as for F1 plants. Non-transgenic individuals were kept and genotyped to determine the inheritance of the new alleles observed in the F1 parental plants by amplifying the upstream region of SlCLV3 as described. Two to six non-transgenic plants per family, carrying at least one new allele in a heterozygous state were selected and grown under greenhouse conditions. A visual inspection of floral organ number was performed and each family was classified into the same three categories as done for F1 parentals. Subsequently, DNA was extracted and PCR-based genotyping was performed to corroborate the inheritance of the alleles observed in the parental F1 population. Representative F3 families carrying homozygous individuals for a new allele, and covering the three phenotypic categories, were selected for allele characterization by sequencing of cloned PCR products using the same primers spanning the target region. The effect of the new allele in each F3 family was determined by quantifying floral organ number on several flowers from at least three individuals per family.

### Results and Conclusions

Crop improvement refers to the process whereby humans have selected both qualitative and quantitative characteristics in domesticated crops, such as flowering time, pathogen resistance, shoot architecture and fruit size, with aims to increase yield. However, crop improvement relies on the availability of genetic variation, which tends to be reduced on existing crops. Breeding programs often take advantage of standing genetic variation from cultivars, along with the introgression of new alleles from exotic germplasm coming from wild relatives. This usually leads to a complex and time-consuming breeding process to eliminate undesired genetic effects. Previous efforts have been made to introduce new genetic variation through chemical, radiation and transposon mutagenesis, and although valuable, it still requires complex efforts in order to map the causative mutations. With the advent of genomics, further insights have been made into the molecular footprint of domestication and breeding as well as the developmental processes controlling yield traits. Previous studies indicated close to half of causative mutations in domestication and quantitative trait variation are associated with gene expression changes, produced by mutations in regulatory regions. As described herein, technologies for gene editing such as CRISPR/Cas9 offer the potential for precise targeting of regulatory regions of genes involved in both qualitative and quantitative trait variation in plants, and even for the generation of new sources of genetic, and hence phenotypic variation, allowing the advancement towards a more directed approach for crop improvement.

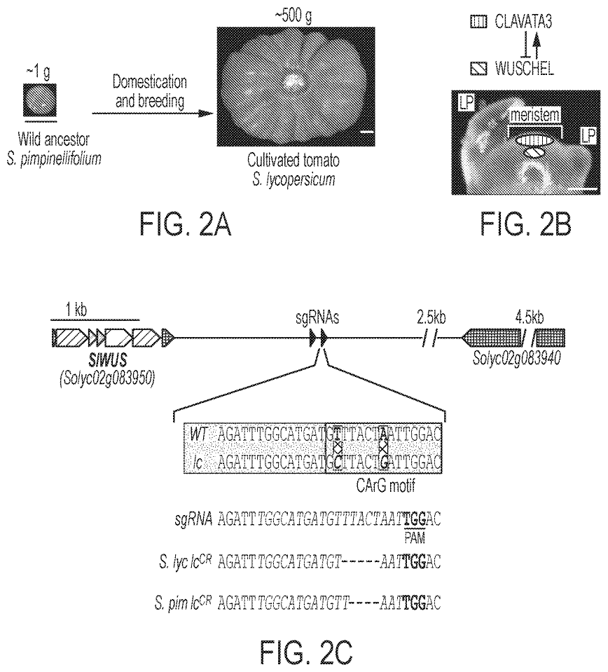

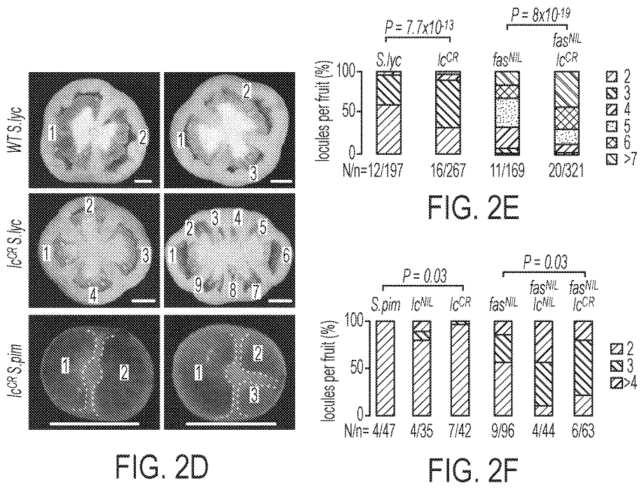

Tomato (Solanum lycopersicum L.) is one of the most cultivated crops worldwide and fruit size has been one of the main drivers of domestication and breeding in this crop (FIG. 2A). The number of stem cells in apical meristems regulates fruit size. Extensive research in several plant systems has provided evidence for a genetic circuit in which the stem cell regulators WUSCHEL (WUS) and CLAVATA3 (CLV3) are involved in regulation of the apical meristem (FIG. 2B). Alterations in the functions of these two genes lead to changes in inflorescence architecture and fruit size (Bommert et al 2013 and Je at al 2016).

In order to demonstrate the power of gene editing for generating new sources of phenotypic variation by altering gene regulatory regions, a previously described Quantitative Trait Locus (QTL) influencing fruit size known as locule number (lc) was targeted using CRISPR/Cas9. lc influences fruit size by increasing the number of locules, which are the seed bearing tissues in the developing fruit, and was previously narrowed down to a 1,080 base pairs (bp) region downstream of the tomato homolog of WUS (S/WUS). Two causative single-nucleotide polymorphisms (SNPs) in lc are associated with a disruption of a putative AGAMOUS binding site (FIG. 2C) that is also conserved in Arabidopsis thaliana. This motif was targeted using two single guide-RNAs (sgRNAs) and transgenic lines were recovered for both the wild species S. pimpinellifolium (S. pimp) and the domesticated tomato reference cultivar M82, both which lack the lc allele. Disruption of the motif caused a weak increase in locule number in both backgrounds, shifting the frequency from two to three locules per fruit in S. pimp and from two to four or more in M82 (FIG. 2D). Previous studies indicated that during tomato domestication, the close association of lc and another QTL with stronger effect on locule number known as fasciated (fas) led to the current diversity of fruit size in cultivated tomato. Remarkably, fas was recently shown to be a regulatory mutation in CLAVATA3 (SlCLV3). A synergistic interaction between these two QTLs led to increased locule number, hence increasing fruit size. The CRISPR-lc (lc^{CR}) transgenics were crossed to fas near-isogenic lines (fas^{NIL}) of both S. pimp and M82, and nontransgenic double homozygous mutants were recovered. Notably, double mutants showed enhancement for increased locule number, thus confirming the interaction between lc and fas and providing genetic support for the previously described lc QTL (FIGS. 2E and 2F).

Several previous QTLs were described as affecting fruit size in tomato, with fas exerting a major effect. As a consequence of the role of SlCLV3 in fruit size change in tomato during domestication and breeding, only the single variant fas has been found among cultivars and landraces. However, recent CRISPR/Cas9 targeting of the SlCLV3 gene coding sequence in tomato (clv3^{CR}) showed that fas is an allele with moderate effect on locule number. These previous studies show that phenotypic variation is possible but do not provide a method for producing a variety of phenotypes quickly and efficiently.

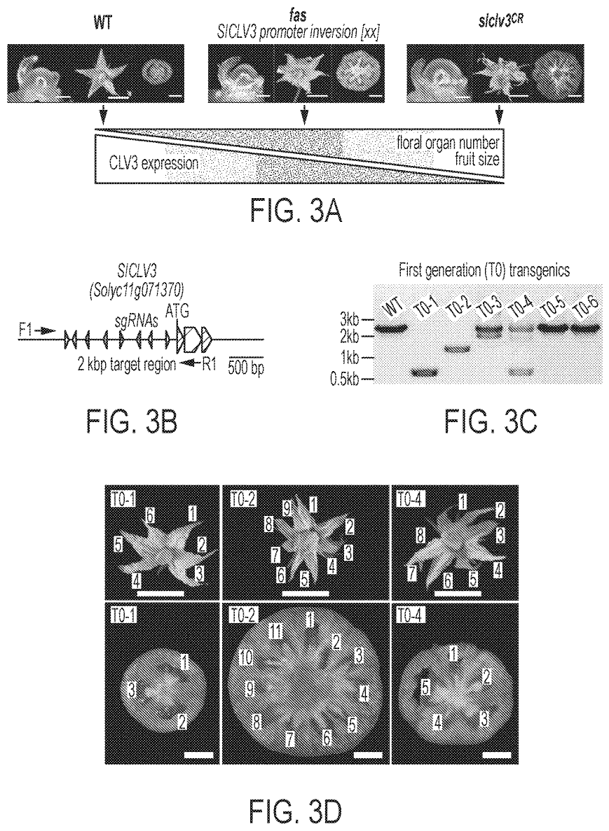

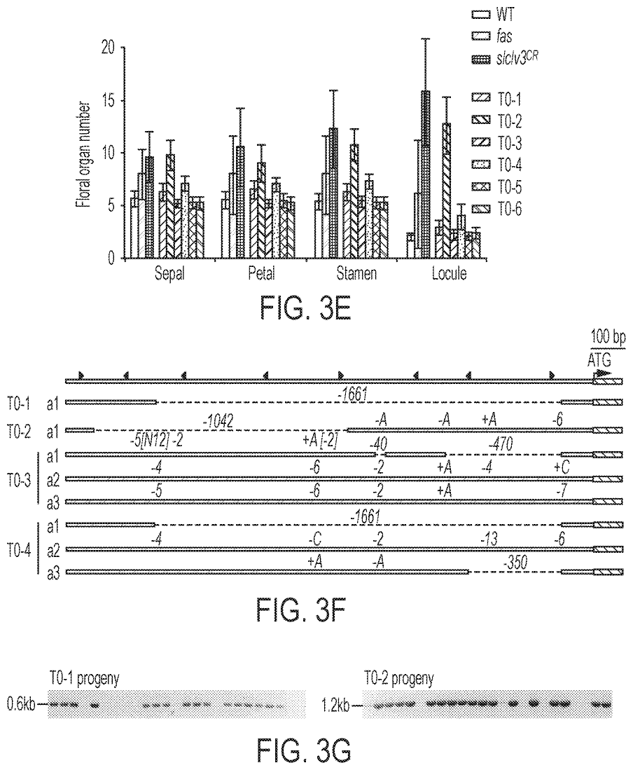

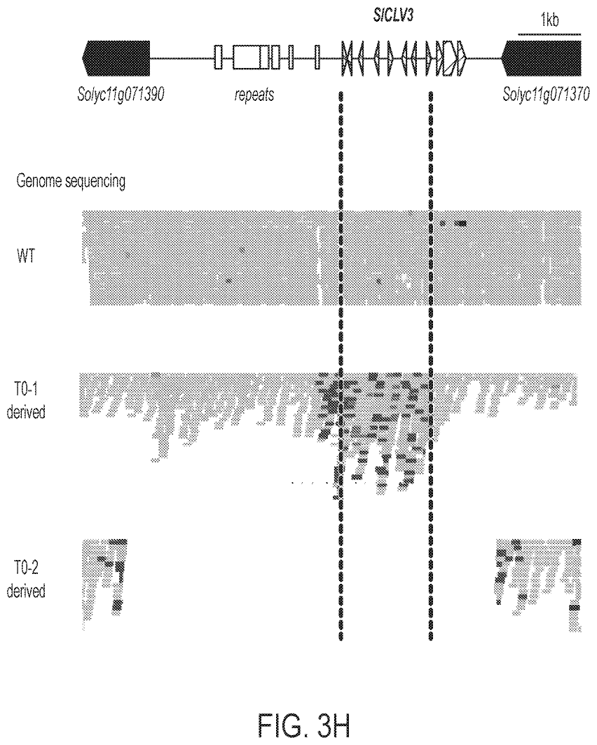

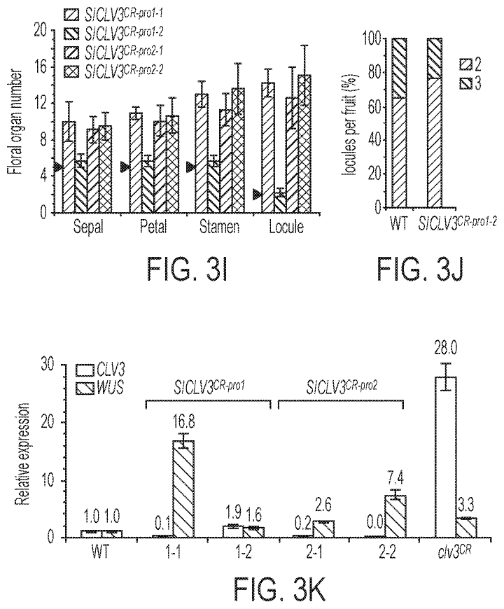

It was hypothesized herein that it would be possible to engineer quantitative phenotypic variation on locule number, and hence fruit size, by targeting the regulatory regions of SlCLV3, thus modifying its transcriptional expression (FIG. 3A). To achieve this, a CRISPR/Cas9 construct was generated with an array of eight sgRNAs targeting 2 kilobases (kb) upstream of the transcriptional start site in SlCLV3. Each sgRNA was spaced between 200-400 bp apart from each other sgRNA, with no special bias for targeting any known regulatory motifs (FIG. 3B). The six first generation transgenic lines (T0) were recovered and the region upstream of the transcriptional start site in SlCLV3 was screened by PCR, looking primarily for large deletions caused by some combination of the activity of the eight sgRNAs. A considerable range of deletion sizes was clearly visible by PCR (FIG. 3C), indicating the activity of the eight sgRNAs led to a diverse range of alleles and not simply the entire deletion of the target region. Notably, a range of weak to strong phenotypic effects was also observed, visible on flower organs and as a fruit size increase among T0 lines (FIG. 3D). When compared to M82, fas and slclv3^{CR}, four of the T0 lines showed quantitative differences (FIG. 3E), implying the new alleles generated by CRISPR/Cas9 were able to produce a range of new phenotypic variation.

Four of the T0 lines that showed significant phenotypic differences were sequenced and several different alleles were identified, ranging from an entire deletion of the target region to small deletions and insertions of one to thirteen bp in size (FIG. 3F). Interestingly, two of the original T0 lines appeared homozygous for large deletion alleles. To confirm its genetic constitution and the heritability of the alleles into the next generation, the T1 progeny was analyzed and it was found that both T0-1 and T0-2 were actually biallelic plants, each carrying a PCR-visible allele and a non-amplifiable allele (FIGS. 3F and 3G). Genomic sequencing of progeny from these two lines confirmed the presence of what appeared to be a duplication of the target region in T0-1 and a massive 7.3 kb deletion from T0-2, in which even the SlCLV3 coding sequence was completely deleted (FIG. 3H). Next, the floral organ number was analyzed by dissecting flowers and counting the number of sepals, petals, stamens and locules in homozygous plants for the four new alleles generated using the CRISPR/Cas9 construct. Quantitative differences were found between the plants, particularly for locule number (FIGS. 31 and 3J). Remarkably, the T0-1 duplication-derived allele showed significant reduction in locule number compared to M82, indicating that this allele might actually be a gain-of-function version of SlCLV3. qRT-PCR expression analysis on apical meristem close to reproductive transition showed quantitative changes of SlCLV3 expression in T0-1 and T0-2 derived alleles, confirming the quantitative transcriptional effect of targeting regulatory regions (FIG. 3K)

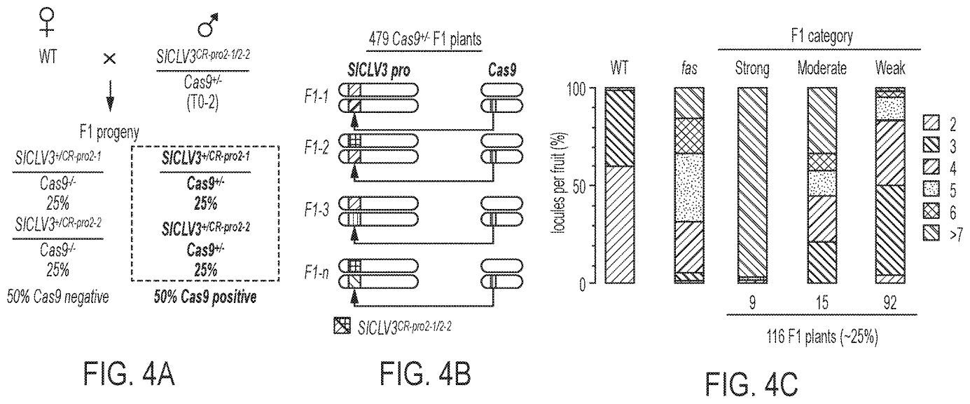

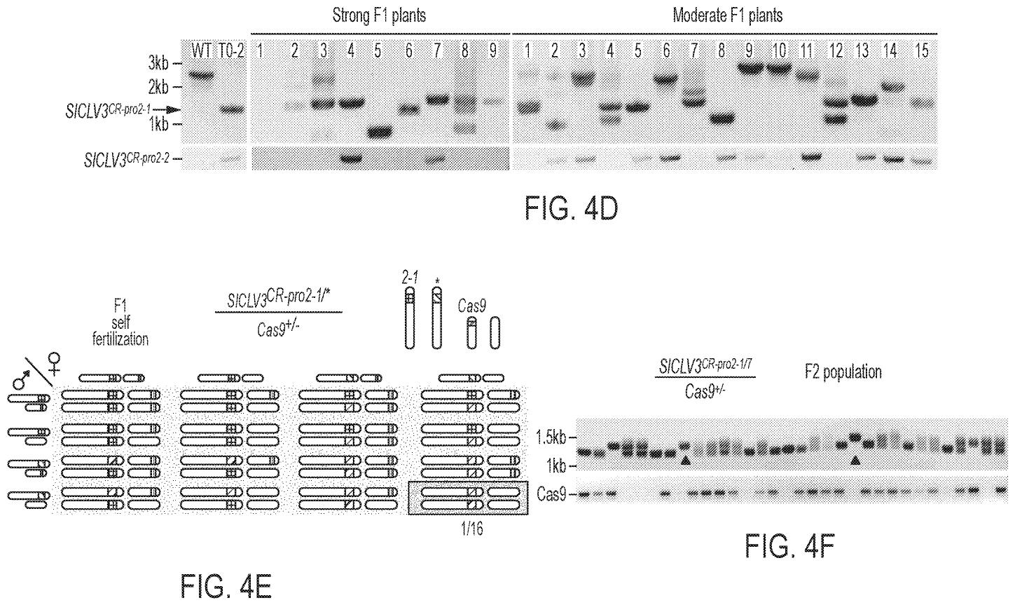

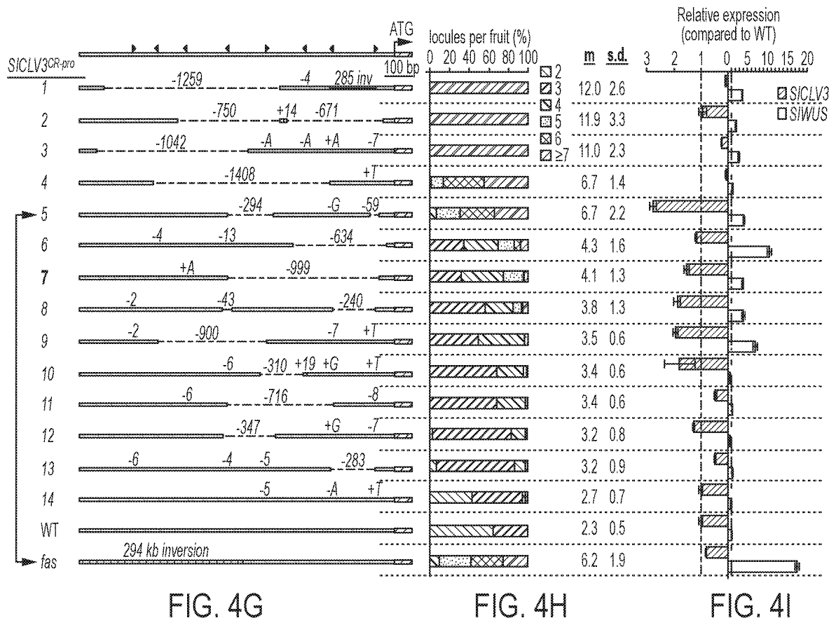

To maximize the potential for generating new alleles with quantitative effects by CRISPR/Cas9 targeting, one of the biallelic T0 lines (T0-2) with high locule number phenotype was used to outcross with wild-type M82 plants and set up a reverse/forward genetic screen (FIG. 4A). Briefly, F1 progeny were hemizygous for the CRISPR/Cas9 transgene, carried one of the two alleles from T0-2 and a wild-type allele from M82. More specifically, 479 (˜50%) F1 hemizygous transgenic plants were obtained from a total population of about 1200. In these plants, the CRISPR/Cas9 transgene was hypothesized to target the wild-type allele present, generating a new mutant allele in the sensitized background of the biallelic T0 line having a relatively strong phenotype, allowing for easier screening and identification of phenotypic effects of the newly generated allele (FIG. 4B). Consistently, a visual screen of the plants growing under field conditions showed that 116 out of the 479 (˜25%) exhibited increasing floral organ numbers. The phenotypes were clustered into three categories (weak, moderate and strong). Ultimately, the locule number on fruits from 114 plants from these categories were quantified and quantitative differences in the three categories were found when compared to wild type and the reference allele fas (FIG. 4C). PCR analysis in the moderate and strong categories confirmed the presence of new alleles in each individual plant, along with the expected segregation of the T0-2 derived alleles (FIG. 4D), indicating the successful CRISPR/Cas9-mediated de novo generation of new alleles.

To properly assess the inheritance and phenotypic effect of new alleles coming from the screen, nontransgenic (i.e., absence of CRISPR/Cas9 transgene) homozygous individuals in the F2 generation were analyzed (FIG. 4E). Fourteen F2 families were selected representing the above-mentioned phenotypic classes and covering a range of PCR-based different-sized alleles, named as SlCLV3pro^{CR}. Individual plants from these families were analyzed by Sanger sequencing and the floral organ number in ˜100 flowers per allele were quantified (FIG. 4F). A high diversity of alleles were observed after sequencing and assembly, indicating that CRISPR/Cas9 targeting using multiple sgRNAs is effective in producing a diverse set of mutant alleles in large target regions (FIG. 4G). Remarkably, the fourteen alleles covered a vast array of quantitative effects in locule number, ranging from almost wild type to resembling the clv3^{CR} allele (FIG. 4H), indicating that it is feasible to manipulate locule number quantitatively by targeting regulatory regions of SlCLV3. To confirm that the quantitative locule number change caused by each allele was due to SlCLV3 expression changes, qRT-PCR expression analysis was carried out for SlCLV3 in the 14 alleles and the expression levels were compared to wild type, fas and clv3′ (FIG. 4I).

These data confirm that targeting regulatory regions can result in quantitative trait variation, highlighting the previous known role of regulatory alleles on domestication and breeding. The strategy herein utilized gene editing using CRISPR/Cas9 technology to produce genetic variation that changes the expression —hence the activity—of a single gene in a controlled and directed manner. Additionally, this gene editing strategy provides new molecular and genetic sources for studying the role of regulatory regions and mechanisms controlling gene expression, both at the level of cis and trans regulation, including the effects of chromatin and epigenetics involved in stem cell homeostasis in tomato. This strategy could be harnessed to optimize breeding programs by targeting specific sets of genes with major effects, taking advantage of genomic information regarding the developmental patterns and genes controlling yield traits, alleviating in part the drawback of dealing with time-consuming QTL stacking and complex epistatic effects.

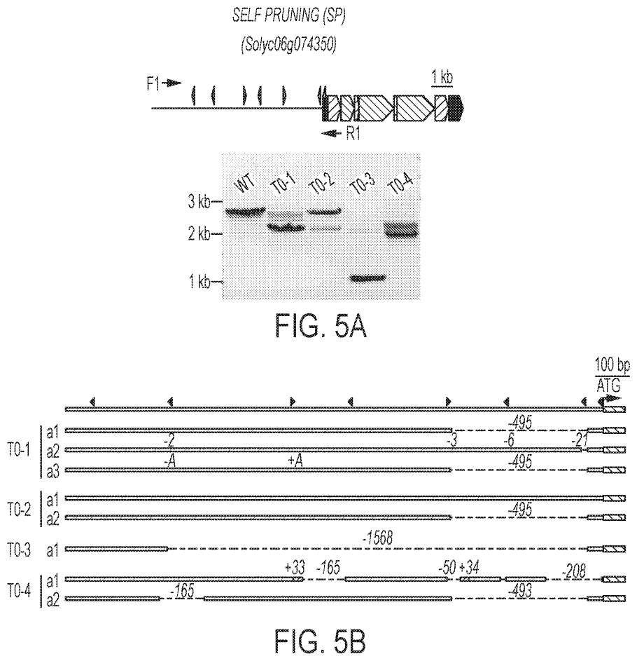

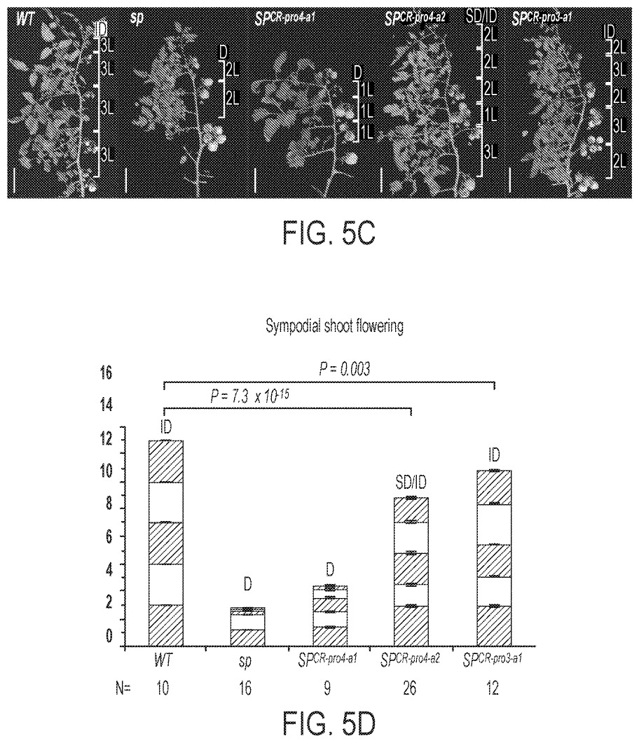

This gene editing approach may be generally applied to other yield traits, such as inflorescence architecture, pathogen resistance, flowering time and others, not only in tomato, but also in other major crops. For instance, the SELF PRUNING (SP) gene is involved in controlling shoot determinacy in tomato, with null mutants showing determinate growth. A similar strategy of CRISPR/Cas9 regulatory sequence targeting was undertaken and several alleles were recovered in T0 plants, and analyzed in stable nontransgenic T2 progeny (FIGS. 5A and B). A quantitative change was observed for sympodial shoot index by characterizing 3 new alleles generated (FIGS. 5C and 4D), strongly supporting that this strategy provides a powerful tool to engineer new quantitative trait variation for crop improvement.

### Annotated SP CRISPR/Cas9 Construct

CaMV 2x35s promoter: 1904-2656 bp
Cas9: 2743-6883 bp
sgRNA1 guide sequence: 7250-7269 bp
sgRNA1 scaffold sequence: 7270-7345 bp
sgRNA2 guide sequence: 7486-7505 bp
sgRNA2 scaffold sequence: 7506-7581 bp
sgRNA3 guide sequence: 7722-7741 bp
sgRNA3 scaffold sequence: 7742-7817 bp
sgRNA4 guide sequence: 7958-7977 bp
sgRNA4 scaffold sequence: 7978-8053 bp
sgRNA5 guide sequence: 8194-8213 bp
sgRNA5 scaffold sequence: 8214-8289 bp
sgRNA6 guide sequence: 8431-8450 bp
sgRNA6 scaffold sequence: 8451-8526 bp
sgRNA7 guide sequence: 8667-8686 bp
sgRNA7 scaffold sequence: 8687-8762 bp
sgRNA8 guide sequence: 8903-8922 bp
sgRNA8 scaffold sequence: 8923-8998 bp
(SEQ ID NO: 3)
GTGCCGAATTCGGATCCGGAGCGGAGAATTAAGGGAGTCACGTTATGACCCCCG
CCGATGACGCGGGACAAGCCGTTTTACGTTTGGAACTGACAGAACCGCAACGAT
TGAAGGAGCCACTCAGCCGCGGGTTTCTGGAGTTTAATGAGCTAAGCACATACGT
CAGAAACCATTATTGCGCGTTCAAAAGTCGCCTAAGGTCACTATCAGCTAGCAAA
TATTTCTTGTCAAAAATGCTCCACTGACGTTCCATAAATTCCCCTCGGTATCCAAT
TAGAGTCTCATATTCACTCGACTTTTACAACAATTACCAACAACAACAAACAACA
AACAACATTACAATTACTATTTACAATTATCCATGGTTGAACAAGATGGATTGCA
CGCAGGTTCTCCGGCCGCTTGGGTGGAGAGGCTATTCGGCTATGACTGGGCACAA
CAGACAATCGGCTGCTCTGATGCCGCCGTGTTCCGGCTGTCAGCGCAGGGGCGCC
CGGTTCTTTTTGTCAAGACCGACCTGTCCGGTGCCCTGAATGAACTGCAGGACGA
GGCAGCGCGGCTATCGTGGCTGGCCACGACGGGCGTTCCTTGCGCAGCTGTGCTC
GACGTTGTCACTGAAGCGGGAAGGGACTGGCTGCTATTGGGCGAAGTGCCGGGG
CAGGATCTCCTGTCATCTCACCTTGCTCCTGCCGAGAAAGTATCCATCATGGCTG
ATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGC
CAGGCTCAAGGCGCGCATGCCCGACGGCGAGGATCTCGTCGTGACTCATGGCGA
TGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTG
ATATTGCTGAAGAGCTTGGCGGCGAATGGGCTGACCGCTTCCTCGTGCTTTACGG
TATCGCCGCTCCCGATTCGCAGCGCATCGCCTTCTATCGCCTTCTTGACGAGTTCT
TCTGAGCGGGACTCTGGGGTTCGCTGCTTTAATGAGATATGCGAGACGCCTATGA
TCGCATGATATTTGCTTTCAATTCTGTTGTGCACGTTGTAAAAAACCTGAGCATGT
GTAGCTCAGATCCTTACCGCCGGTTTCGGTTCATTCTAATGAATATATCACCCGTT
ACTATCGTATTTTTATGAATAATATTCTCCGTTCAATTTACTGATTGTACCCTACT
ACTTATATGTACAATATTAAAATGAAAACAATATATTGTGCTGAATAGGTTTATA
GCGACATCTATGATAGAGCGCCACAATAACAAACAATTGCGTTTTATTATTACAA
ATCCAATTTTAAAAAAAGCGGCAGAACCGGTCAAACCTAAAAGACTGATTACAT
AAATCTTATTCAAATTTCAAAAGGCCCCAGGGGCTAGTATCTACGACACACCGAG
CGGCGAACTAATAACGTTCACTGAAGGGAACTCCGGTTCCCCGCCGGCGCGCAT
GGGTGAGATTCCTTGAAGTTGAGTATTGGCCGTCCGCTCTACCGAAAGTTACGGG
CACCATTCAACCCGGTCCAGCACGGCGGCCGGGTAACCGACTTGCTGCCCCGAG
AATTATGCAGCATTTTTTTGGTGTATGTGGGCCCCAAATGAAGTGCAGGTCAAAC
CTTGACAGTGACGACAAATCGTTGGGCGGGTCCAGGGCGAATTTTGCGACAACA
TGTCGAGGCTCAGCCGCTGCAAGAATTCAAGCTTGGAGGTCAACATGGTGGAGC
ACGACACTCTGGTCTACTCCAAAAATGTCAAAGATACAGTCTCAGAAGATCAAA
GGGCTATTGAGACTTTTCAACAAAGGATAATTTCGGGAAACCTCCTCGGATTCCA
TTGCCCAGCTATCTGTCACTTCATCGAAAGGACAGTAGAAAAGGAAGGTGGCTC
CTACAAATGCCATCATTGCGATAAAGGAAAGGCTATCATTCAAGATCTCTCTGCC
GACAGTGGTCCCAAAGATGGACCCCCACCCACGAGGAGCATCGTGGAAAAAGAA
GAGGTTCCAACCACGTCTACAAAGCAAGTGGATTGATGTGATAACATGGTGGAG
CACGACACTCTGGTCTACTCCAAAAATGTCAAAGATACAGTCTCAGAAGATCAA
AGGGCTATTGAGACTTTTCAACAAAGGATAATTTCGGGAAACCTCCTCGGATTCC
ATTGCCCAGCTATCTGTCACTTCATCGAAAGGACAGTAGAAAAGGAAGGTGGCT
CCTACAAATGCCATCATTGCGATAAAGGAAAGGCTATCATTCAAGATCTCTCTGC
CGACAGTGGTCCCAAAGATGGACCCCCACCCACGAGGAGCATCGTGGAAAAAGA
AGAGGTTCCAACCACGTCTACAAAGCAAGTGGATTGATGTGACATCTCCACTGAC
GTAAGGGATGACGCACAATCCCACTATCCTTCGCAAGACCCTTCCTCTATATAAG
GAAGTTCATTTCATTTGGAGAGGACACGCTCGAGTATAAGAGCTCATTTTTACAA
CAATTACCAACAACAACAAACAACAAACAACATTACAATTACATTTACAATTATC
GATACAATGGACAAGAAGTACTCCATTGGGCTCGATATCGGCACAAACAGCGTC
GGCTGGGCCGTCATTACGGACGAGTACAAGGTGCCGAGCAAAAAATTCAAAGTT
CTGGGCAATACCGATCGCCACAGCATAAAGAAGAACCTCATTGGCGCCCTCCTGT
TCGACTCCGGGGAGACGGCCGAAGCCACGCGGCTCAAAAGAACAGCACGGCGC
AGATATACCCGCAGAAAGAATCGGATCTGCTACCTGCAGGAGATCTTTAGTAAT
GAGATGGCTAAGGTGGATGACTCTTTCTTCCATAGGCTGGAGGAGTCCTTTTTGG
TGGAGGAGGATAAAAAGCACGAGCGCCACCCAATCTTTGGCAATATCGTGGACG
AGGTGGCGTACCATGAAAAGTACCCAACCATATATCATCTGAGGAAGAAGCTTG
TAGACAGTACTGATAAGGCTGACTTGCGGTTGATCTATCTCGCGCTGGCGCATAT
GATCAAATTTCGGGGACACTTCCTCATCGAGGGGGACCTGAACCCAGACAACAG
CGATGTCGACAAACTCTTTATCCAACTGGTTCAGACTTACAATCAGCTTTTCGAA
GAGAACCCGATCAACGCATCCGGAGTTGACGCCAAAGCAATCCTGAGCGCTAGG
CTGTCCAAATCCCGGCGGCTCGAAAACCTCATCGCACAGCTCCCTGGGGAGAAG
AAGAACGGCCTGTTTGGTAATCTTATCGCCCTGTCACTCGGGCTGACCCCCAACT
TTAAATCTAACTTCGACCTGGCCGAAGATGCCAAGCTTCAACTGAGCAAAGACA
CCTACGATGATGATCTCGACAATCTGCTGGCCCAGATCGGCGACCAGTACGCAG
ACCTTTTTTTGGCGGCAAAGAACCTGTCAGACGCCATTCTGCTGAGTGATATTCT
GCGAGTGAACACGGAGATCACCAAAGCTCCGCTGAGCGCTAGTATGATCAAGCG
CTATGATGAGCACCACCAAGACTTGACTTTGCTGAAGGCCCTTGTCAGACAGCAA
CTGCCTGAGAAGTACAAGGAAATTTTCTTCGATCAGTCTAAAAATGGCTACGCCG
GATACATTGACGGCGGAGCAAGCCAGGAGGAATTTTACAAATTTATTAAGCCCA
TCTTGGAAAAAATGGACGGCACCGAGGAGCTGCTGGTAAAGCTTAACAGAGAAG
ATCTGTTGCGCAAACAGCGCACTTTCGACAATGGAAGCATCCCCCACCAGATTCA
CCTGGGCGAACTGCACGCTATCCTCAGGCGGCAAGAGGATTTCTACCCCTTTTTG
AAAGATAACAGGGAAAAGATTGAGAAAATCCTCACATTTCGGATACCCTACTAT
GTAGGCCCCCTCGCCCGGGGAAATTCCAGATTCGCGTGGATGACTCGCAAATCA
GAAGAGACTATCACTCCCTGGAACTTCGAGGAAGTCGTGGATAAGGGGGCCTCT
GCCCAGTCCTTCATCGAAAGGATGACTAACTTTGATAAAAATCTGCCTAACGAAA
AGGTGCTTCCTAAACACTCTCTGCTGTACGAGTACTTCACAGTTTATAACGAGCT
CACCAAGGTCAAATACGTCACAGAAGGGATGAGAAAGCCAGCATTCCTGTCTGG
AGAGCAGAAGAAAGCTATCGTGGACCTCCTCTTCAAGACGAACCGGAAAGTTAC
CGTGAAACAGCTCAAAGAAGATTATTTCAAAAAGATTGAATGTTTCGACTCTGTT
GAAATCAGCGGAGTGGAGGATCGCTTCAACGCATCCCTGGGAACGTATCACGAT
CTCCTGAAAATCATTAAAGACAAGGACTTCCTGGACAATGAGGAGAACGAGGAC
ATTCTTGAGGACATTGTCCTCACCCTTACGTTGTTTGAAGATAGGGAGATGATTG
AAGAACGCTTGAAAACTTACGCTCATCTCTTCGACGACAAAGTCATGAAACAGCT
CAAGAGGCGCCGATATACAGGATGGGGGCGGCTGTCAAGAAAACTGATCAATGG
GATCCGAGACAAGCAGAGTGGAAAGACAATCCTGGATTTTCTTAAGTCCGATGG
ATTTGCCAACCGGAACTTCATGCAGTTGATCCATGATGACTCTCTCACCTTTAAG
GAGGACATCCAGAAAGCACAAGTTTCTGGCCAGGGGGACAGTCTCCACGAGCAC
ATCGCTAATCTTGCAGGTAGCCCAGCTATCAAAAAGGGAATACTGCAGACCGTT
AAGGTCGTGGATGAACTCGTCAAAGTAATGGGAAGGCATAAGCCCGAGAATATC
GTTATCGAGATGGCCCGAGAGAACCAAACTACCCAGAAGGGACAGAAGAACAG
TAGGGAAAGGATGAAGAGGATTGAAGAGGGTATAAAAGAACTGGGGTCCCAAA
TCCTTAAGGAACACCCAGTTGAAAACACCCAGCTTCAGAATGAGAAGCTCTACCT
GTACTACCTGCAGAACGGCAGGGACATGTACGTGGATCAGGAACTGGACATCAA
TCGGCTCTCCGACTACGACGTGGATCATATCGTGCCCCAGTCTTTTCTCAAAGAT
GATTCTATTGATAATAAAGTGTTGACAAGATCCGATAAAAATAGAGGGAAGAGT
GATAACGTCCCCTCAGAAGAAGTTGTCAAGAAAATGAAAAATTATTGGCGGCAG
CTGCTGAACGCCAAACTGATCACACAACGGAAGTTCGATAATCTGACTAAGGCT
GAACGAGGTGGCCTGTCTGAGTTGGATAAAGCCGGCTTCATCAAAAGGCAGCTT
GTTGAGACACGCCAGATCACCAAGCACGTGGCCCAAATTCTCGATTCACGCATG
AACACCAAGTACGATGAAAATGACAAACTGATTCGAGAGGTGAAAGTTATTACT
CTGAAGTCTAAGCTGGTTTCAGATTTCAGAAAGGACTTTCAGTTTTATAAGGTGA
GAGAGATCAACAATTACCACCATGCGCATGATGCCTACCTGAATGCAGTGGTAG
GCACTGCACTTATCAAAAAATATCCCAAGCTTGAATCTGAATTTGTTTACGGAGA
CTATAAAGTGTACGATGTTAGGAAAATGATCGCAAAGTCTGAGCAGGAAATAGG
CAAGGCCACCGCTAAGTACTTCTTTTACAGCAATATTATGAATTTTTTCAAGACC
GAGATTACACTGGCCAATGGAGAGATTCGGAAGCGACCACTTATCGAAACAAAC
GGAGAAACAGGAGAAATCGTGTGGGACAAGGGTAGGGATTTCGCGACAGTCCG
GAAGGTCCTGTCCATGCCGCAGGTGAACATCGTTAAAAAGACCGAAGTACAGAC
CGGAGGCTTCTCCAAGGAAAGTATCCTCCCGAAAAGGAACAGCGACAAGCTGAT
CGCACGCAAAAAAGATTGGGACCCCAAGAAATACGGCGGATTCGATTCTCCTAC
AGTCGCTTACAGTGTACTGGTTGTGGCCAAAGTGGAGAAAGGGAAGTCTAAAAA
ACTCAAAAGCGTCAAGGAACTGCTGGGCATCACAATCATGGAGCGATCAAGCTT
CGAAAAAAACCCCATCGACTTTCTCGAGGCGAAAGGATATAAAGAGGTCAAAAA
AGACCTCATCATTAAGCTTCCCAAGTACTCTCTCTTTGAGCTTGAAAACGGCCGG
AAACGAATGCTCGCTAGTGCGGGCGAGCTGCAGAAAGGTAACGAGCTGGCACTG
CCCTCTAAATACGTTAATTTCTTGTATCTGGCCAGCCACTATGAAAAGCTCAAAG
GATCTCCCGAAGATAATGAGCAGAAGCAGCTGTTCGTGGAACAACACAAACACT
ACCTTGATGAGATCATCGAGCAAATAAGCGAATTCTCCAAAAGAGTGATCCTCG
CCGACGCTAACCTCGATAAGGTGCTTTCTGCTTACAATAAGCACAGGGATAAGCC
CATCAGGGAGCAGGCAGAAAACATTATCCACTTGTTTACTCTGACCAACTTGGGC
GCGCCTGCAGCCTTCAAGTACTTCGACACCACCATAGACAGAAAGCGGTACACC
TCTACAAAGGAGGTCCTGGACGCCACACTGATTCATCAGTCAATTACGGGGCTCT
ATGAAACAAGAATCGACCTCTCTCAGCTCGGTGGAGACAGCAGGGCTGACCCCA
AGAAGAAGAGGAAGGTGTGAGCTTGTCAAGCAGATCGTTCAAACATTTGGCAAT
AAAGTTTCTTAAGATTGAATCCTGTTGCCGGTCTTGCGATGATTATCATATAATTT
CTGTTGAATTACGTTAAGCATGTAATAATTAACATGTAATGCATGACGTTATTTA
TGAGATGGGTTTTTATGATTAGAGTCCCGCAATTATACATTTAATACGCGATAGA
AAACAAAATATAGCGCGCAAACTAGGATAAATTATCGCGCGCGGTGTCATCTAT
GTTACTAGATCGACGCTACTAGAATTCGAGCTCGGAGTGATCAAAAGTCCCACAT
CGATCAGGTGATATATAGCAGCTTAGTTTATATAATGATAGAGTCGACATAGCGA
TTAAAGAGTTGTAGTTGTTTTTGTTTTAGAGCTAGAAATAGCAAGTTAAAATAAG
GCTAGTCCGTTATCAACTTGAAAAAGTGGCACCGAGTCGGTGCTTTTTTTCTAGA
CCCAGCTTTCTTGTACAAAGTTGGCATTACGCTTTACGAATTCCCATGGGGAGTG
ATCAAAAGTCCCACATCGATCAGGTGATATATAGCAGCTTAGTTTATATAATGAT
AGAGTCGACATAGCGATTAGATCATTAGAGAGTCAGATGTTTTAGAGCTAGAAA
TAGCAAGTTAAAATAAGGCTAGTCCGTTATCAACTTGAAAAAGTGGCACCGAGT
CGGTGCTTTTTTTCTAGACCCAGCTTTCTTGTACAAAGTTGGCATTACGCTCAGAG
AATTCGCATGCGGAGTGATCAAAAGTCCCACATCGATCAGGTGATATATAGCAG
CTTAGTTTATATAATGATAGAGTCGACATAGCGATTGAAAGGTGAGAGCTTGTTG
TGTTTTAGAGCTAGAAATAGCAAGTTAAAATAAGGCTAGTCCGTTATCAACTTGA
AAAAGTGGCACCGAGTCGGTGCTTTTTTTCTAGACCCAGCTTTCTTGTACAAAGT
TGGCATTACGCTTGTGGAATTCCTCGAGGGAGTGATCAAAAGTCCCACATCGATC
AGGTGATATATAGCAGCTTAGTTTATATAATGATAGAGTCGACATAGCGATTAAA
ATAGCTCAAATCGGAGGGTTTTAGAGCTAGAAATAGCAAGTTAAAATAAGGCTA
GTCCGTTATCAACTTGAAAAAGTGGCACCGAGTCGGTGCTTTTTTTCTAGACCCA
GCTTTCTTGTACAAAGTTGGCATTACGCTGAGCGAATTCCATATGGGAGTGATCA
AAAGTCCCACATCGATCAGGTGATATATAGCAGCTTAGTTTATATAATGATAGAG
TCGACATAGCGATTGAATGTGGAGCTAAATGTAAGTTTTAGAGCTAGAAATAGC
AAGTTAAAATAAGGCTAGTCCGTTATCAACTTGAAAAAGTGGCACCGAGTCGGT
GCTTTTTTTCTAGACCCAGCTTTCTTGTACAAAGTTGGCATTACGCTTGCCGAATT
CGGATCCGGAGTGATCAAAAGTCCCACATCGATCAGGTGATATATAGCAGCTTA
GTTTATATAATGATAGAGTCGACATAGCGATTGGTGTAGGTACTACCTAAAAGGT
TTTAGAGCTAGAAATAGCAAGTTAAAATAAGGCTAGTCCGTTATCAACTTGAAA
AAGTGGCACCGAGTCGGTGCTTTTTTTCTAGACCCAGCTTTCTTGTACAAAGTTG
GCATTACGCTGCAAGAATTCAAGCTTGGAGTGATCAAAAGTCCCACATCGATCA
GGTGATATATAGCAGCTTAGTTTATATAATGATAGAGTCGACATAGCGATTGTAG
AGATTGTTTGTAATAAGTTTTAGAGCTAGAAATAGCAAGTTAAAATAAGGCTAGT
CCGTTATCAACTTGAAAAAGTGGCACCGAGTCGGTGCTTTTTTTCTAGACCCAGC
TTTCTTGTACAAAGTTGGCATTACGCTACTAGAATTCGAGCTCGGAGTGATCAAA
AGTCCCACATCGATCAGGTGATATATAGCAGCTTAGTTTATATAATGATAGAGTC
GACATAGCGATTGGTGGTAGTAATTGTGAGTAGTTTTAGAGCTAGAAATAGCAA
GTTAAAATAAGGCTAGTCCGTTATCAACTTGAAAAAGTGGCACCGAGTCGGTGCT
TTTTTTCTAGACCCAGCTTTCTTGTACAAAGTTGGCATTACGCTTTACGAGGATGC
ACATGTGACCGAGGGACACGAAGTGATCCGTTTAAACTATCAGTGTTTGACAGG
ATATATTGGCGGGTAAACCTAAGAGAAAAGAGCGTTTATTAGAATAATCGGATA
TTTAAAAGGGCGTGAAAAGGTTTATCCGTTCGTCCATTTGTATGTGCCAGCCGTG
CGGCTGCATGAAATCCTGGCCGGTTTGTCTGATGCCAAGCTGGCGGCCTGGCCGG
CCAGCTTGGCCGCTGAAGAAACCGAGCGCCGCCGTCTAAAAAGGTGATGTGTAT
TTGAGTAAAACAGCTTGCGTCATGCGGTCGCTGCGTATATGATGCGATGAGTAAA
TAAACAAATACGCAAGGGGAACGCATGAAGGTTATCGCTGTACTTAACCAGAAA
GGCGGGTCAGGCAAGACGACCATCGCAACCCATCTAGCCCGCGCCCTGCAACTC
GCCGGGGCCGATGTTCTGTTAGTCGATTCCGATCCCCAGGGCAGTGCCCGCGATT
GGGCGGCCGTGCGGGAAGATCAACCGCTAACCGTTGTCGGCATCGACCGCCCGA
CGATTGACCGCGACGTGAAGGCCATCGGCCGGCGCGACTTCGTAGTGATCGACG
GAGCGCCCCAGGCGGCGGACTTGGCTGTGTCCGCGATCAAGGCAGCCGACTTCG
TGCTGATTCCGGTGCAGCCAAGCCCTTACGACATATGGGCCACCGCCGACCTGGT
GGAGCTGGTTAAGCAGCGCATTGAGGTCACGGATGGAAGGCTACAAGCGGCCTT
TGTCGTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCGTGAGC
TACCCAGGCACTGCCGCCGCCGGCACAACCGTTCTTGAATCAGAACCCGAGGGC
GACGCTGCCCGCGAGGTCCAGGCGCTGGCCGCTGAAATTAAATCAAAACTCATTT
GAGTTAATGAGGTAAAGAGAAAATGAGCAAAAGCACAAACACGCTAAGTGCCG
GCCGTCCGAGCGCACGCAGCAGCAAGGCTGCAACGTTGGCCAGCCTGGCAGACA
CGCCAGCCATGAAGCGGGTCAACTTTCAGTTGCCGGCGGAGGATCACACCAAGC
TGAAGATGTACGCGGTACGCCAAGGCAAGACCATTACCGAGCTGCTATCTGAAT
ACATCGCGCAGCTACCAGAGTAAATGAGCAAATGAATAAATGAGTAGATGAATT
TTAGCGGCTAAAGGAGGCGGCATGGAAAATCAAGAACAACCAGGCACCGACGC
CGTGGAATGCCCCATGTGTGGAGGAACGGGCGGTTGGCCAGGCGTAAGCGGCTG
GGTTGTCTGCCGGCCCTGCAATGGCACTGGAACCCCCAAGCCCGAGGAATCGGC
GTGACGGTCGCAAACCATCCGGCCCGGTACAAATCGGCGCGGCGCTGGGTGATG
ACCTGGTGGAGAAGTTGAAGGCCGCGCAGGCCGCCCAGCGGCAACGCATCGAGG
CAGAAGCACGCCCCGGTGAATCGTGGCAAGCGGCCGCTGATCGAATCCGCAAAG
AATCCCGGCAACCGCCGGCAGCCGGTGCGCCGTCGATTAGGAAGCCGCCCAAGG
GCGACGAGCAACCAGATTTTTTCGTTCCGATGCTCTATGACGTGGGCACCCGCGA
TAGTCGCAGCATCATGGACGTGGCCGTTTTCCGTCTGTCGAAGCGTGACCGACGA
GCTGGCGAGGTGATCCGCTACGAGCTTCCAGACGGGCACGTAGAGGTTTCCGCA
GGGCCGGCCGGCATGGCCAGTGTGTGGGATTACGACCTGGTACTGATGGCGGTTT
CCCATCTAACCGAATCCATGAACCGATACCGGGAAGGGAAGGGAGACAAGCCCG
GCCGCGTGTTCCGTCCACACGTTGCGGACGTACTCAAGTTCTGCCGGCGAGCCGA
TGGCGGAAAGCAGAAAGACGACCTGGTAGAAACCTGCATTCGGTTAAACACCAC
GCACGTTGCCATGCAGCGTACGAAGAAGGCCAAGAACGGCCGCCTGGTGACGGT
ATCCGAGGGTGAAGCCTTGATTAGCCGCTACAAGATCGTAAAGAGCGAAACCGG
GCGGCCGGAGTACATCGAGATCGAGCTAGCTGATTGGATGTACCGCGAGATCAC
AGAAGGCAAGAACCCGGACGTGCTGACGGTTCACCCCGATTACTTTTTGATCGAT
CCCGGCATCGGCCGTTTTCTCTACCGCCTGGCACGCCGCGCCGCAGGCAAGGCAG
AAGCCAGATGGTTGTTCAAGACGATCTACGAACGCAGTGGCAGCGCCGGAGAGT
TCAAGAAGTTCTGTTTCACCGTGCGCAAGCTGATCGGGTCAAATGACCTGCCGGA
GTACGATTTGAAGGAGGAGGCGGGGCAGGCTGGCCCGATCCTAGTCATGCGCTA
CCGCAACCTGATCGAGGGCGAAGCATCCGCCGGTTCCTAATGTACGGAGCAGAT
GCTAGGGCAAATTGCCCTAGCAGGGGAAAAAGGTCGAAAAAGCTTCTTTCCTGT
GGATAGCACGTACATTGGGAACCCAAAGCCGTACATTGGGAACCGGAACCCGTA
CATTGGGAACCCAAAGCCGTACATTGGGAACCGGTCACACATGTAAGTGACTGA
TATAAAAGAGAAAAAAGGCGATTTTTCCGCCTAAAACTCTTTAAAACTTATTAAA
ACTCTTAAAACCCGCCTGGCCTGTGCATAACTGTCTGGCCAGCGCACAGCCGAAC
AGCTGCAAAAAGCGCCTACCCTTCGGTCGCTGCGCTCCCTACGCCCCGCCGCTTC
GCGTCGGCCTATCGCGGCCGCTGGCCGCTCAAAAATGGCTGGCCTACGGCCAGG
CAATCTACCAGGGCGCGGACAAGCCGCGCCGTCGCCACTCGACCGCCGGCGCCC
ACATCAAGGCTCCGAGTGCGCGGAACCCCTATTTGTTTATTTTTCTAAATACATTC
AAATATGTATCCGCTCATGAGACAATAACCCTGATAAATGCTTCAATAATATTGA
AAAAGGAAGAGTATGGCTAAAATGAGAATATCACCGGAATTGAAAAAACTGATC
GAAAAATACCGCTGCGTAAAAGATACGGAAGGAATGTCTCCTGCTAAGGTATAT
AAGCTGGTGGGAGAAAATGAAAACCTATATTTAAAAATGACGGACAGCCGGTAT
AAAGGGACCACCTATGATGTGGAACGGGAAAAGGACATGATGCTATGGCTGGAA
GGAAAGCTGCCTGTTCCAAAGGTCCTGCACTTTGAACGGCATGATGGCTGGAGC
AATCTGCTCATGAGTGAGGCCGATGGCGTCCTTTGCTCGGAAGAGTATGAAGATG
AACAAAGCCCTGAAAAGATTATCGAGCTGTATGCGGAGTGCATCAGGCTCTTTCA
CTCCATCGACATATCGGATTGTCCCTATACGAATAGCTTAGACAGCCGCTTAGCC
GAATTGGATTACTTACTGAATAACGATCTGGCCGATGTGGATTGCGAAAACTGGG
AAGAGGACACTCCATTTAAAGATCCGCGCGAGCTGTATGATTTTTTAAAGACGGA
AAAGCCCGAAGAGGAACTTGTCTTTTCCCACGGCGACCTGGGAGACAGCAACAT
CTTTGTGAAAGATGGCAAAGTAAGTGGCTTTATTGATCTTGGGAGAAGCGGCAG
GGCGGACAAGTGGTATGACATTGCCTTCTGCGTCCGGTCGCTCAGGGAGGATATC
GGGGAAGAACAGTATGTCGAGCTATTTTTTGACTTACTGGGGATCAAGCCTGATT
GGGAGAAAATAAAATATTATATTTTACTGGATGAATTGTTTTAGCTGTCAGACCA
AGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGC
TTCAGCAGAGCGCAGATACCAAATACTGTTCTTCTAGTGTAGCCGTAGTTAGGCC
ACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCGGCCT
TTTTACGGTTCCTGCTCGGATCTGTTGGACCGGACAGTAGTCATGGTTGATGGGC
TGCCTGTATCGAGTGGTGATTTTGTGCCGAGCTGCCGGTCGGGGAGCTGTTGGCT
GGCTGGTGGCAGGATATATTGTGGTGTAAACAAATTGACGCTTAGACAACTTAAT
AACACATTGCGGACGTTTTTAATGTACTGGGGTTGAACACTCT

### Example 3: Generation of Mutated Promoters Using CRISPR/Cas9 in Maize

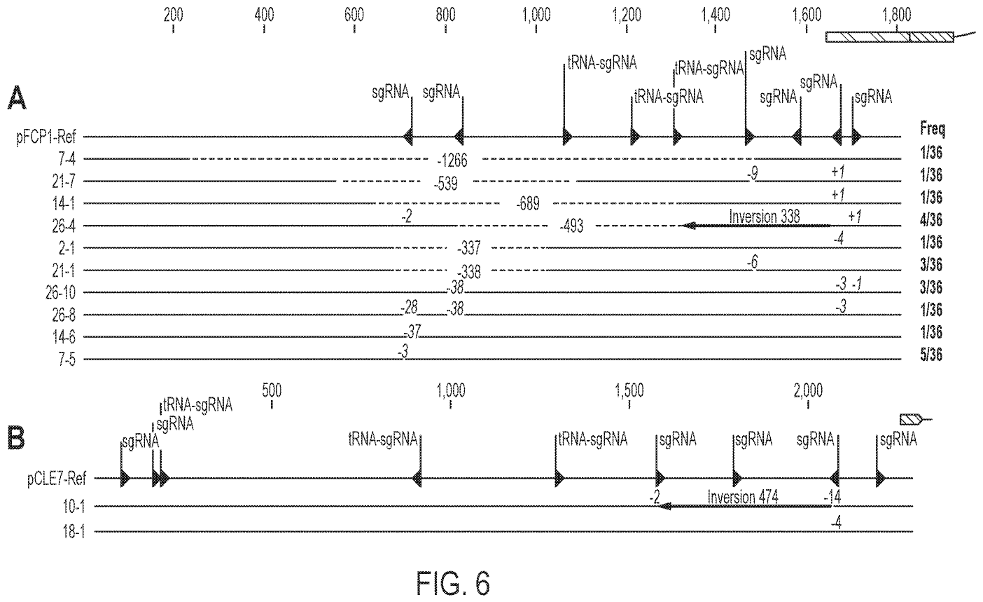

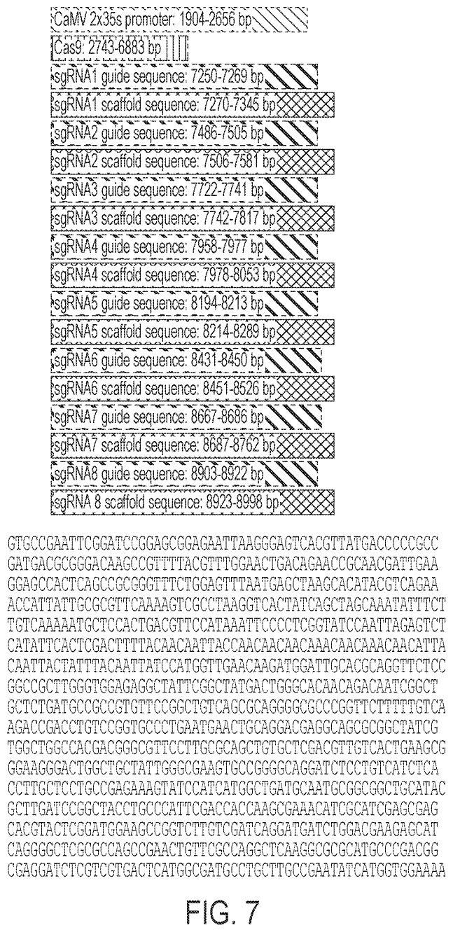

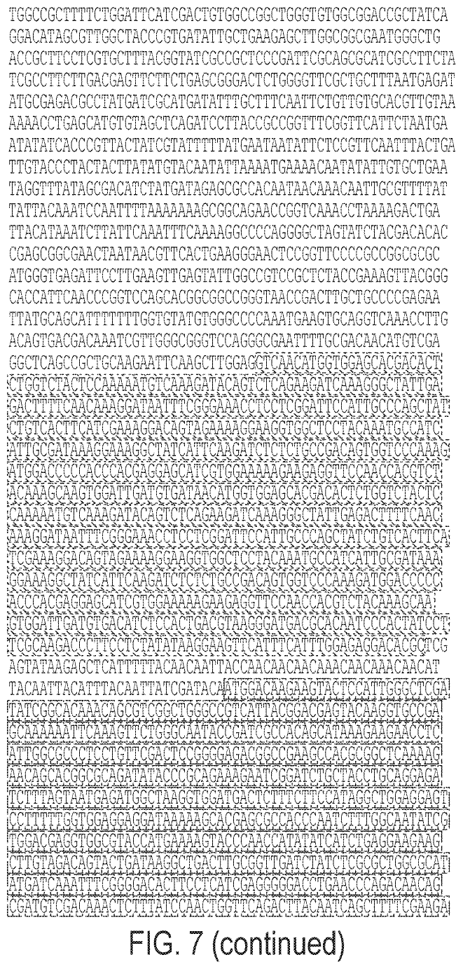

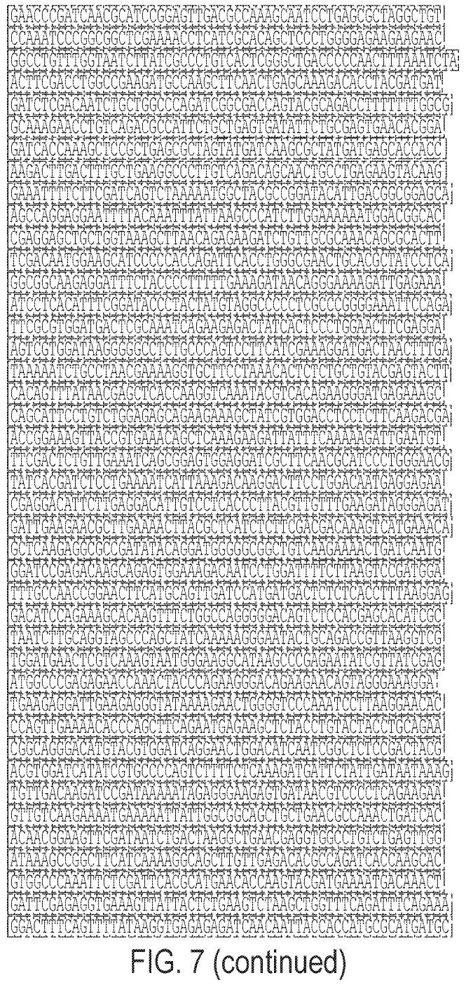

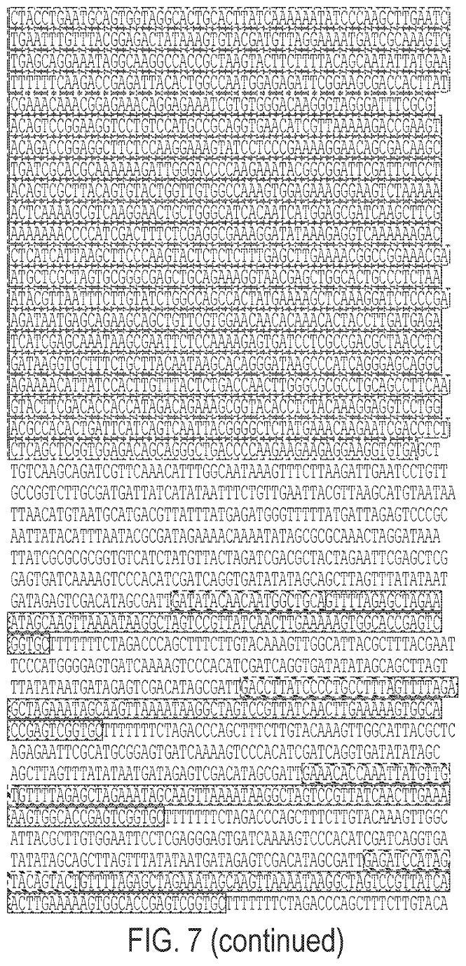

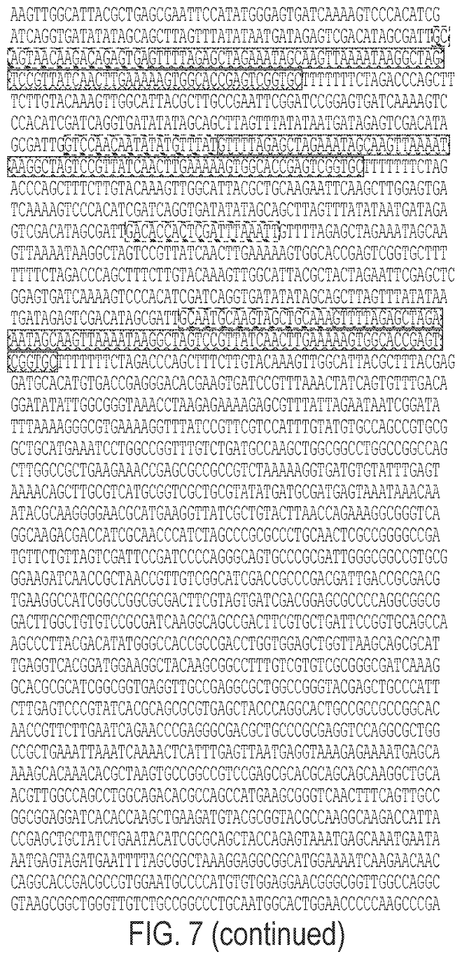

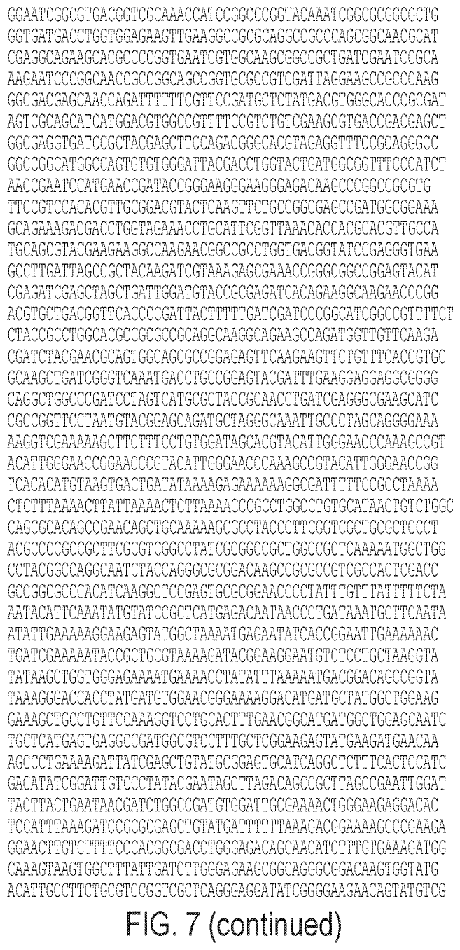

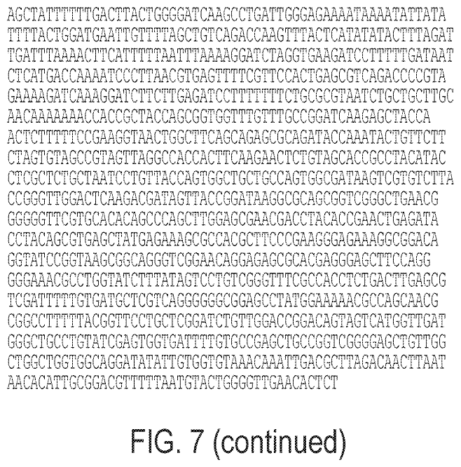

In maize, 2 promoters were targeted, the promoter of ZmCLE7, a putative CLV3 ortholog, and the promoter of ZmFCP1, a gene encoding a related CLE peptide (Je et al, 2016). sgRNA arrays for maize were constructed by DNA synthesis, and cloned by Gateway recombination into a maize transformation vector containing a rice optimized Cas9 driven by the maize ubiquitin promoter (see, e.g., Char S N, Neelakandan A K, Nahampun H, Frame B, Main M, Spalding M H, Becraft P W, Meyers B C, Walbot V, Wang K, Yang B (2017) An Agrobacterium-delivered CRISPR/Cas9 system for high-frequency targeted mutagenesis in maize. Plant Biotechnology Journal 15: 257-268). The gRNAs were expressed using different rice or maize U6 promoters, or using a polycistronic tRNA system (see, e.g., Xie, K, Minkenberg, B, Yang, Y. (2015). Boosting CRISPR/Cas9 multiplex editing capability with the endogenous tRNA-processing system. Proc Natl Acad Sci USA. 2015; 112: 3570-5). The constructs were transformed into maize and transgenic seedlings were obtained for molecular analysis. DNA sequencing revealed various promoter mutations, including small indels, larger deletions and inversions (FIG. 6), illustrating that the promoter CRISPR method also works well in maize. The lines including the various promoter mutations were propagated. The lines are then crossed to null mutants of Zmfcp1 or cle7 for phenotypic analysis. The annotated sequences of the sgRNA arrays for both the ZmFCP1 promoter and the ZmCLE7 promoter are shown below.

Annotated ZmCLE7 Promoter CRISPR sgRNA Array

ZmpU6C1 promoter sequence: 1-178 bp
sgRNA1 guide sequence: 179-198 bp
sgRNA1 scaffold sequence: 199-274 bp
Terminator sequence: 275-282 bp
ZmpU6C3 promoter sequence: 288-481 bp
sgRNA2 guide sequence: 482-501 bp
sgRNA2 scaffold sequence: 502-577 bp
Terminator sequence: 578-584 bp
Rice U6.1 promoter sequence: 585-917 bp
tRNA sequence: 918-995 bp
sgRNA3 guide sequence: 996-1014 bp
sgRNA3 scaffold sequence: 1015-1090 bp
tRNA sequence: 1091-1167 bp
sgRNA4 guide sequence: 1168-1187 bp
sgRNA4 scaffold sequence: 1188-1263 bp
tRNA sequence: 1264-1340 bp
sgRNA5 guide sequence: 1341-1360 bp
sgRNA5 scaffold sequence: 1361-1436 bp
Terminator sequence: 1437-1445 bp
Rice pU6.2 promoter sequence: 1446-1690 bp
sgRNA6 guide sequence: 1691-1710 bp
sgRNA6 scaffold sequence: 1711-1786 bp
Terminator sequence: 1787-1793 bp
ZmpU6C1 promoter sequence: 1800-1977 bp
sgRNA7 guide sequence: 1978-1998 bp
sgRNA7 scaffold sequence: 1999-2074 bp
Terminator sequence: 2075-2082 bp
ZmpU6C3 promoter sequence: 2088-2281 bp
sgRNA8 guide sequence: 2282-2301 bp
sgRNA8 scaffold sequence: 2302-2377 bp
Terminator sequence: 2378-2384 bp
Rice pU6.2 promoter sequence: 2391-2635 bp
sgRNA9 guide sequence: 2636-2655 bp
sgRNA9 scaffold sequence: 2656-2731 bp
Terminator sequence: 2732-2738 bp
(SEQ ID NO: 4)
CACGTGAGCTTGCGATGTCCACTAGGGAGCTCCATCCACTGATCCACCCCCACGC
GGCGTGGCGTCGTCATTAACGGCTTGTGGGGAAGGGAACGAGCAACTAACCGAT
AATTAGTACCAGACCGGCCAGTGAACGATGCCAAAACCGGCTTATAAGCTCAGC
TGCGACAACCGTTTTCACGACACGGAACAATTAAGGTTTTAGAGCTAGAAATAG
CAAGTTAAAATAAGGCTAGTCCGTTATCAACTTGAAAAAGTGGCACCGAGTCGG
TGCTTTTTTTTACGTACAAAAACATCCTCACAGGAAAGACACGAAGAAACATGGI
CAATGGCCCATTATATAAAGCACCGCCACAAAGCCCAAATACCAGTTCGTCGGT
GGAGCAAGTAACGCGCTAGGCAACAGGCAAACAGTTTGTCCCACCTCGTCCAGT
CACAAAGGCAAAGCGTGACTTATAAGCCAGAGCGGAAGAACCATACCCCGCCCG
TTTGGACATATATGTTTTAGAGCTAGAAATAGCAAGTTAAAATAAGGCTAGTCCG
TTATCAACTTGAAAAAGTGGCACCGAGTCGGTGCTTTTTTTGTTAACTAAGAACG
AACTAAGCCGGACAAAAAAAGGAGCACATATACAAACCGGTTTTATTCATGAAT
GGTCACGATGGATGATGGGGCTCAGACTTGAGCTACGAGGCCGCAGGCGAGAGA
AGCCTAGTGTGCTCTCTGCTTGTTTGGGCCGTAACGGAGGATACGGCCGACGAGC
GTGTACTACCGCGCGGGATGCCGCTGGGCGCTGCGGGGGCCGTTGGATGGGGAT
CGGTGGGTCGCGGGAGCGTTGAGGGGAGACAGGTTTAGTACCACCTCGCCTACC
GAACAATGAAGAACCCACCTTATAACCCCGCGCGCTGCCGCTTGTGTTGAACAA
AGCACCAGTGGTCTAGTGGTAGAATAGTACCCTGCCACGGTACAGACCCGGGTT
CGATTCCCGGCTGGTGCAGGTAGATCGCGTGCGTACAGTTTTAGAGCTAGAAATA
GCAAGTTAAAATAAGGCTAGTCCGTTATCAACTTGAAAAAGTGGCACCGAGTCG
GTGCAACAAAGCACCAGTGGTCTAGTGGTAGAATAGTACCCTGCCACGGTACAG
ACCCGGGTTCGATTCCCGGCTGGTGCAGACACGGACACAGTGGCACCGTTTTAGA
GCTAGAAATAGCAAGTTAAAATAAGGCTAGTCCGTTATCAACTTGAAAAAGTGG
CACCGAGTCGGTGCAACAAAGCACCAGTGGTCTAGTGGTAGAATAGTACCCTGC
CACGGTACAGACCCGGGTTCGATTCCCGGCTGGTGCAGATACCCGTATAGACAA
GTTGTTTTAGAGCTAGAAATAGCAAGTTAAAATAAGGCTAGTCCGTTATCAACTT
GAAAAAGTGGCACCGAGTCGGTGCTTTTTTTTTGGATCATGAACCAACGGCCTGG
CTGTATTTGGTGGTTGTGTAGGGAGATGGGGAGAAGAAAAGCCCGATTCTCTTCG
CTGTGATGGGCTGGATGCATGCGGGGGAGCGGGAGGCCCAAGTACGTGCACGGT
GAGCGGCCCACAGGGCGAGTGTGAGCGCGAGAGGCGGGAGGAACAGTTTAGTA
CCACATTGCCCAGCTAACTCGAACGCGACCAACTTATAAACCCGCGCGCTGTCGC
TTGTGTGCTTGTACTTTACTCCGTAGGTTTTAGAGCTAGAAATAGCAAGTTAAAA
TAAGGCTAGTCCGTTATCAACTTGAAAAAGTGGCACCGAGTCGGTGCTTTTTTTG
TTAACCACGTGAGCTTGCGATGTCCACTAGGGAGCTCCATCCACTGATCCACCCC
CACGCGGCGTGGCGTCGTCATTAACGGCTTGTGGGGAAGGGAACGAGCAACTAA
CCGATAATTAGTACCAGACCGGCCAGTGAACGATGCCAAAACCGGCTTATAAGC
TCAGCTGCGACAACCGTTTTGCTTTCCAAACTGATGCGTACGTTTTAGAGCTAGA
AATAGCAAGTTAAAATAAGGCTAGTCCGTTATCAACTTGAAAAAGTGGCACCGA
GTCGGTGCTTTTTTTTACGTACAAAAACATCCTCACAGGAAAGACACGAAGAAAC
ATGGTCAATGGCCCATTATATAAAGCACCGCCACAAAGCCCAAATACCAGTTCGT
CGGTGGAGCAAGTAACGCGCTAGGCAACAGGCAAACAGTTTGTCCCACCTCGTC
CAGTCACAAAGGCAAAGCGTGACTTATAAGCCAGAGCGGAAGAACCATACCGGG
GCCGCGGCGGTACTTATGTTTTAGAGCTAGAAATAGCAAGTTAAAATAAGGCTA
GTCCGTTATCAACTTGAAAAAGTGGCACCGAGTCGGTGCTTTTTTTGTTAACGGA
TCATGAACCAACGGCCTGGCTGTATTTGGTGGTTGTGTAGGGAGATGGGGAGAA
GAAAAGCCCGATTCTCTTCGCTGTGATGGGCTGGATGCATGCGGGGGAGCGGGA
GGCCCAAGTACGTGCACGGTGAGCGGCCCACAGGGCGAGTGTGAGCGCGAGAG
GCGGGAGGAACAGTTTAGTACCACATTGCCCAGCTAACTCGAACGCGACCAACT
TATAAACCCGCGCGCTGTCGCTTGTGTGTTATACACACCGCGGTTTTGTTTTAGAG
CTAGAAATAGCAAGTTAAAATAAGGCTAGTCCGTTATCAACTTGAAAAAGTGGC
ACCGAGTCGGTGCTTTTTTTGTTAAC

Annotated ZmFCP1 Promoter CRISPR sgRNA Array

ZmpU6C1 promoter sequence: 1-178 bp
sgRNA1 guide sequence: 179-198 bp
sgRNA1 scaffold sequence: 199-274 bp
Terminator sequence: 275-282 bp
ZmpU6C3 promoter sequence: 288-481 bp
sgRNA2 guide sequence: 482-501 bp
sgRNA2 scaffold sequence: 502-577 bp
Terminator sequence: 578-584 bp
Rice U6.1 promoter sequence: 585-918 bp
tRNA sequence: 919-995 bp
sgRNA3 guide sequence: 996-1015 bp
sgRNA3 scaffold sequence: 1016-1091 bp
tRNA sequence: 1092-1168 bp
sgRNA4 guide sequence: 1169-1188 bp
sgRNA4 scaffold sequence: 1189-1264 bp
tRNA sequence: 1265-1341 bp
sgRNA5 guide sequence: 1342-1361 bp
sgRNA5 scaffold sequence: 1362-1437 bp
Terminator sequence: 1438-1446 bp
Rice pU6.2 promoter sequence: 1447-1691 bp
sgRNA6 guide sequence: 1692-1711 bp
sgRNA6 scaffold sequence: 1712-1787 bp
Terminator sequence: 1788-1794 bp
ZmpU6C1 promoter sequence: 1801-1978 bp
sgRNA7 guide sequence: 1979-1998 bp
sgRNA7 scaffold sequence: 1999-2074 bp
Terminator sequence: 2075-2082 bp
ZmpU6C3 promoter sequence: 2088-2281 bp
sgRNA8 guide sequence: 2282-2301 bp
sgRNA8 scaffold sequence: 2302-2377 bp
Terminator sequence: 2378-2384 bp
Rice pU6.2 promoter sequence: 2391-2635 bp
sgRNA9 guide sequence: 2636-2655 bp
sgRNA9 scaffold sequence: 2656-2731 bp
Terminator sequence: 2732-2738 bp
(SEQ ID NO: 5)
CACGTGAGCTTGCGATGTCCACTAGGGAGCTCCATCCACTGATCCACCCCCACGC
GGCGTGGCGTCGTCATTAACGGCTTGTGGGGAAGGGAACGAGCAACTAACCGAT
AATTAGTACCAGACCGGCCAGTGAACGATGCCAAAACCGGCTTATAAGCTCAGC
TGCGACAACCGTTTTGGTCAAGAGCAACCAAACAAGTTTTAGAGCTAGAAATAG
CAAGTTAAAATAAGGCTAGTCCGTTATCAACTTGAAAAAGTGGCACCGAGTCGG
TGCTTTTTTTTACGTACAAAAACATCCTCACAGGAAAGACACGAAGAAACATGGT
CAATGGCCCATTATATAAAGCACCGCCACAAAGCCCAAATACCAGTTCGTCGGT
GGAGCAAGTAACGCGCTAGGCAACAGGCAAACAGTTTGTCCCACCTCGTCCAGT
CACAAAGGCAAAGCGTGACTTATAAGCCAGAGCGGAAGAACCATACCGCACCAG
TAGAGATTGGCTCGTTTTAGAGCTAGAAATAGCAAGTTAAAATAAGGCTAGTCC
GTTATCAACTTGAAAAAGTGGCACCGAGTCGGTGCTTTTTTTGTTAACTAAGAAC
GAACTAAGCCGGACAAAAAAAGGAGCACATATACAAACCGGTTTTATTCATGAA
TGGTCACGATGGATGATGGGGCTCAGACTTGAGCTACGAGGCCGCAGGCGAGAG
AAGCCTAGTGTGCTCTCTGCTTGTTTGGGCCGTAACGGAGGATACGGCCGACGAG
CGTGTACTACCGCGCGGGATGCCGCTGGGCGCTGCGGGGGCCGTTGGATGGGGA
TCGGTGGGTCGCGGGAGCGTTGAGGGGAGACAGGTTTAGTACCACCTCGCCTAC
CGAACAATGAAGAACCCACCTTATAACCCCGCGCGCTGCCGCTTGTGTTGAACAA
AGCACCAGTGGTCTAGTGGTAGAATAGTACCCTGCCACGGTACAGACCCGGGTT
CGATTCCCGGCTGGTGCAGGCTCGACCATGTTCAGACTGTTTTAGAGCTAGAAAT
AGCAAGTTAAAATAAGGCTAGTCCGTTATCAACTTGAAAAAGTGGCACCGAGTC
GGTGCAACAAAGCACCAGTGGTCTAGTGGTAGAATAGTACCCTGCCACGGTACA
GACCCGGGTTCGATTCCCGGCTGGTGCAGCACTTCCACTTTGGTTTTGGTTTTAGA
GCTAGAAATAGCAAGTTAAAATAAGGCTAGTCCGTTATCAACTTGAAAAAGTGG
CACCGAGTCGGTGCAACAAAGCACCAGTGGTCTAGTGGTAGAATAGTACCCTGC
CACGGTACAGACCCGGGTTCGATTCCCGGCTGGTGCAGCGAAAAGGAATCCATG
CTGGTTTTAGAGCTAGAAATAGCAAGTTAAAATAAGGCTAGTCCGTTATCAACTT
GAAAAAGTGGCACCGAGTCGGTGCTTTTTTTTTGGATCATGAACCAACGGCCTGG
CTGTATTTGGTGGTTGTGTAGGGAGATGGGGAGAAGAAAAGCCCGATTCTCTTCG
CTGTGATGGGCTGGATGCATGCGGGGGAGCGGGAGGCCCAAGTACGTGCACGGT
GAGCGGCCCACAGGGCGAGTGTGAGCGCGAGAGGCGGGAGGAACAGTTTAGTA
CCACATTGCCCAGCTAACTCGAACGCGACCAACTTATAAACCCGCGCGCTGTCGC
TTGTGTGATCGCGGGTCCCACGCATAGTTTTAGAGCTAGAAATAGCAAGTTAAAA
TAAGGCTAGTCCGTTATCAACTTGAAAAAGTGGCACCGAGTCGGTGCTTTTTTTG
TTAACCACGTGAGCTTGCGATGTCCACTAGGGAGCTCCATCCACTGATCCACCCC
CACGCGGCGTGGCGTCGTCATTAACGGCTTGTGGGGAAGGGAACGAGCAACTAA
CCGATAATTAGTACCAGACCGGCCAGTGAACGATGCCAAAACCGGCTTATAAGC
TCAGCTGCGACAACCGTTTTGTGGTACGGTCACGTGCCGCGTTTTAGAGCTAGAA
ATAGCAAGTTAAAATAAGGCTAGTCCGTTATCAACTTGAAAAAGTGGCACCGAG
TCGGTGCTTTTTTTTACGTACAAAAACATCCTCACAGGAAAGACACGAAGAAACA
TGGTCAATGGCCCATTATATAAAGCACCGCCACAAAGCCCAAATACCAGTTCGTC
GGTGGAGCAAGTAACGCGCTAGGCAACAGGCAAACAGTTTGTCCCACCTCGTCC
AGTCACAAAGGCAAAGCGTGACTTATAAGCCAGAGCGGAAGAACCATACCGAG
AGTTGGTTTCGCCCGTCGTTTTAGAGCTAGAAATAGCAAGTTAAAATAAGGCTAG
TCCGTTATCAACTTGAAAAAGTGGCACCGAGTCGGTGCTTTTTTTGTTAACGGAT
CATGAACCAACGGCCTGGCTGTATTTGGTGGTTGTGTAGGGAGATGGGGAGAAG
AAAAGCCCGATTCTCTTCGCTGTGATGGGCTGGATGCATGCGGGGGAGCGGGAG
GCCCAAGTACGTGCACGGTGAGCGGCCCACAGGGCGAGTGTGAGCGCGAGAGGC
GGGAGGAACAGTTTAGTACCACATTGCCCAGCTAACTCGAACGCGACCAACTTA
TAAACCCGCGCGCTGTCGCTTGTGTGGTTTTGGAGCAGGCAAGCCGTTTTAGAGC
TAGAAATAGCAAGTTAAAATAAGGCTAGTCCGTTATCAACTTGAAAAAGTGGCA
CCGAGTCGGTGCTTTTTTTGTTAAC

### REFERENCES

Brooks, C., Nekrasov, V., Lippman, Z. B., and Van Eck, J. (2014). Efficient Gene Editing in Tomato in the First Generation Using the Clustered Regularly Interspaced Short Palindromic Repeats/CRISPR-Cas9 System. Plant Physiol 166: 1292-1297.
Gupta, S. and Van Eck, J. (2016). Modification of plant regeneration medium decreases the time for recovery of Solanum lycopersicum cultivar M82 stable transgenic lines. Plant Cell. Tissue Organ Cult. 127: 417-423.
Lei, Y., Lu, L., Liu, H.-Y., Li, S., Xing, F., and Chen, L.-L. (2014). CRISPR-P: A Web Tool for Synthetic Single-Guide RNA Design of CRISPR-System in Plants. Mol. Plant: 1-3.
Werner, S., Engler, C., Weber, E., Gruetzner, R., and Marillonnet, S. (2012). Fast track assembly of multigene constructs using Golden Gate cloning and the MoClo system. Bioengineered 3: 38-43.
King, M.-C. and A. C. Wilson (1975). Evolution at two levels in humans and chimpanzees. Science. 188(4184): p. 107-116.
Olsen, K. M. and J. F. Wendel (2013). Crop plants as models for understanding plant adaptation and diversification. Frontiers in Plant Science. 4:290.
Doebley, J. (2006). Plant science. Unfallen grains: how ancient farmers turned weeds into crops. Science. 312(5778): p. 1318-1319.
Meyer, R. S. and M. D. Purugganan (2013). Evolution of crop species: genetics of domestication and diversification. Nature reviews Genetics. 14(12): p. 840-52.
Xu, C., K. L. Liberatore, C. A. MacAlister, Z. Huang, Y. H. Chu, K. Jiang, C. Brooks, M. Ogawa-Ohnishi, G. Xiong, M. Pauly, J. Van Eck, Y. Matsubayashi, E. van der Knaap, and Z. Lippman (2015). A cascade of arabinosyltransferases controls shoot meristem size in tomato. Nature Genetics. DOI: 10.1038/ng.3309.
Carroll, S. (2000). Endless forms: the evolution of gene regulation and morphological diversity. Cell. 101(6): p. 577.
Carroll, S. B. (2008). Evo-devo and an expanding evolutionary synthesis: A genetic theory of morphological evolution. Cell. 134(1): p. 25-36.
Stern, D. L. (2000). Perspective: evolutionary developmental biology and the problem of variation. Evolution. 54(4): p. 1079-1091.
Frankel, N., G. K. Davis, D. Vargas, S. Wang, F. Payre, and D. L. Stern (2010). Phenotypic robustness conferred by apparently redundant transcriptional enhancers. Nature. 466(7305): p. 490-493.
Bommert, P., N. S. Nagasawa, and D. Jackson (2013). Quantitative variation in maize kernel row number is controlled by the FASCIATED EAR2 locus. Nature Genetics. 45: p. 334-337.
Sandelin, A., W. Alkema, P. Engström, W. W. Wasserman, and B. Lenhard (2004). JASPAR: an open-access database for eukaryotic transcription factor binding profiles. Nucleic acids research. 32(suppl 1): p. D91-D94.
Turco, G., J. C. Schnable, B. Pedersen, and M. Freeling (2013). Automated conserved non-coding sequence (CNS) discovery reveals differences in gene content and promoter evolution among grasses. Frontiers in plant science. 4: 170.
O'Connor, T. R., C. Dyreson, and J. J. Wyrick (2005). Athena: a resource for rapid visualization and systematic analysis of Arabidopsis promoter sequences. Bioinformatics. 21(24): p. 4411-3.
Baxter, L., A. Jironkin, R. Hickman, J. Moore, C. Barrington, P. Krusche, N. P. Dyer, V. Buchanan-Wollaston, A. Tiskin, J. Beynon, K. Denby, and S. Ott (2012). Conserved noncoding sequences highlight shared components of regulatory networks in dicotyledonous plants. Plant Cell. 24(10): p. 3949-65.
Haudry, A., A. E. Platts, E. Vello, D. R. Hoen, M. Leclercq, R. J. Williamson, E. Forczek, Z. Joly-Lopez, J. G. Steffen, K. M. Hazzouri, K. Dewar, J. R. Stinchcombe, D. J. Schoen, X. Wang, J. Schmutz, C. D. Town, P. P. Edger, J. C. Pires, K. S. Schumaker, D. E. Jarvis, T. Mandakova, M. A. Lysak, E. van den Bergh, M. E. Schranz, P. M. Harrison, A. M. Moses, T. E. Bureau, S. I. Wright, and M. Blanchette (2013). An atlas of over 90,000 conserved noncoding sequences provides insight into crucifer regulatory regions. Nat Genet. 45(8): p. 891-8.
Matys, V., E. Fricke, R. Geffers, E. Gößling, M. Haubrock, R. Hehl, K. Hornischer, D. Karas, A. E. Kel, and O. V. Kel-Margoulis (2003). TRANSFAC®: transcriptional regulation, from patterns to profiles. Nucleic acids research. 31(1): p. 374-378.
Bailey, T. L. (2011). DREME: motif discovery in transcription factor ChIP-seq data. Bioinformatics. 27(12): p. 1653-1659.
Korkuc, P., J. H. Schippers, and D. Walther (2014). Characterization and identification of cisregulatory elements in Arabidopsis based on single-nucleotide polymorphism information.
Plant Physiol. 164(1): p. 181-200.
Chia, J.-M., C. Song, P. J. Bradbury, D. Costich, N. de Leon, J. Doebley, R. J. Elshire, B. Gaut, L. Geller, and J. C. Glaubitz (2012). Maize HapMap2 identifies extant variation from a genome in flux. Nature genetics. 44(7): p. 803-807.
Sim, S.-C., A. Van Deynze, K. Stoffel, D. S. Douches, D. Zarka, M. W. Ganal, R. T. Chetelat, S. F. Hutton, J. W. Scott, R. G. Gardner, D. R. Panthee, M. Mutschler, J. R. Myers, and D. M. Francis (2012). High-Density SNP Genotyping of Tomato (Solanum lycopersicum L.) Reveals Patterns of Genetic Variation Due to Breeding. PLoS ONE. 7(9): p. e45520.
Kafri, R., A. Bar-Even, and Y. Pilpel (2005). Transcription control reprogramming in genetic backup circuits. Nature genetics. 37(3): p. 295-299.
Till, B. J., S. H. Reynolds, C. Weil, N. Springer, C. Burtner, K. Young, E. Bowers, C. A. Codomo, L. C. Enns, A. R. Odden, E. A. Greene, L. Comai, and S. Henikoff (2004). Discovery of induced point mutations in maize genes by TILLING. BMC plant biology. 4: p. 12.
Munos, S., N. Ranc, E. Botton, A. Berard, S. Rolland, P. Duffe, Y. Carretero, M. C. Le Paslier, C. Delalande, M. Bouzayen, D. Brunel, and M. Causse (2011). Increase in tomato locule number is controlled by two single-nucleotide polymorphisms located near WUSCHEL Plant physiology. 156(4): p. 2244-54.
van der Knaap, E., M. Chakrabarti, Y. H. Chu, J. P. Clevenger, E. Illa-Berenguer, Z. Huang, N. Keyhaninejad, Q. Mu, L. Sun, Y. Wang, and S. Wu (2014). What lies beyond the eye: the molecular mechanisms regulating tomato fruit weight and shape. Frontiers in plant science. 5: p. 227.
Lei, Y., L. Lu, H. Y. Liu, S. Li, F. Xing, and L. L. Chen (2014). CRISPR-P: a web tool for synthetic single-guide RNA design of CRISPR-system in plants. Mol Plant. 7(9): p. 1494-6.
Wray, G. A., M. W. Hahn, E. Abouheif, J. P. Balhoff, M. Pizer, M. V. Rockman, and L. A. Romano (2003). The evolution of transcriptional regulation in eukaryotes. Molecular biology and evolution. 20(9): p. 1377-419.
Rombauts, S., K. Florquin, M. Lescot, K. Marchal, P. Rouze, and Y. van de Peer (2003). Computational approaches to identify promoters and cis-regulatory elements in plant genomes. Plant physiology. 132(3): p. 1162-76.
Paixao, T. and R. B. Azevedo (2010). Redundancy and the evolution of cis-regulatory element multiplicity. PLoS computational biology. 6(7): p. e1000848.
Taguchi-Shiobara, F., Z. Yuan, S. Hake, and D. Jackson (2001). The FASCIATED EAR2 gene encodes a leucine-rich repeat receptor-like protein that regulates shoot meristem proliferation in maize. Genes Dev. 15(20): p. 2755-66.
Nimchuk, Z. L., Y. Zhou, P. T. Tarr, B. A. Peterson, and E. M. Meyerowitz (2015). Plant stem cell maintenance by transcriptional cross-regulation of related receptor kinases. Development. 142(6): p. 1043-9.
Park, S. J., K. Jiang, M. C. Schatz, and Z. B. Lippman (2012). Rate of meristem maturation determines inflorescence architecture in tomato. Proc Natl Acad Sci USA. 109(2): p. 639-44.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A method of producing a plant or seed, the method comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest,
(c) crossing the first plant to the second plant to produce a plurality of F1 hybrid plants, each F1 hybrid plant in the plurality comprising the first allele, the second allele and the expression cassette,
(d) maintaining the plurality of F1 hybrid plants under conditions that permit the gRNA/RNA-guided endonuclease to induce mutations within the target region of the second allele,
(e) selecting an F1 hybrid plant of step (d) having a phenotype of interest, and
(f) performing a cross with the F1 hybrid plant to produce a progeny plant or seed containing at least one gRNA/RNA-guided endonuclease-induced mutation.

2. The method of claim 1, wherein the mutation is a deletion, inversion, translocation or insertion, or a combination of structural variations thereof.

3. The method of claim 1 or 2, wherein the method further comprises propagating or multiplying the progeny plant or seed.

4. The method of any one of claims 1 to 3, wherein the method further comprises producing a seed from the progeny plant or seed.

5. The method of any one of claims 1 to 4, wherein the RNA-guided endonuclease is a Cas9 or Cpf1 endonuclease.

6. A method of producing a plant or seed, the method comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest,
(c) crossing the first plant to the second plant to produce a plurality of F1 hybrid plants, each F1 hybrid plant in the plurality comprising the first allele, the second allele and the expression cassette,
(d) maintaining the plurality of F1 hybrid plants under conditions that permit the gRNA/RNA-guided endonuclease to induce mutations within the target region of the second allele,
(e) selecting an F1 hybrid plant of step (d) having a phenotype of interest, and
(f) performing a cross with the F1 hybrid plant to produce a progeny plant or seed that is homozygous for the second allele containing at least one gRNA/RNA-guided endonuclease-induced mutation.

7. The method of claim 6, wherein the method further comprises propagating or multiplying the progeny plant or seed.

8. The method of claim 6 or 7, wherein the method further comprises producing a seed from the progeny plant or seed.

9. The method of any one of claims 6 to 8, wherein the method further comprises isolating a cell from the plant or seed.

10. The method of any one of claims 6 to 9, wherein the method further comprises isolating a DNA molecule from the cell, wherein the isolated DNA molecule comprises the second allele of the gene of interest containing the at least one gRNA/Cas9-induced mutation or a fragment of the second allele containing the target region containing the at least one gRNA/Cas9-induced mutation.

11. The method of any one of claims 6 to 10, wherein the RNA-guided endonuclease is a Cas9 or Cpf1 endonuclease.

12. A method of generating a commercially relevant allele or trait that can be used in plant breeding, comprising
(a) selecting an F1 hybrid plant, which is hemizygous for an expression cassette that encodes a RNA-guided endonuclease and at least two different gRNAs, each gRNA containing a sequence that is complementary to a target sequence within a target region of a gene of interest, and having a first allele of the gene of interest that is a null allele or a hypomorphic allele and a second allele of the gene of interest carrying a gRNA/endonuclease-induced mutation within the promotor region of the gene of interest; and
(b) fixing the second allele in a plant to produce a progeny plant or seed that is homozygous for that second allele.

13. The method of claim 12, wherein the expression cassette encodes a Cas9 or Cpf1 endonuclease.

14. The method of claim 12 or 13, wherein the second allele is fixed in a progeny plant or seed by performing a self-cross of the F1 hybrid plant.

15. The method of any one of claims 12 to 14, wherein the progeny plant or seed does not carry the expression cassette.

16. The method of any one of claims 12 to 15, wherein the second allele is fixed in a progeny plant or seed by performing at least two outcrosses of the F1 hybrid plant with a plant that does not contain the expression cassette.

17. The method of any one of claims 12 to 16, wherein the F1 hybrid plant is a crop plant.

18. The method of any one of claims 12 to 17, wherein after step (b), the second allele is introduced into a different plant that does not contain the expression cassette to produce a different plant or seed containing the second allele, and optionally further propagating or multiplying the different plant or seed containing the second allele.

19. The method of claim 18, wherein the second allele is fixed in the different plant or seed, for the production of a plant or seed that is homozygous for the second allele.

20. A method for producing a crop plant or crop seed having a commercially relevant allele of a gene of interest, comprising using the method of any one of claims 12-19 to produce a commercially relevant allele of a gene of interest, introducing the allele into a crop plant, to produce a crop plant or crop seed containing the allele, and optionally further propagating or multiplying that crop plant or crop seed.

21. A method of generating a commercially relevant allele or trait that can be used in plant breeding, comprising
(a) selecting an F1 hybrid plant, which is hemizygous for an expression cassette that encodes a RNA guided endonuclease and at least two different gRNAs, each gRNA containing a sequence that is complementary to a target sequence within a target region of a gene of interest, and having a first allele of the gene of interest that is a null allele or a hypomorphic allele and a second allele of that gene carrying a gRNA/endonuclease induced mutation within the promotor region of that gene; and
(b) performing a cross of the F1 hybrid plant to produce a progeny plant or seed that is heterozygous for that second allele.

22. The method of claim 21, wherein the expression cassette encodes a Cas9 or Cpf1 endonuclease.

23. The method of claim 21 or 22, wherein the cross of the F1 hybrid plant is a self-cross.

24. The method of any one of claims 21 to 23, wherein the cross of the F1 hybrid plant is an outcross.

25. The method of any one of claims 21 to 24, wherein the progeny plant does not carry the expression cassette.

26. The method of any one of claims 21 to 25, wherein the F1 hybrid plant is a crop plant.

27. The method of any one of claims 21 to 26, wherein after producing the progeny plant or seed that is heterozygous for the second allele, the second allele is introduced into a different plant that does not contain the expression cassette for the production of a plant or seed, optionally further propagating or multiplying that plant or seed.

28. The method of claim 27, wherein the second allele is fixed in the different plant, for the production of a plant or seed that is homozygous for the second allele.

29. A method for producing a crop plant or crop seed having a commercially relevant allele of a gene of interest, comprising using the method of any one of claims 21-28 to produce a commercially relevant allele of a gene of interest, introducing the allele into a crop plant, to produce a crop plant or crop seed containing the allele, and optionally further propagating or multiplying that crop plant or crop seed.

30. A method of generating a plant library comprising a plurality of F1 hybrid plants, the method comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 2000 base pairs downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the expression cassette.

31. A method of generating a seed library comprising a plurality of F1 hybrid seeds, the method comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the expression cassette.

32. The method of claim 30 or 31, wherein the first plant is hemizygous for the expression cassette.

33. The method of any one of claims 30 to 32, wherein the first plant is homozygous for the first allele and the second plant is homozygous for the second allele.

34. The method of any one of claims 30 to 33, wherein the method further comprises maintaining the plurality of F1 hybrid plants or F1 hybrid seeds under conditions that permit the gRNA/endonuclease to induce mutations within the target region of the second allele.

35. The method of any one of claims 30 to 34, wherein the RNA-guided endonuclease is a Cas9 or Cpf1 endonuclease.

36. A plant library comprising a plurality of F1 hybrid plants, each F1 hybrid plant in the plurality comprising:
(a) a gene of interest comprising a coding sequence and having a first allele that is a hypomorphic allele or a null allele and a second allele that is different from the first allele, and
(b) an expression cassette that encodes a RNA-guided endonuclease and at least four different guide RNAs (gRNAs), each gRNA containing a sequence that is complementary to a target sequence within a target region in the second allele of the gene of interest,
wherein the target region is 0 to 5000 base pairs upstream of the 5′ end of the coding sequence of the gene of interest or wherein the target region is 0 to 5000 base pairs downstream of the 3′ end of the coding sequence of the gene of interest.

37. A seed library comprising a plurality of F1 hybrid seeds, each F1 hybrid seed in the plurality comprising:
(a) a gene of interest comprising a coding sequence and having a first allele that is a hypomorphic allele or a null allele and a second allele that is different from the first allele, and
(b) an expression cassette that encodes a RNA-guided endonuclease and at least four different guide RNAs (gRNAs), each gRNA containing a sequence that is complementary to a target sequence within a target region in the second allele of the gene of interest,
wherein the target region is 0 to 5000 base pairs upstream of the 5′ end of the coding sequence of the gene of interest or wherein the target region is 0 to 5000 base pairs downstream of the 3′ end of the coding sequence of the gene of interest.

38. The library of claim 36 or 37, wherein the target region comprises a regulatory region of the gene of interest.

39. The library of claim 38, wherein the regulatory region comprises a transcription factor binding site, an RNA polymerase binding site, a TATA box, or a combination of structural variations thereof.

40. The library of claim 38 or 39, wherein the regulatory region is a promoter.

41. The library of any one of claims 36 to 40, wherein the expression cassette encodes at least five different gRNAs.

42. The library of claim 41, wherein the expression cassette encodes at least six different gRNAs.

43. The library of claim 41, wherein the expression cassette encodes at least seven different gRNAs.

44. The library of claim 41, wherein the expression cassette encodes at least eight different gRNAs.

45. The library of claim 41, wherein the expression cassette encodes four to nine different gRNAs.

46. The library of claim 41, wherein the expression cassette encodes five to eight different gRNAs.

47. The library of any one of claims 36 to 40, wherein the expression cassette encodes six to eight different gRNAs.

48. The library of any one of claims 36 to 47, wherein the second allele is a naturally-occurring allele.

49. The library of any one of claims 36 to 48, wherein the second allele is not a hypomorphic allele.

50. The library of any one of claims 36 to 48, wherein the second allele is not a null allele.

51. The library of any one of claims 36 to 50, wherein the first allele contains a mutation in a regulatory region of the gene of interest.

52. The library of any one of claims 36 to 50, wherein the first allele contains a mutation in a coding sequence of the gene of interest.

53. The library of claim 51 or 52, wherein the first allele is a hypomorphic allele that results in an mRNA expression level of the gene of interest that is at least 70% lower than an allele of the gene of interest that does not contain the mutation.

54. The library of any one of claims 36 to 53, wherein each target sequence is located 50 to 500 base pairs away from at least one other target sequence.

55. The library of any one of claims 36 to 54, wherein the library contains at least 50 members.

56. The library of any one of claims 36 to 55, wherein the plant or seed is a crop plant or crop seed.

57. The library of any one of claims 36 to 56, wherein the library is a plant library and at least one member of the library contains a gRNA/endonuclease-induced mutation in the second allele.

58. The library of claim 57, wherein the gRNA/endonuclease-induced is a deletion, inversion, translocation or insertion, or a combination of structural variations thereof.

59. The library of any one of claims 36 to 58, wherein the RNA-guided endonuclease is a Cas9 or Cpf1 endonuclease.

60. A method of selecting members of a library having a phenotype of interest, the method comprising:
(a) providing a plant or seed library of any one of claims 36 to 59,
(b) selecting at least one member of the library that exhibits a phenotype of interest, and
(c) crossing the at least one member to at least one plant that does not contain the expression cassette.

61. The method of claim 60, wherein the method further comprises propagating or multiplying the plant obtained in step (c).

62. The method of claim 60 or 61, wherein the method further comprises producing a seed from the plant obtained in step (c).

63. A plant or seed obtainable, or obtained by, the method of any one of claims 60 to 62.

64. A plant or seed that is homozygous for a second allele of a gene of interest containing at least one gRNA/RNA-guided endonuclease-induced mutation obtainable, or obtained by, a process comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest,
(c) crossing the first plant to the second plant to produce a plurality of F1 hybrid plants, each F1 hybrid plant in the plurality comprising the first allele, the second allele and the expression cassette,
(d) maintaining the plurality of F1 hybrid plants under conditions that permit the gRNA/RNA-guided endonuclease to induce mutations within the target region of the second allele,
(e) selecting an F1 hybrid plant of step (d) having a phenotype of interest, and
(f) performing a cross with the F1 hybrid plant to produce a progeny plant or seed that is homozygous for the second allele containing at least one gRNA/RNA-guided endonuclease-induced mutation.

65. The plant or seed of claim 64, wherein the mutation is a deletion, inversion, translocation or insertion, or a combination of structural variations thereof.

66. A plant cell or seed cell obtainable, or obtained by, a process comprising isolating a cell from the plant or seed of claim 64 or 65.

67. An isolated DNA molecule comprising a second allele of a gene of interest containing at least one gRNA/Cas9-induced mutation or a fragment of the second allele containing the target region containing the at least one gRNA/Cas9-induced mutation, the DNA molecule obtainable, or obtained by, a process comprising isolating a DNA molecule comprising the second allele, or the fragment thereof, from the plant or seed of claim 64 or 65 or from the plant cell or seed cell of claim 66.

68. A plant library comprising a plurality of F1 hybrid plants obtainable, or obtained by, a process comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest, and
(c) crossing the first plant to the second plant to produce a plurality of F1 hybrid plants, each F1 hybrid plant in the plurality comprising the first allele, the second allele and the expression cassette.

69. A seed library comprising a plurality of F1 hybrid seeds obtainable, or obtained by, a process comprising:
(a) providing a first plant comprising
(i) a gene of interest comprising a coding sequence and having a first allele that is a hypomorphic allele or a null allele, and
(ii) an expression cassette that encodes a RNA-guided endonuclease and at least four different guide RNAs (gRNAs), each gRNA containing a sequence that is complementary to a target sequence within a target region in a second allele of the gene of interest that is different from the first allele, wherein the target region is 0 to 5000 base pairs upstream of the 5′ end of the coding sequence of the gene of interest or wherein the target region is 0 to 5000 base pairs downstream of the 3′ end of the coding sequence of the gene of interest,
(b) providing a second plant comprising the second allele of the gene of interest, and
(c) crossing the first plant to the second plant to produce a plurality of F1 hybrid seeds, each F1 hybrid seed in the plurality comprising the first allele, the second allele and the expression cassette.

70. The plant or seed library of claim 68 or 69, wherein the first plant is hemizygous for the expression cassette.

71. The plant or seed library of any one of claims 68 to 70, wherein the first plant is homozygous for the first allele and the second plant is homozygous for the second allele.

72. The plant or seed library of any one of claims 68 to 71, wherein the method further comprises maintaining the plurality of F1 hybrid plants or F1 hybrid seeds under conditions that permit the gRNA/Cas9 to induce mutations within the target region of the second allele.

73. The plant or seed library of any one of claims 68 to 72, wherein the RNA-guided endonuclease is a Cas9 or Cpf1 endonuclease.

74. A nucleic acid comprising an expression construct encoding a RNA-guided endonuclease and at least four different guide RNAs (gRNAs), each gRNA containing a sequence that is complementary to a target sequence within a target region in an allele of a gene of interest in a plant, wherein the target region is 0 to 5000 base pairs upstream of the 5′ end of the coding sequence of the gene of interest or wherein the target region is 0 to 5000 base pairs downstream of the 3′ end of the coding sequence of the gene of interest.

75. The nucleic acid of claim 74, wherein the target region comprises a regulatory region of the gene of interest.

76. The nucleic acid of claim 75, wherein the regulatory region comprises a transcription factor binding site, an RNA polymerase binding site, a TATA box, or a combination thereof.

77. The nucleic acid of claim 75 or 76, wherein the regulatory region is a promoter.

78. The nucleic acid of any one of claims 74 to 77, wherein the expression cassette encodes at least five different gRNAs.

79. The nucleic acid of claim 78, wherein the expression cassette encodes at least six different gRNAs.

80. The nucleic acid of claim 78, wherein the expression cassette encodes at least seven different gRNAs.

81. The nucleic acid of claim 78, wherein the expression cassette encodes at least eight different gRNAs.

82. The nucleic acid of any one of claims 74 to 81, wherein the expression cassette encodes four to nine different gRNAs.

83. The nucleic acid of claim 82, wherein the expression cassette encodes five to eight different gRNAs.

84. The nucleic acid of claim 82, wherein the expression cassette encodes six to eight different gRNAs.

85. The nucleic acid of any one of claims 74 to 84, wherein each target sequence is located 50 to 500 base pairs away from at least one other target sequence.

86. The nucleic acid of any one of claims 74 to 85, wherein the expression cassette contains a constitutive promoter.

87. The nucleic acid of any one of claims 74 to 86, wherein the nucleic acid is a vector.

88. The nucleic acid of any one of claims 74 to 87, wherein the plant is a crop plant.

89. The nucleic acid of any one of claims 74 to 88, wherein the nucleic acid is contained within a cell.

90. The nucleic acid of claim 89, wherein the cell is a plant cell.

91. The nucleic acid of claim 89, wherein the cell is a bacterial cell.

92. The nucleic acid of any one of claims 74 to 91, wherein the RNA-guided endonuclease is a Cas9 or Cpf1 endonuclease.

93. Use of the library of any one of claims 36 to 59 or 68 to 73, the DNA molecule of claim 67, the nucleic acid of any one of claims 74 to 92, or the F1 hybrid plant as defined in any one of the preceding claims for the production of a crop plant or seed thereof.

94. The use of claim 93, wherein the crop plant or seed thereof carries a mutation in the regulatory region of a gene that controls a commercially relevant trait.

95. The use of claim 93, wherein the crop plant or seed thereof is transgene-free.

96. A method for generating crop plants or a seed thereof with alleles that weakly affect one or more commercially relevant traits, comprising the use of the library of any one of claims 36 to 59 or 68 to 73, the DNA molecule of claim 67, the nucleic acid of any one of claims 74 to 92, or the F1 hybrid plant as defined in any one of the preceding claims.

97. The use of any one of claims 93 to 95 or method of claim 96 wherein the commercially relevant trait is a yield-related trait or a quality-related trait.

98. A crop plant or seed thereof obtainable or obtained by the use or method of any one of claims 93-97.
